SEQUENCE LISTING (34 sequences)

SEQ ID NO: 1 - DNA, 20 bp, Artificial Sequence
  Synthetic
cactctttccctacacgacg

SEQ ID NO: 2 - DNA, 20 bp, Artificial Sequence
  Synthetic
agttcagacgtgtgctcttc

SEQ ID NO: 3 - protein, 285 aa, Unknown
  Enterobacteriaceae
MKYIGAHVSAAGGLANAAIRAAEIDATAFALFTKNQRQWRAAPLTTQTIDEFKAACEKYHYTSAQILPHDSYLINLGHPVTEALEKSRDAFIDEMQRCEQLGLSLLNFHPGSHLMQISEEDCLARIAESINIALDKTQGVTAVIENTAGQGSNLGFKFEHLAAIIDGVEDKSRVGVCIDTCHAFAAGYDLRTPAECEKTFADFARIVGFKYLRGMHLNDAKSTFGSRVDRHHSLGEGNIGHDAFRWIMQDDRFDGIPLILETINPDIWAEEIAWLKAQQTEKAVA

SEQ ID NO: 4 - protein, 269 aa, Escherichia coli
MPELPEVETSRRGIEPHLVGATILHAVVRNGRLRWPVSEEIYRLSDQPVLSVQRRAKYLLLELPEGWIIIHLGMSGSLRILPEELPPEKHDHVELVMSNGKVLRYTDPRRFGAWLWTKELEGHNVLTHLGPEPLSDDFNGEYLHQKCAKKKTAIKPWLMDNKLVVGVGNIYASESLFAAGIHPDRLASSLSLAECELLARVIKAVLLRSIEQGGTTLKDFLQSDGKPGYFAQELQVYGRKGEPCRVCGTPIVATKHAQRATFYCRQCQK

SEQ ID NO: 5 - protein, 229 aa, Unknown
  Enterobacteriaceae
MANELTWHDVLAEEKQQPYFLNTLQTVASERQSGVTIYPPQKDVFNAFRFTELGDVKVVILGQDPYHGPGQAHGLAFSVRPGIAIPPSLLNMYKELENTIPGFTRPNHGYLESWARQGVLLLNTVLTVRAGQAHSHASLGWETFTDKVISLINQHREGVVFLLWGSHAQKKGAIIDKQRHHVLKAPHPSPLSAHRGFFGCNHFVLANQWLEQRGETPIDWMPVLPAESE

SEQ ID NO: 6 - protein, 289 aa, Unknown
  Trypanosoma grayi
MVQRTLLDFVRKKNTSPTCVKVDVDEESSEEVMRQSLKRTAEDMEALPPPVKKKMELDACLSSLIIDPDWRAFLHPLTSAPSFQRVCQFVEMEAASGKVILPPRELIFSAFNSTPLERVKVVLLGQDPYHNIGQAHGLCFSVRPGMRPPPSLVNMYKELSADIPGFKAPSHGYLQHWAEQGVLMLNATLTVEAHKANSHATCGWQAFTDGVIHLLSDAHKKPIVFLLWGGFARKKITLIDRKRHVVIENAHPSPLSATKWWGSRPFSKCNDALTKIGHTPIDWSLPMTV

SEQ ID NO: 7 - protein, 239 aa, Unknown
  Deinococcus maricopensis DSM 21211
MTQPDLFNPGPALPDLPPSWAAVLVDELHSPRFRALMDFVVQERAEHAVYPPEADVFNALRLTPLEDVKVLILGQDPYHGAGQAHGLAFSVQPGVRVPPSLKNIYKELQADVGFVPPKHGHLTAWARRGVLLLNAVLTVRAGEPNSHAGRGWEHVTDAVIRALNAREERVVFVLWGAYARKKAKLITAPQHVIIESAHPSPLSVAKFLGTKPFSQVNAALDEAGRGAIDWQLPADPNEQ

SEQ ID NO: 8 - protein, 263 aa, Escherichia coli
MPEGPEIRRAADNLEAAIKGKPLTDVWFAFPQLKPYQSQLIGQHVTHVETRGKALLTHFSNDLTLYSHNQLYGVWRVVDTGEEPQTTRVLRVKLQTADKTILLYSASDIEMLTPEQLTTHPFLQRVGPDVLDPNLTPEVVKERLLSPRFRNRQFAGLLLDQAFLAGLGNYLRVEILWQVGLTGNHKAKDLNAAQLDALAHALLEIPRFSYATRGQVDENKHHGALFRFKVFHRDGEPCERCGSIIEKTTLSSRPFYWCPGCQH

SEQ ID NO: 9 - protein, 263 aa, Unknown
  Klebsiella
MPEGPEIRRAADKLEAAIKGEPLTNVWFAFPQLQPYQTQLIGQRVTHIATRGKALLTHFSGGLTLYSHNQLYGVWRVVDAGVEPQSNRVLRVRLQTASKAILLYSASDIDILTAEQVANHPFLLRVGPDVLDMTLTAEQVKARLLSAKFRNRQFSGLLLDQAFLAGLGNYLRVEILWQVGLSGKRKAAELSDSQLDALAHALLDIPRLSYRTRGLVDDNKHHGALFRFKVFHRDGERCERCGGIIEKTTLSSRPFYWCPGCQH

SEQ ID NO: 10 - protein, 370 aa, Escherichia coli
MIWQLTDDKRWSALRQRFSWVEEMHHTPQDPEHHGEGDVGVHTEMVLNALITLPEFQQLPAQQQEVLWAAALLHDVEKRSTTVQENGRIQSPGHARRGELTARQILWRDIPTPFVLREQIVALVRLHGLPLWLLERPEPERLLLTAAMRIDTRLLALLARADLLGRQSPDQQSMLERIDLFELFCHEQQCWGKMRPFVSDSARWHYLTHEQSSPDFVPWEAEPFEVILLCGLPGMGKDRYISEQCQGMDVISLDDMRRRINASPDDKTATGRIVQQAKEEARVFLRQKKPFIWNATNITRQLRSQLISLFTAYGARVKIVYLEVPWAQWNQQNARREYAVPEAVVMRMASRLEVPQPDEAHSVEYRMTDR

SEQ ID NO: 11 - protein, 440 aa, Escherichia coli
MKKLIALKHKLDEMKAMGTNAKKEALANMDDFEQSMVSLMLNPFIRFGVKKYKVAEPLSESVPSDEKAIDILNKLASRELTGNAAIAAVESIVASMCADGQDVFRRFLLKDPKAGVGISLCNKVFENPIPKFEVQLASPYKEKGDKYPFKPNPKAKWPMIGSLKLDGLRVICEVIVDEEEVNFLSRTGNPITSLDHLKPAMLELGKLSGHKHIFFDGEGTAGSFNQSVSALRKKNVQAIGAIYHVFDFFLPEWRAQAKSKEYAKTGMKLKERLAMLVALFKNDRSEGYAQDIHLHPFYIIHSHEDFIERFMKRLDDNQEGEMGKDPNSVYEFKRTRSWWKLKDEDSEDGEIIDFEPGDPDSGFANTLGKIVIRLENGVIVRASGIKHKYLDEIWNNKEKYRGRIVEVHCHEKTPDGSLRHPRLKWPRCLRDTEDRIGDKE

SEQ ID NO: 12 - protein, 282 aa, Unknown
  Campylobacter
MRFVFLICCACLVFANEILLLSKFDKQDFNSKDFNAYLMSEKLDGVRGIWDGKYLKTRQNYKIKTPDFFTKNFPPFAIDGELWIARNKFDEISALIRSGDSNLTLWKEVTYNIFDVPNACEEFQISTCTLKNRLAVLEEYLQKYPSAYIKIISQIPVENQNNLNQFYESIIKNQGEGIVIRKNLSPYEKGRSKNATKLKPYDDAECELVGFRKGKGKFENQVGALLCKMPNGQIIKIGSGLKDEDRKNPPKIGSIVTYKFNGLTKNSLPRFPVFLRIRDENP

SEQ ID NO: 13 - protein, 303 aa, Unknown
  Pseudomonas phage EPa61
MKPMFASMADPGLLEGQLPIFMSPKIDGFRAFIHDGHALTRSFKPQANAAINQYLSNPIFNGLDGELVCGDITDPKVFQKSSGDLRRRDGEPDWSFHVFDDFTDPRMPTDERLDAAEHRVKELRERFEMTRIHLVEQELVTSVEQMTETELRHVGLGFEGSIGKKKNGIYKFGRSTAKEGHCVKFKRYDSEEAEIIGVEELMINENEAFIDELGYTARSSHAENLVPSGMVGSFVCRNEKVWPGQTFRVSASSITHDEKQRLWKDRESLNGQVIRFTHFPHGAKDKPRHAVFDCWLDGWGASH

SEQ ID NO: 14 - DNA, 45 bp, Artificial Sequence
  Synthetic
gcgtcaccagccacgcgagccggatgaggatccgtgacgcgaagt

SEQ ID NO: 15 - DNA, 70 bp, Artificial Sequence
  Synthetic
gcgtcaccagccacgcgagccggatgaggatccgtgacgcgaagtcctggtaccgccgctcgcttccgac

SEQ ID NO: 16 - DNA, 80 bp, Artificial Sequence
  Synthetic
gcgtcaccagccacgcgagccggatgaggatccgtgacgcgaagtcctggtaccgccgctcgcttccgaccggttctcca

SEQ ID NO: 17 - DNA, 90 bp, Artificial Sequence
  Synthetic
gcgtcaccagccacgcgagccggatgaggatccgtgacgcgaagtcctggtaccgccgctcgcttccgaccggttctccaccgagcgacc

SEQ ID NO: 18 - DNA, 100 bp, Artificial Sequence
  Synthetic
gcgtcaccagccacgcgagccggatgaggatccgtgacgcgaagtcctggtaccgccgctcgcttccgaccggttctccaccgagcgacctaatattaat

SEQ ID NO: 19 - DNA, 70 bp, Artificial Sequence
  Synthetic
gtcggaagcgagcggcggtaccaggacttcgcgtcacggatcctcatccggctcgcgtggctggtgacgc

SEQ ID NO: 20 - DNA, 80 bp, Artificial Sequence
  Synthetic
tggagaaccggtcggaagcgagcggcggtaccaggacttcgcgtcacggatcctcatccggctcgcgtggctggtgacgc

SEQ ID NO: 21 - DNA, 90 bp, Artificial Sequence
  Synthetic
ggtcgctcggtggagaaccggtcggaagcgagcggcggtaccaggacttcgcgtcacggatcctcatccggctcgcgtggctggtgacgc

SEQ ID NO: 22 - DNA, 100 bp, Artificial Sequence
  Synthetic
  misc_feature (96)..(100): linked via phosphorothioate bonds
attaatattaggtcgctcggtggagaaccggtcggaagcgagcggcggtaccaggacttcgcgtcacggatcctcatccggctcgcgtggctggtgacgc

SEQ ID NO: 23 - DNA, 100 bp, Artificial Sequence; identical to SEQ ID NO: 22
  Synthetic
  modified_base (32)..(32): i8oxod guanine
  misc_feature (97)..(100): linked via phosphorothioate bonds

SEQ ID NO: 24 - DNA, 100 bp, Artificial Sequence
  Synthetic
  misc_feature (97)..(100): linked via phosphorothioate bonds
attaatattaggtcgctcggtggagaaccugtcggaagcgagcggcggtaccaggacttcgcgtcacggatcctcatccggctcgcgtggctggtgacgc

SEQ ID NO: 25 - DNA, 30 bp, Artificial Sequence
  Synthetic
cggttctccaccgagcgacctaatattaat

SEQ ID NO: 26 - DNA, 29 bp, Artificial Sequence
  Synthetic
ggttctccaccgagcgacctaatattaat

SEQ ID NO: 27 - DNA, 25 bp, Artificial Sequence
  Synthetic
ctccaccgagcgacctaatattaat

SEQ ID NO: 28 - DNA, 166 bp, Artificial Sequence
  Synthetic
gtcaagggtaatggacagtaggtgtggtggaacatacttccagcactcaagaagctgaagcaggcagatctctgtcagttcatgaccactgctgtctacatggtgagctccaagccagccaggcaagaagtgacactcaggtctcgcattgctcagacggcaggca

SEQ ID NO: 29 - DNA, 167 bp, Artificial Sequence
  Synthetic
tgcctgccgtctgagcaatgcgagacctgagtgtcacttcttgcctggctggcttggagctcaccatgtagacagcagtggtcatgaactgacagagatctgcctgcttcagcttcttgagtgctggaagtatgttccaccacacctactgtccattacccttgaca

SEQ ID NO: 30 - DNA, 156 bp, Artificial Sequence
  Synthetic
gtcaagggtaatggacagtaggtgtggtggaacatacttccagcactcaagaagctgaagcaggcagatctctgtcagttcatgaccactgctgtctacatggtgagctccaagccagccaggcaagaagtgacactcaggtctcgcattgctcag

SEQ ID NO: 31 - DNA, 86 bp, Artificial Sequence
  Synthetic
gtcaagggtaatggacagtaggtgtggtggaacatacttccagcactcaagaagctgaagcaggcagatctctgtcagttcatgac

SEQ ID NO: 32 - DNA, 80 bp, Artificial Sequence
  Synthetic
cactgctgtctacatggtgagctccaagccagccaggcaagaagtgacactcaggtctcgcattgctcagacggcaggca

SEQ ID NO: 33 - DNA, 79 bp, Artificial Sequence
  Synthetic
actgctgtctacatggtgagctccaagccagccaggcaagaagtgacactcaggtctcgcattgctcagacggcaggca

SEQ ID NO: 34 - DNA, 101 bp, Artificial Sequence
  Synthetic
atgagaacaggtgacgccgacccgaccagacgctttgtgtcgaagcgcgaggatctggagcgaactgctgcgcctcgatgagccgctcgcttccgtccgtt

(19) United States
(12) Patent Application Publication
(10) Pub. No.: US 2024/0110223 A1
(43) Pub. Date: Apr. 4, 2024

(54) METHODS FOR DUPLEX REPAIR

(71) Applicant: The Broad Institute, Inc., Cambridge, MA (US)

(72) Inventors: Viktor A. Adalsteinsson, Cambridge, MA (US); Kan Xiong, Cambridge, MA (US); Douglas Shea, Cambridge, MA (US); Justin Rhoades, Cambridge, MA (US)

(73) Assignee: The Broad Institute, Inc., Cambridge, MA (US)

(21) Appl. No.: 18/266,555

(22) PCT Filed: Dec. 10, 2021
(86) PCT No.: PCT/US2021/062936

§ 371 Date: Jun. 9, 2023

Related U.S. Application Data
(60) Provisional application No. 63/217,007, filed on Jun. 30, 2021.
(60) Provisional application No. 63/191,914, filed on May 21, 2021.
(60) Provisional application No. 63/191,320, filed on May 20, 2021.
(60) Provisional application No. 63/143,397, filed on Jan. 29, 2021.
(60) Provisional application No. 63/124,700, filed on Dec. 11, 2020.

(51) Int. Cl. C12Q 1/6806

(52) U.S. Cl. CPC C12Q 1/6806

(57) ABSTRACT

Methods and kits are disclosed related to preparing a nucleic acid sample for sequencing that minimizes propagation of false mutations due to amplification of nucleotide damage or alterations confined to one strand wherein at least a portion of the sample is double-stranded.

### CROSS REFERENCE TO RELATED APPLICATIONS

This application claims the benefit under 35 U.S.C. § 119(e) of U.S. Provisional Application No. 63/124,700, filed Dec. 11, 2020, entitled “METHODS FOR DUPLEX REPAIR,” U.S. Provisional Application No. 63/143,397, filed Jan. 29, 2021, entitled “METHODS FOR DUPLEX REPAIR,” U.S. Provisional Application No. 63/191,320, filed May 20, 2021, entitled “METHODS FOR DUPLEX REPAIR,” U.S. Provisional Application No. 63/191,914, filed May 21, 2021, entitled “METHODS FOR DUPLEX REPAIR,” and U.S. Provisional Application No. 63/217,007, filed Jun. 30, 2021, entitled “METHODS FOR DUPLEX REPAIR,” the entire disclosures of each of which are hereby incorporated by reference in their entireties.

### SEQUENCE LISTING

The instant applications contains a Sequence Listing which has been submitted in ASCII format via EFS-Web and is hereby incorporated by reference in its entirety. Said ASCII copy, created on Dec. 10, 2021, is named B119570118WO00-SEQ-GJM.txt and is 35,088 bytes in size.

### BACKGROUND

Accurate sequencing of nucleic acids is crucial in many areas (e.g., biomedical research and development, clinical diagnostics and therapy) but challenging. While the cost of DNA sequencing has declined one-million-fold since the early 2000's, next generation sequencing (NGS) error rates have remained high (˜0.1%), a number which has remained relatively unchanged. This error rate makes it difficult to resolve true mutations, particularly those which are present at low abundance. Higher fidelity can be attained by reading each sequence multiple times; for example, by requiring a consensus of reads from both strands of each original DNA duplex, techniques such as “duplex sequencing” can achieve error rates as low as 0.0001-0.00001% (1×10⁻⁶-1×10⁻⁷). Yet, their accuracy may fail in areas which are paramount to their use for discerning true mutations. For instance, error rates for heavily-damaged (e.g., oxidized, deaminated, as further described hereinbelow) samples such as formalin-fixed tumor biopsies could be >100-fold higher. This is because existing methods which are needed to prepare nucleic acids for sequencing could resynthesize portions of each DNA duplex and render amplifiable lesions or alterations originally confined to one strand indiscernible from true mutations on both strands. Accordingly, new methods are needed to improve the accuracy of existing methods, such as duplex sequencing, which require a consensus of sequences from both strands of each duplex, without compromising mutation detection.

### SUMMARY OF THE INVENTION

Existing methods used for nucleic acid preparation perform a number of activities and steps. The existing methods, known as “end repair” (ER) and “dA-tailing” (AT) (ER/AT), are used to blunt and phosphorylate DNA fragments, and perform non-templated addition of deoxyadenosine monophosphate (“dAMP”) to the 3′ ends, respectively, in preparation for ligation of dTMP-tailed sequencing adapters (FIG. 1). ER and AT are performed either sequentially or within a “one-pot” reaction (e.g., the entirety of the process and method occur concurrently within one reaction vessel without separation of steps), and employ DNA polymerase(s) which are intended to digest 3′ overhangs and fill-in 5′ overhangs, and to leave a single dAMP on each 3′ end of the strands of the duplex. Yet, ER/AT (either on its own, or in combination with pretreatments, such as NEB PreCR® or ExoVII—e.g., see FIG. 34 and FIGS. 35A-35C) traditionally involve the use of one or more DNA polymerase(s) which bear 5′ exonuclease and/or strand displacement activity. It was thus hypothesized that extensive strand resynthesis could occur from internal nicks and gaps within the duplex, and from long 5′ overhangs. If resynthesis occurs in the presence of an amplifiable lesion or alteration originally confined to one strand, it may, or is likely to, copy errors to both strands and render them indistinguishable from true mutations on both strands. While this source of false discovery in duplex sequencing is most clearly seen at fragment ends where short 5′ overhangs are often filled in (FIG. 2C), it is shown herein that such errors could also span much deeper into fragments given (i) the 5′ exonuclease and strand-displacement activities of polymerases such as Taq and Klenow which are commonly used in ER/AT and (ii) the varied extents of backbone damages, induced by multiple intrinsic or extrinsic factors, that serve as ‘priming sites’ for strand resynthesis (e.g., nicks, gaps). This could explain why a long tail of errors was observed that decreased with distance from 3′ fragment end in the heavily damaged FFPE tumor DNA samples, which had ˜100-fold higher error rates than 271 cell-free DNA (cfDNA) samples (FIG. 2C). This mechanism has also been confirmed through experiments involving treatment of synthetic oligonucleotides bearing nicks, gaps, and overhangs with traditional ER/AT kits (FIG. 2B and FIG. 3A). While errors at fragment ends can be mitigated through in silico trimming of fragment ends, errors which arise within the interior of each fragment (or, beyond a prespecified distance from fragment end, e.g., >12 bp) cannot be resolved in this manner without severely compromising the yield of DNA sequencing data. This means that while duplex sequencing can, in theory, discern base damage errors on one strand, its ability, in practice, depends on the quality of the starting material, which for a multitude of reasons, is deeply problematic. For example, prior to ER/AT, samples are fragmented to prepare a library. This fragmentation breaks apart a nucleic acid into small fragments. This can be accomplished, physically (e.g., by sonication or physical force), enzymatically, or chemically. However, all forms of fragmentation inherently damage the strands to break them and can induce off-target damage (e.g., overhangs, nicks, gaps, damaged bases).

Disclosed herein is a new ER/AT method called Duplex-Repair (DR), which minimizes and/or eliminates many of the problems inherent to existing methods. For example, without limitation, DR minimizes strand resynthesis prior to ligation of NGS adapters, which significantly limits false mutation discovery. As shown herein, by minimizing this resynthesis, DR addresses a major Achilles' heel of duplex sequencing, and other related methods, which rely upon a consensus of sequences from both strands of each duplex, to provide maximum accuracy and robustness.

Accordingly, in some aspects, the disclosure relates to a method of preparing a nucleic acid sample (sample) for sequencing that minimizes propagation of false mutations due to amplification of nucleotide damage or alterations originally confined to one strand, wherein at least a portion of the sample is double-stranded, comprising adding a sample to a reaction vessel and: (a) contacting the sample to one or more enzymes capable of: (i) excising one or more damaged bases from the sample; (ii) cleaving one or more abasic sites, and processing the resulting ends to be compatible with extension by a DNA polymerase and/or ligation by a DNA ligase; and (iii) digesting 5′ overhangs; (b) contacting the sample with one or more of: (i) a DNA-dependent DNA polymerase lacking both strand displacement and 5′ exonuclease activities but capable of filling in single-stranded segments of the sample and digesting 3′ overhangs of the sample; and (ii) an enzyme capable of phosphorylating the 5′ ends of the strands of the sample; (c) contacting the sample with a DNA ligase capable of sealing nicks; and (d) preparing the sample for adapter ligation, wherein the preparing comprises adding dAMP to the 3′ ends of the strands of the sample (dA-tailing). Such enzymes are well-known in the art and can be obtained from any suitable source, including commercial sources, such as New England BioLabs, AMSBIO, and Sigma-Aldrich. A person having ordinary skill in the art will understand based on the name of the enzymes disclosed herein the identity of the enzymes disclosed herein and how to obtain said enzymes without undue experimentation.

In some embodiments, dA-tailing comprises contacting a sample with an enzyme capable of incorporating one deoxyanenosine monophosphate (dAMP) to each 3′ end of the strands of the sample and contacting the sample with dNTPs. In some embodiments, enzymes and/or dNTPs used in steps (a)-(c) of the methods of the disclosure are substantially removed from the reaction vessel prior to dA-tailing. In some embodiments, dNTPs substantially comprise dATPs.

In some embodiments, a sample is contacted by the one or more enzymes of step (a) and incubated for at least 5 minutes (min) prior to proceeding with any subsequent steps of the method. In some embodiments, a sample is contacted by the one or more enzymes of step (a) and incubated for at least 25 minutes (min) prior to proceeding with any subsequent steps of the method. In some embodiments a sample is contacted by the one or more enzymes of step (a) and incubated for at least 30 minutes (min) prior to proceeding with any subsequent steps of the method. In some embodiments, a sample is contacted by the one or more enzymes of step (b) and incubated for at least 5 minutes (min) prior to proceeding with any subsequent steps of the method. In some embodiments, a sample is contacted by the one or more enzymes of step (b) and incubated for at least 25 minutes (min) prior to proceeding with any subsequent steps of the method. In some embodiments, a sample is contacted by the one or more enzymes of step (b) and incubated for at least 30 minutes (min) prior to proceeding with any subsequent steps of the method. In some embodiments, a sample is contacted by the one or more enzymes of step (c) and incubated for at least 15 minutes (min) prior to proceeding with any subsequent steps of the method. In some embodiments, a sample is contacted by the one or more enzymes of step (c) and incubated for at least 30 minutes (min) prior to proceeding with any subsequent steps of the method. In some embodiments, a sample is contacted by the one or more enzymes of step (c) and incubated for at least 45 minutes (min) prior to proceeding with any subsequent steps of the method. In some embodiments, a sample is contacted by the one or more enzymes of step (d) and incubated for at least 40 minutes (min) prior to proceeding with any subsequent steps of the method. In some embodiments, a sample is contacted by the one or more enzymes of step (d) and incubated for at least 60 minutes (min) prior to proceeding with any subsequent steps of the method. In some embodiments, a sample is contacted by the one or more enzymes of step (d) and incubated for at least 70 minutes (min) prior to proceeding with any subsequent steps of the method.

In some embodiments, step (a) is carried out at a temperature between about 32 degrees Celsius (° C.) to about 42° C. In some embodiments, step (a) is carried out at a temperature between about 35° C. to about 39° C. In some embodiments, step (b) is carried out at a temperature between about 32° C. to about 42° C. In some embodiments, step (b) is carried out at a temperature between about 35° C. to about 39° C. In some embodiments, step (c) is carried out at a temperature between about 30° C. to about 70° C. In some embodiments, step (c) is carried out at a temperature between about 33° C. to about 67° C. In some embodiments, step (d) is carried out at a temperature between about 18° C. to about 69° C. In some embodiments, step (d) is carried out at a temperature between about 20° C. to about 67° C.

In some embodiments, prior to step (a) a sample has been: (i) fragmented; or (ii) cleaved and tagged (tagmented). In some embodiments, fragmentation is by: (a) physical fragmentation; (b) enzymatic fragmentation; and/or (c) chemical fragmentation. In some embodiments, fragmentation is by physical fragmentation. In some embodiments, fragmentation is by enzymatic fragmentation. In some embodiments, fragmentation is by chemical fragmentation.

In some embodiments, step (a) comprises contacting the sample with one or more enzymes selected from the group consisting of: (1) endonuclease IV (EndoIV); (2) formamidopyrimidine [fapy]-DNA glycosylase (Fpg); (3) uracil-DNA glycosylase (UDG); (4) T4 pyrimidine DNA glycosylase (T4 PDG); (5) endonuclease VIII (EndoVIII) and (6) exonuclease VII (ExoVII). Such enzymes are well-known in the art and can be obtained from any suitable source, including commercial sources, such as New England BioLabs, AMSBIO, and Sigma-Aldrich. A person having ordinary skill in the art will understand based on the name of the enzymes disclosed herein the identity of the enzymes disclosed herein and how to obtain said enzymes without undue experimentation.

In some embodiments, the activity of the one or more enzymes catalyze the following DNA modifications on the sample: (1) excision of damaged bases; and (2) cleaving one or more abasic sites, and processing the resulting ends to be compatible with extension by a DNA polymerase and/or ligation by a DNA ligase. In some embodiments, activity of the one or more enzymes is sequential or simultaneous.

In some embodiments, a damaged base is selected from the group consisting of: uracil; 8′oxoG; an oxidized pyrimidine; and a cyclobutane pyrimidine dimer.

In some embodiments, a 5′ overhang of at least one strand of the sample is at least 10 nucleobases in length. In some embodiments, a 5′ overhang of at least one strand of the sample is at least 75 nucleobases in length. In some embodiments, a 3′ overhang of at least one strand of the sample is at least 10 nucleobases in length. In some embodiments, a 3′ overhang of at least one strand of the sample is at least 75 nucleobases in length.

In some embodiments, one or more enzymes digests a 5′ overhang of at least one strand of the sample to less than 16 nucleobases in length. In some embodiments, one or more enzymes digests a 5′ overhang of at least one strand of the sample to less than 8 nucleobases in length. In some embodiments, one or more enzymes digests a 3′ overhang of at least one strand of the sample to less than 16 nucleobases in length. In some embodiments, one or more enzymes digests a 3′ overhang of at least one strand of the sample to less than 8 nucleobases in length.

In some embodiments, endonuclease IV (EndoIV) cleaves abasic sites. In some embodiments, formamidopyrimidine [fapy]-DNA glycosylase excises damaged purines. In some embodiments, uracil-DNA glycosylase (UDG) excises uracil. In some embodiments, T4 pyrimidine DNA glycosylase (T4 PDG) excises cyclobutane pyrimidine dimers. In some embodiments, endonuclease VIII (EndoVIII) excises damaged pyrimidines. In some embodiments, DNA ligase is a HiFi Taq DNA ligase. Such enzymes are well-known in the art and can be obtained from any suitable source, including commercial sources, such as New England BioLabs, AMSBIO, and Sigma-Aldrich. A person having ordinary skill in the art will understand based on the name of the enzymes disclosed herein the identity of the enzymes disclosed herein and how to obtain said enzymes without undue experimentation.

In some embodiments, step (b) of the methods of the disclosure comprises contacting the DNA fragment with a polynucleotide kinase (Pnk). In some embodiments, a Pnk is a T4 polynucleotide kinase. In some embodiments, the DNA polymerase used in step (b) of the methods of the disclosure is T4 DNA polymerase. In some embodiments, the DNA polymerase(s) used in step (d) of the methods of the disclosure comprise Taq polymerase and/or Klenow fragment. Such enzymes are well-known in the art and can be obtained from any suitable source, including commercial sources, such as New England BioLabs, AMSBIO, and Sigma-Aldrich. A person having ordinary skill in the art will understand based on the name of the enzymes disclosed herein the identity of the enzymes disclosed herein and how to obtain said enzymes without undue experimentation.

In some embodiments of any of the methods of the disclosure: (a) an endonuclease IV (EndoIV) comprises an amino acid sequence with at least 70% identity to SEQ ID NO: 3 or any known endonuclease IV sequence; (b) a formamidopyrimidine [fapy]-DNA glycosylase (Fpg) comprises an amino acid sequence with at least 70% identity to SEQ ID NO: 4 or any known formamidopyrimidine [fapy]-DNA glycosylase sequence; (c) an uracil-DNA glycosylase (UDG) comprises an amino acid sequence with at least 70% identity to an amino acid sequence selected from the group consisting of: SEQ ID NO: 5-7 or any known uracil-DNA glycosylase (UDG) sequence; (d) a T4 pyrimidine DNA glycosylase (T4 PDG) comprises an amino acid sequence with at least 70% identity to an amino acid sequence selected from any known T4 pyrimidine DNA glycosylase sequence; and/or (e) an endonuclease VIII (EndoVIII) comprises an amino acid sequence with at least 70% identity to an amino acid sequence selected from the group consisting of: SEQ ID NO: 8-9 or any known endonuclease VIII sequence.

In some embodiments of any of the methods of the disclosure, a polynucleotide kinase comprises an amino acid sequence with at least 70% identity to SEQ ID NO: 10.

In some embodiments of any of the methods of the disclosure: (1) a DNA-dependent DNA polymerase comprises an amino acid sequence with at least 70% identity to any known DNA-dependent DNA polymerase sequence; and/or (2) a DNA ligase comprises an amino acid sequence with at least 70% identity to an amino acid sequence selected from the group consisting of: SEQ ID NO: 11-13 or any known DNA ligase sequence.

In some aspects, the disclosure relates to a method of duplex sequencing that mitigates false mutation detection, comprising: (A1) obtaining a nucleic acid to be sequenced; (A2) performing the method of embodiment 1 or any one of embodiments 2-51; (A3) duplex sequencing the sample; and (A4) identifying mutations by computational analysis.

In some aspects, the computational analysis requires trimming the ends of fragments (e.g., last 12 bp) to avoid false mutation detection in the limited regions at fragment ends where some resynthesis still occurs.

In some aspects, the disclosure relates to a method of reducing artifact in duplex sequencing, comprising: (A1) obtaining a nucleic acid to be sequenced; (A2) performing the method of embodiment 1 or any one of embodiments 2-51; and (A3) duplex sequencing the sample.

In some aspects, the disclosure relates to a method of reducing synthetic strand synthesis during nucleic acid sample preparation for sequencing, comprising: (A1) obtaining a nucleic acid to be sequenced; and (A2) performing the method of embodiment 1 or any one of embodiments 2-51.

In some aspects, the disclosure relates to a method of increasing the accuracy of mutation identification, comprising: (A1) obtaining a nucleic acid to be sequenced; (A2) performing the method of embodiment 1 or any one of embodiments 2-51; (A3) duplex sequencing the sample; and (A4) identifying mutations by computational analysis.

In some aspects, the disclosure relates to a kit comprising: (a) reagents to perform any of the methods of the disclosure; and (b) a container. In some embodiments, a kit further comprises a reaction vessel. In some embodiments, reagents of the kit comprise: (a) one or more of: endonuclease IV (EndoIV); exonuclease VII (Exo VII), formamidopyrimidine [fapy]-DNA glycosylase (Fpg); uracil-DNA glycosylase (UDG); T4 DNA polymerase; T4 pyrimidine DNA glycosylase (T4 PDG); T4 polynucleotide kinase (T4 Pnk); Klenow fragment; HiFi Taq ligase; Taq polymerase; and/or endonuclease VIII (EndoVIII); and/or (b) dNTPs. In some embodiments, a kit further comprises reagents and materials to fragment the sample.

In some aspects, the disclosure relates to a method of preparing a nucleic acid sample (sample) wherein at least a portion of the sample is double-stranded, comprising adding a sample to a reaction vessel and: (a) contacting the sample with one or more enzymes capable of: (i) phosphorylating the 5′ ends of the strands of the sample; adding a 3′ hydroxyl moiety to the 3′ ends of the strands of the sample; and (ii) sealing nicks; (b) contacting the sample with one or more of an enzyme capable of removing the 5′ and 3′ overhangs while also digesting gap regions to produce blunted duplexes; and (c) adding deoxyadenosine monophosphate (dAMP) to the 3′ ends of the strands of the sample (dA-tailing).

In some embodiments, a method of the present disclosure comprises use of an enzyme wherein the enzyme comprises: T4 polynucleotide kinase, HiFi Taq Ligase, or a combination thereof. In some embodiments, a method of the present disclosure comprises use of an enzyme wherein the enzyme comprises Nuclease S1.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. For purposes of clarity, not every component may be labeled in every drawing. It is to be understood that the data illustrated in the drawings in no way limit the scope of the disclosure. In the drawings:

FIG. 1 shows a comparison of a conventional method of duplex preparation (End-Repair and dA-tailing (ER/AT) and the duplex repair method of the instant disclosure (“Duplex-Repair”). Non-limiting advantages provided by Duplex-Repair include that Duplex-Repair limits polymerization prior to adapter ligation to ensure that most duplex bases sequenced were natively present in the original input DNA, and that base damage errors or other mismatches originally confined to one strand are not copied to both strands, as could happen with commercial ER/AT methods.

FIGS. 2A-2D show a method of quantifying strand resynthesis using ER/AT and quantification of strand resynthesis during ER/AT using a KAPA® HyperPrep kit. FIG. 2A is a schematic of a method for quantifying fill-in bases during ER/AT. FIG. 2B shows measured interpulse duration (IPD; in frames) as a function of the base position on five synthetic oligonucleotides. Longer IPDs, gray if greater than 60 frames, result from modified bases. Vertical dashed lines indicate where fill-in is expected to start during ER/AT. FIG. 2C shows measured IPD as a function of the base position on a healthy donor cfDNA sample. FIG. 2D shows graphs of the number of base errors measured against the distance the base is from the fragment end. Aggregate duplex error rates for 271 cfDNA samples v. 2 formalin-fixed paraffin-embedded (FFPE) tumor biopsies (left panel, top and bottom). Measured interpulse duration (IPD; in frames) as a function of the base position on four highlighted duplexes that underwent extensive strand resynthesis (right panel).

FIGS. 3A-3C shows the performance of Duplex-Repair. FIG. 3A shows the performance of the Duplex-Repair approach, in comparison to conventional ER/AT, on multiple different synthetic oligonucleotides as determined by capillary electrophoresis (i-vii). FIG. 3B shows measured duplex sequencing error rates using Duplex-Repair v. commercial ER/AT and the IDT xGEN ‘pan-cancer’ panel on healthy donor cfDNA treated with varied amounts of DNase I (to induce nicks) and CuCl₂/H₂O₂ (to induce oxidative damage). FIG. 3C shows duplex sequencing error rates after using Duplex-Repair v. conventional ER/AT to repair formalin fixed tumor DNA. The wider error bars for Duplex-Repair samples were due to fewer total duplexes sequenced.

FIG. 4 shows measured duplex sequencing error rates for different mutations using commercial ER/AT and the IDT xGEN ‘pan-cancer’ panel on healthy donor cfDNA treated with varied amounts of DNase I and CuCl₂/H₂O₂. The observed increased error rate of Cytosine to Adenine (C->A) mutation with increasing concentrations of DNase 1 and CuCl₂/H₂O₂ is consistent with the mutation signature of CuCl₂/H₂O₂ (Lee et al., Nucleic Acids Res., 2002).

FIG. 5 is a schematic showing the workflow of Duplex-Repair.

FIG. 6 shows capillary electrophoresis results demonstrating that T4 DNA polymerase efficiently fills in a 23-nucleotide gap on a dsDNA.

FIGS. 7A-7B show the characterization of Duplex-Repair using capillary electrophoresis. FIG. 7A shows an overview of Duplex-Repair vs. conventional ER/AT methods. FIG. 7B is a schematic of the major products of various synthetic duplexes subjected to each step of Duplex-Repair and conventional ER/AT as determined by capillary electrophoresis (Raw traces are in FIG. 14). The non-fluorophore-tagged ends of the synthetic molecules are depicted, and fragment sizes are drawn to scale. Duplexes demarcated by asterisks (*) do not contain fluorophores and were not directly observed by capillary electrophoresis; however, their presence is predicted due to the characterized activities of UDG and FPG. Regions of strand resynthesis are illustrated as dashed lines.

FIG. 8 shows schematics of oligos used for capillary electrophoresis and quantifying strand resynthesis with PacBio sequencing.

FIGS. 9A-9B show linear regression of measured capillary electrophoresis peak locations vs. true lengths for (FIG. 9A) 6-FAM-tagged and (FIG. 9B) ATTO-550 tagged oligonucleotides. True lengths of oligonucleotides were confirmed by IDT's mass spectrometry analysis (data not shown).

FIGS. 10A-10B show the measured library conversion efficiencies of Duplex-Repair vs. the Kapa Hyper kit as a function of a gDNA input by using a ddPCR assay. The library conversion efficiencies of Duplex-Repair are comparable to library conversion efficiencies with conventional ER/AT using the Kapa Hyper kit. ddPCR primers used are detailed in Example 2.

FIG. 11 shows the establishment of an assay for quantifying the number of bases resynthesized during ER/AT. Histogram of aggregate bases and their IPDs, labeled as original or fill-in based on which region of the synthetic oligos they were derived from. Regions that divide original and fill-in regions were avoided for collection.

FIG. 12 shows measured interpulse duration (IPD; in frames) (i) and predicted percentage of bases resynthesized (ii) as a function of the base position on five synthetic oligonucleotides treated with conventional ER/AT and with modified dNTPs. Longer IPDs, colored light gray if greater than 60 frames, result from modified bases. Dashed lines indicate where resynthesis is expected to start during ER/AT.

FIGS. 13A-13C show the quantification of strand resynthesis using single-molecule real-time sequencing. FIG. 13A shows measured interpulse duration (IPD; in frames) (i) and predicted percentage of bases resynthesized (ii) as a function of the base position on five synthetic oligonucleotides treated with conventional ER/AT using methylated dNTPs. Longer IPDs, colored light gray if greater than 60 frames, result from methylated bases. Dashed lines indicate where fill-in is expected to start during ER/AT. FIG. 13B shows measured average IPD as a function of the distance of the interrogated base from either 3′ end of each duplex for five healthy donor cfDNA samples treated with conventional ER/AT and with standard or modified dNTPs; the insert shows fraction of bases resynthesized>12 bases from either end of each duplex for cfDNA samples and FFPE tumor biopsies. FIG. 13C shows the fraction of duplex DNA strands with ≥X bases resynthesized as a function of the number of bases resynthesized, X, for one damaged cfDNA (HD 78 cfDNA treated with 100 uM CuCl₂/H₂O₂ and 2 mU DNase I) and one FFPE tumor biopsy treated with conventional ER/AT or Duplex-Repair.

FIG. 14 shows capillary electrophoresis analysis of synthetic duplexes subjected to each step of Duplex-Repair, versus conventional ER/AT. Each step of duplex repair imparts its intended functionality in producing the intended major product as depicted in FIGS. 7A-7B to minimize strand resynthesis seen with Conventional ER/AT. Oligonucleotides with a (i) 5′ overhang, (ii) 3′overhang, (iii) nick, (iv) 1 nucleotide gap, (v) 5 nucleotide gap, (vi) uracil across from a 1 nucleotide gap, and (vii) 8oxoG across from a 1 nucleotide gap were subjected to conventional ER/AT and each step of Duplex Repair and sent for capillary electrophoresis. The top strand of each oligonucleotide was labelled with 6-FAM on the 5′ end and the bottom strand of each oligonucleotide was labelled with ATTO-550 on the 3′ end.

FIG. 15 shows the characterization of the activity of key enzymes in the lesion repair enzyme cocktail by capillary electrophoresis. The activity of key enzymes to rectify each damage motif (middle) is not impacted by other enzymes in the lesion repair enzyme cocktail (bottom). The “lesion repair” condition indicates treatment with Endonuclease IV (EndoIV), Formamidopyrimidine [fapy]-DNA glycosylase (Fpg), Uracil-DNA glycosylase (UDG), T4 pyrimidine DNA glycosylase (T4 PDG), and Endonuclease VIII (EndoVIII), and Exonuclease VII (ExoVII).

FIG. 16 shows the characterization of the activity of T4 DNA polymerase and T4 polynucleotide kinase by capillary electrophoresis. T4 DNA polymerase efficiently fills in 5 or 27 nt gaps at 37° C. in NEBuffer 2 with no detectable strand-displacement activity (middle). The efficiency of T4 DNA polymerase filling in 27 nt gaps at room temperature, however, is significantly lower (bottom).

FIG. 17 shows the distance of mutant duplex bases from closest DNA fragment end for cfDNA collected from healthy donors and cancer patients as well as gDNA from FFPE tumor biopsies. Samples underwent either conventional ER/AT or Duplex-Repair.

FIG. 18 shows the characterization of the activity of Klenow fragment (exo-) and Taq DNA polymerase by capillary electrophoresis. Klenow (exo-) and Taq DNA polymerase efficiently perform dA-tailing with only dATP present at concentrations of 0.2 mM (middle) or 2 mM (bottom).

FIG. 19 shows the characterization of the activity of T4 DNA ligase and 5′ deadenylase by BioAnalyzer. T4 DNA ligase and 5′ deadenylase efficiently ligate NGS adapters to a 166 bp blunted duplex with dA tails in the presence of 15 (top) or 20% (bottom) weight by volume (w/v) PEG 8000. To minimize spurious intermolecular ligation at high PEG concentrations, Duplex-Repair only uses 10% w/v PEG 8000 during adapter ligation. Of note: the unit of the x axis of the top panel could not be converted to bp by BioAnalyzer software.

FIG. 20 shows the characterization of the combined efficiency of dA-tailing and adapter ligation by BioAnalyzer. The combined efficiency of dA-tailing and adapter ligation of Duplex-Repair could be higher than that of the Kapa Hyper kit. The input was a 274 bp blunted duplex. Of note, the unit of the x axis of the top panel could not be converted to bp by BioAnalyzer software.

FIG. 21 shows the characterization of the performance of Duplex-Repair (after optimizing reaction conditions and eliminating multiple Ampure cleanups) by capillary electrophoresis. Duplex-Repair facilitates the formation of a major product of NGS adapter-ligated oligonucleotides that are ready for sequencing applications. The ‘nick sealing products’ (middle) were collected following steps 1-3 of duplex repair but prior to dA-tailing. The ‘adapter ligated products’ (bottom) have undergone the entire Duplex-Repair protocol and ligation to NGS adapters, which add an additional 39-40 or 37-38 bp (unique molecular indices can be either 3 or 4 base pairs) to the exposed 3′ and 5′ ends of oligonucleotides after Duplex-Repair respectively (note: adapters in schematic not drawn to scale).

FIG. 22 shows characterization of the performance of Duplex-Repair (after optimizing reaction conditions and eliminating multiple Ampure cleanups) as a function of DNA input mass by capillary electrophoresis. Duplex-Repair is effective at preparing cfDNA inputs ranging from 20 to 200 ng for NGS. The ‘nick sealing products’ (top rows) were collected following steps 1-3 of duplex repair but prior to dA-tailing. The ‘adapter ligated products’ (bottom rows) have undergone the entire Duplex-Repair protocol and ligation to NGS adapters, which add an additional 39-40 or 37-38 bp (unique molecular indices can be either 3 or 4 base pairs) to the exposed 3′ and 5′ ends of oligonucleotides after Duplex-Repair respectively.

FIGS. 23A-23D show the quantification of strand resynthesis using Single-Molecule Real-Time (SMRT) sequencing. FIG. 23A shows a schematic of library construction for PacBio SMRT sequencing using modified dNTPs to aid in identifying resynthesis regions. FIG. 23B shows the estimated fractions of interior base pairs (>12 bp from either end of the original duplex fragment) that were resynthesized using conventional ER/AT and several variations of Duplex-Repair. FIG. 23C shows the observed average interpulse durations (IPD; in frames) for circular consensus sequence (CCS) read strands relative to the distance from the original 3′ end of those strands across three sample types. FIG. 23D shows the estimated fraction of interior base pairs resynthesized for both conventional ER/AT and Duplex-Repair across three sample types.

FIG. 24 shows background estimated resynthesis of interior base pairs using standard dNTPs across FFPE and cfDNA sample types.

FIG. 25 shows characterization of the activity of DNase 1 by BioAnalyzer. The input was a 100 bp dsDNA oligo. The results show that up until 20 mU of DNase 1, the dominant fragment length is still 100 bp.

FIG. 26 shows the characterization of the activity of DNase 1 by capillary electrophoresis. For all concentrations of DNAse 1 tested, the major product as determined by capillary electrophoresis is the 100mer duplex. However, intermediate-sized fragments (shown in boxes) are detected with 2 and 20 mU of DNase 1, suggesting that ≥2 mU of DNase 1 nick but do not significantly degrade dsDNA. These intermediate-sized fragments are present in capillary electrophoresis traces, as heat pretreatment and denaturation is required, but not on BioAnalyzer traces in which there is no denaturation (FIG. 24).

FIG. 27 shows characterization of the oxidation activity of CuCl₂/H₂O₂ by Sanger sequencing. The input was a 274 bp dsDNA oligo and was treated with different concentrations of CuCl₂/H₂O₂. The dashed boxes indicate where C->A mutations are detected when treated with 1000 μM CuCl₂/H₂O₂. SEQ ID NO: 34 is shown.

FIGS. 28A-28D show targeted panel sequencing of cfDNA and FFPE tumor biopsies. FIG. 28A shows measured duplex sequencing error rates of HD_78 cfDNA damaged with varied concentrations of DNase I (to induce nicks) and CuCl₂/H₂O₂ (to induce oxidative damage) and then repaired by using Duplex-Repair or conventional ER/AT (three replicates per condition). FIG. 28B shows duplex sequencing error rates of four healthy cfDNA samples (three replicates per condition), three cancer patient cfDNA samples (one replicate per condition), and five cancer patient FFPE tumor biopsies (three replicates per condition) treated with conventional ER/AT or Duplex-Repair. FIG. 28C shows aggregate mutant bases and their position relative to the end of the original duplex fragment. Dashed line represents the threshold of the interior of the fragment (12 bp). FIG. 28D shows error rates from FIG. 28B compared to their corresponding estimates of interior base pair resynthesis fractions from FIG. 23D. Pearson's correlation calculated for all data points.

FIG. 29 shows error rates by mutation context observed in healthy donor cfDNA treated with varied concentrations of CuCl₂/H₂O₂ and DNase I.

FIG. 30 shows error rates by mutation context observed in duplex sequencing of a pan-cancer panel for cfDNA samples and FFPE tumor biopsies treated with conventional ER/AT vs. Duplex-Repair.

FIGS. 31A-31D shows targeted panel sequencing of cfDNA and FFPE tumor biopsies. FIG. 31A shows measured duplex sequencing error rates of HD 78 cfDNA damaged with varied concentrations of DNase I (to induce nicks) and CuCl₂/H₂O₂ (to induce oxidative damage) and then repaired by using Duplex-Repair or conventional ER/AT (three replicates per condition). FIG. 31B shows background errors in pan-cancer panel duplex sequencing of a heavily damaged cfDNA sample (2mU DNase I, 100 μM CuCl₂/H₂O₂) subjected to conventional ER/AT versus Duplex-Repair, normalized for the same number of evaluable duplexes (DSCs). FIGS. 31C-31D show duplex sequencing error rates for cancer patient cfDNA samples (one replicate per condition, FIG. 31C) and five FFPE tumor biopsies (three replicates per condition, FIG. 31D) treated with Duplex-Repair vs. conventional ER/AT.

FIGS. 32A-32F: Duplex-Repair reduces strand resynthesis and improves sequencing accuracy. FIG. 32A shows the estimated fractions of interior base pairs (>12 bp from either end of the original duplex fragment) that were resynthesized using conventional ER/AT and several variations of Duplex-Repair, as measured using a custom single-molecule sequencing assay. FIG. 32B shows the estimated fraction of interior base pairs resynthesized for both conventional ER/AT and Duplex-Repair across three sample types. FIG. 32C shows duplex sequencing error rates of four healthy cfDNA samples (three replicates per condition), three cancer patient cfDNA samples (one replicate per condition), and five cancer patient FFPE tumor biopsies (three replicates per condition) treated with conventional ER/AT or Duplex-Repair. FIG. 32D shows aggregate mutant bases and their position relative to the end of the original duplex fragment. Dashed line represents the threshold of the interior of the fragment (12 bp). FIG. 32E shows measured duplex sequencing error rates of HD 78 cfDNA damaged with varied concentrations of DNase I (to induce nicks) and CuCl₂/H₂O₂ (to induce oxidative damage) and then repaired by using Duplex-Repair or conventional ER/AT (three replicates per condition). FIG. 32F shows a comparison of conventional ER/AT and Duplex-Repair for cfDNA and FFPE sample types shows comparable duplex recoveries as a function of the number of read pairs, as analyzed via in silico downsampling of reads.

FIGS. 33A-33C: FIG. 33A shows an overview of Duplex-Repair and Duplex-Repair ‘v2’ (e.g., an alternative method of Duplex Repair) as compared to conventional ER/AT methods. FIG. 33B shows a schematic of the major products of various synthetic duplexes subjected to each step of Duplex-Repair and conventional ER/AT as determined by capillary electrophoresis. The non-fluorophore-tagged ends of the synthetic molecules are depicted, and fragment sizes are drawn to scale. Duplexes demarcated by asterisks (*) do not contain fluorophores and were not directly observed by capillary electrophoresis; however, their presence is predicted due to the characterized activities of UDG and FPG. Regions of strand resynthesis are illustrated by dashed lines. FIG. 33C shows the measured library conversion efficiencies of Duplex-Repair vs. the KAPA™ HyperPrep kit as a function of DNA input by using a ddPCR assay.

FIG. 34 shows a step-by-step comparison between convention ER/AT repair, with NEB PRECR® pretreatment (left column), and Duplex-Repair (DR) ER/AT (right column).

FIGS. 35A-35C provides a description of the structures (FIG. 35A) associated with each step of conventional ER/AT (with optional pretreatment by NEB PRECR® and/or ExoVII) versus Duplex-Repair (DR) ER/AT. The details of the enzyme compositions and activities at each of steps (i) through (vii) are provided in FIG. 35B for convention ER/AT (with optional pretreatment by NEB PRECR® and/or ExoVII) and in FIG. 35C for Duplex-Repair.

FIG. 36 shows the characterization of the activity of HiFi Taq DNA ligase by capillary electrophoresis. HiFi Taq DNA ligase efficiently seals nicks in NEBuffer 2 and HiFi Taq ligase buffer mix (bottom) as it does in HiFi Taq ligase buffer alone (middle).

FIGS. 37A-37D show the quantification of resynthesized bases with conventional ER/AT applied to cfDNA and FFPE tumor biopsies. IPD signals when conventional ER/AT with either standard dNTPs or modified dNTPs was applied to healthy cfDNA (FIG. 37A) and FFPE tumor biopsies (FIG. 37B), and corresponding estimates of number of bases filled in during ER/AT for healthy cfDNA (FIG. 37C) and FFPE tumor biopsies (FIG. 37D).

### DETAILED DESCRIPTION

Improving the accuracy of next generation sequencing (NGS) is a significant goal in clinical medicine. This is particularly important when seeking to detect low-abundance mutations in clinical specimens, such as for early cancer detection (Chabon et al., Nature, 2020; Corcoran et al., Ann Rev Cancer Bio, 2019), monitoring of minimal residual disease (“MRD”) (Parsons et al., Clinic Cancer Res, 2020; Tie et al., Sci Trans Med, 2016), tracing of actionable or resistance mutations (Parikh et al., Nat Med, 2019), performing prenatal genetic tests (Lo et al., Sci Trans Med, 2010) and detecting microbial or viral infections (Blauwkamp et al., 2019), as errors could lead to incorrect diagnoses and treatments. DNA base damage is a major source of false mutation discovery in NGS (Chen et al., Science, 2017). Lesions such as cytosine deamination, thymine dimers, pyrimidine dimers, 8-Oxoguanine, 6-O-methylguanine, depurination, and depyrimidination arise both spontaneously and in response to environmental and chemical exposures such as ultraviolet (UV) radiation, ionization radiation, reactive oxygen species, and genotoxic agents, or sample processing procedures, such as formalin fixation, freezing and thawing, heating, acoustic shearing, and long-term storage in aqueous solution (Costello et al., Nucleic Acids Res, 2013; Wong et al., BMC Med Genomics, 2014). If left uncorrected, such lesions could result in altered base pairing when copied by a polymerase capable of translesion synthesis, thereby leading to detection of a false mutation. These problems, along with other errors introduced in library amplification and sequencing, contribute to an error rate of 0.1%-1% in standard NGS (Salk et al., Nat Rev Genetics, 2018).

Due to the stochasticity of base damage errors, many can be overcome by sequencing multiple copies of each DNA fragment and requiring a consensus among reads. Such “consensus-based” sequencing can reduce errors by up to 100-fold, when requiring a consensus from each single strand of DNA, and up to 1000-fold, when requiring a consensus from both sense strands of each DNA duplex.

Methods requiring the sequencing and reading of both sense strands of a duplex are known as “duplex sequencing” (Schmitt et al., PNAS, 2012). However, existing methods for ‘end repair/dA-tailing’ (ER/AT) which are used to correct backbone damages (e.g., nicks, gaps, and overhangs) in duplex DNA, and facilitate ligation of NGS adapters, could resynthesize portions of each duplex prior to adapter ligation. If resynthesis occurs in the presence of base damage, translesion synthesis could copy errors to both strands and render them indistinguishable from true mutations on both strands.

This major source of false discovery in duplex sequencing is most clearly seen at fragment ends where short 5′ overhangs are often filled in. Yet, this could also span much deeper given (i) the 5′ exonuclease and strand-displacement activities of Taq and Klenow polymerases used in ER/AT, and (ii) the varied backbone damages that could act as ‘priming sites’ for strand resynthesis.

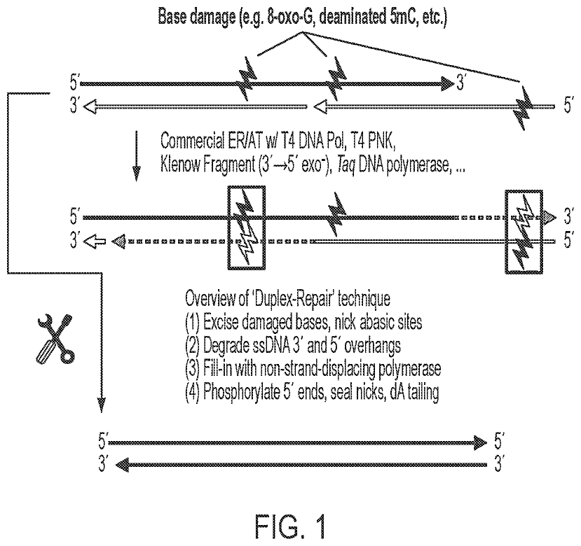

Disclosed herein is a workflow approach called Duplex-Repair which limits the potential for base damage errors to be copied to both strands by, in part, minimizing polymerization prior to NGS adapter ligation to dramatically reduce duplex sequencing error rates (e.g., see FIG. 1).

Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention. Accordingly, the terms defined immediately below are more fully defined by reference to the specification as a whole.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 3D ED., John Wiley and Sons, New York (2006), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

The term “mutation,” as may be used herein, refers to a change, alteration, or modification to a nucleotide in a nucleic acid as compared to its wild-type sequence. For example, without limitation, mutations may include substitutions, insertions, deletions, or any combination of the same. In some embodiments, there is at least one mutation. In some embodiments, there are more than one mutation. In some embodiments, where there is more than one mutation, the mutations are distinct (e.g., not of the same type (e.g., substitutions, insertions, deletions)). In some embodiments, where there is more than one mutation, the mutations are the same (e.g., of the same type (e.g., substitutions, insertions, deletions)). Additionally, in some embodiments, the mutations result in a frameshift. The terms “wild type” and “native,” as may be used interchangeably herein, are terms of art understood by skilled artisans and mean the typical form of an item, organism, strain, gene, or characteristic as it occurs in nature as distinguished from engineered, mutant, or variant forms.

The terms “nucleic acid,” “nucleotide sequence,” “polynucleotide,” “oligonucleotide,” and “polymer of nucleotides,” as may be used interchangeably herein, refer to a string of at least two, nucleobase-sugar-phosphate combinations (e.g., nucleotides) and includes, among others, single-stranded and double-stranded DNA, DNA that is a mixture of single-stranded and double-stranded regions, single-stranded and double-stranded RNA, and RNA that is mixture of single-stranded and double-stranded regions, hybrid molecules comprising DNA and RNA that may be single-stranded or, more typically, double-stranded or a mixture of single-stranded and double-stranded regions. In addition, the terms (e.g., nucleic acid, et al.) as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often referred to as an oligonucleotide.

The terms (e.g., nucleic acid, et al.) also encompass such chemically, enzymatically, or metabolically modified forms of nucleic acids, as well as the chemical forms of DNA and RNA characteristic of viruses and cells, including simple and complex cells. For instance, the terms (e.g., nucleic acid, et al.) as used herein can include DNA or RNA as described herein that contain one or more modified bases. The nucleic acids may also include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, 0(6) methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages). Thus, DNA or RNA including unusual bases, such as inosine, or modified bases, such as tritylated bases, to name just two examples, are nucleic acids as the term is used herein. The terms (e.g., nucleic acid, et al.) also includes peptide nucleic acids (PNAs), phosphorothioates, and other variants of the phosphate backbone of native nucleic acids. Natural nucleic acids have a phosphate backbone, artificial nucleic acids can contain other types of backbones, but contain the same bases. Thus, DNA or RNA with backbones modified for stability or for other reasons are nucleic acids as that term is intended herein.

The term “nucleobase,” as may be used herein, is a term of art known to the skilled artisan as a nitrogenous base, which is a nitrogen-containing biological compound that forms a component of a nucleoside, which is itself a component of a nucleotide. The nucleobases (also referred to herein as simply a base), are one of the basic building blocks of nucleic acids (e.g., DNA, RNA) as they possess the ability to form base pairs and to stack one upon another and forming the long-chain helical structures. There are five canonical nucleobases: adenine (A), cytosine (C), guanine (G), thymine (T), and uracil (U), with A, C, G, and T being found in DNA and A, C, G, and U being found in RNA.

The term “nucleoside,” as may be used herein, refers to glycosylamines (e.g., N-glycosides) that are generally known to be nucleotides without a phosphate group. A nucleoside consists of a nucleobase (e.g., a nitrogenous base) and a five-carbon sugar (e.g., pentose). The five-carbon sugar can be either ribose or deoxyribose. Nucleosides are the biochemical precursors of nucleotides, which are the constituent components of RNA and DNA. Examples of nucleosides include cytidine (C), uridine (U), adenosine (A), guanosine (G), thymidine (T), and inosine (I), but includes variants (e.g., modified or synthetic nucleosides, nucleosides containing modified or synthetic nucleobases).

The term “nucleotide,” as may be used herein is a term of art known to the skilled artisan to generally refer to those compositions comprising a nucleobase, sugar, and phosphate (e.g., a nucleoside and a phosphate) (which compositions (e.g., nucleotides) are separated into purines and pyrimidines). Nucleotides are components of nucleic acids that can be copied using a polymerase. Nucleosides, cytidine (C), uridine (U), adenosine (A), guanosine (G), thymidine (T), and inosine (I), along with a phosphate group, represent the canonical nucleotides, and may be referred to in DNA form (e.g., with a deoxyribose) as dATP, dGTP, dCTP, and dTTP when referring to individual nucleotides used in a synthesis reaction (e.g., nucleotide with 3 phosphate groups (e.g., “tri-phosphate”)). Two of the phosphate groups may be hydrolyzed to yield a monophosphate nucleotide for use in the polymerization of a nucleic acid. Generally, dATP, dGTP, dCTP, and dTTP may be referred to as dNTPs, wherein “N” represents the ambiguity as to the nature of the nucleoside. Thus, a mixture of dNTPs may include a concentration of all or some of each. Nucleotides contain not only the known purine and pyrimidine bases, but also other heterocyclic bases that have been damaged (e.g., bases that have oxidized, methylated, acylated, deadenylated, etc.). The term is well-known in the art and will be readily appreciated by the skilled artisan.

DNA synthesis embraces both enzymatic-based (e.g., DNA polymerase based off a template strand) and chemical synthesis methods. In various embodiments, DNA synthesis refer to the enzymatic process, whereby a DNA polymerase creates a newly made strand of DNA based on catalyzing the successive joining of incoming nucleotide base pairs to an available 3′ end of a growing DNA strand through the formation of a new phosphodiester linkages between the terminal nucleotide of the growing strand and the incoming nucleotide base being added to the growing strand. Typically, the order of nucleotide bases added to the growing DNA chain is determined by the opposite strand of DNA through the hydrogen bond-based pairing with is cognate base pair on the “template” strand. DNA resynthesis refers to a form of DNA synthesis that typically occurs at a nick or a gap in one of the strands of a DNA double helix, such that an available 3′ end is exposed from which DNA synthesis occurs, and wherein the DNA polymerase concurrently displaces the downstream existing strand while synthesizing a new strand against the template strand.

The term “polymerase,” as may be used herein, is a term of art known to the skilled artisan to refer generally to an enzyme which aids in, or synthesizes nucleic acids (e.g., DNA polymerase, RNA polymerase) and polymers. There are known a multitude of polymerases, for example, without limitation and which are all contemplated herein, DNA polymerase I (Pol gamma, Pol theta, Pol nu), DNA polymerase II (Pol alpha, Pol delta, Pol epsilon, Pol zeta), DNA polymerase III holoenzyme, DNA polymerase IV (DinB) (SOS repair polymerase, Pol beta, Pol lambda, Pol mu), DNA polymerase V (SOS polymerase, Pol eta, Pol iota, Pol kappa), Reverse transcriptase, and RNA polymerase (RNA Pol I, RNA Pol II, RNA Pol III, T7 RNA Pol, RNA replicase, Primase). Additionally, as is further contemplated, are polymerases from bacterium (e.g., Thermus aquaticus). For example, Taq from Thermus aquaticus is a common DNA polymerase used in polymerase chain reactions (PCR). In some embodiments, a polymerase is a Taq polymerase. In some embodiments, a polymerase lacks 3′ to 5′ exonuclease activity. In some embodiments, a polymerase is a Klenow fragment. In some embodiments, a polymerase is a Klenow fragment lacking 3′ to 5′ exonuclease activity. In some embodiments, a polymerase is a human variant of any of the polymerases described herein.

The term “adapter ligation,” as may be used herein, refers to the term as known to the skilled artisan to generally refer to the process of attaching (e.g., ligating) known sequences of nucleotides (e.g., nucleic acids, oligonucleotides, e.g., adapters) to one or more ends of one or more nucleic acids (e.g., DNA fragments, complementary strands of DNA). Often adapters contain specific sequences which are complementary to the nucleic acid fragments they are intended to attach to, for example, without limitation in the event nucleic acids are dA-tailed, an adapter may have a “T” overhang, wherein the “T” refers to a nucleotide comprising a thymine nucleobase. The T overhang is complementary to the dA-tail, thus facilitating ligation.

The term “dA-tailing,” as may be used herein, refer to the status, or to a characteristic, of a nucleic acid (e.g., DNA, RNA) as having a “tail” comprising a non-templated adenosine (A) (e.g., adenosine monophosphates). By “tail” it is meant that the adenosines (e.g., AAAAA) at the 3′ end of the nucleic acid (e.g., DNA, RNA), comprises an overhang beyond the 5′ terminal nucleotide of the complementary strand. The term (e.g., dA-tail) may be used as a verb (e.g., dA-tailing) to describe the process by which the adenosine is added to the 3′ end of a nucleic acid. In some embodiments, dA-tailing is performed using Taq polymerase. In some embodiments, dA-tailing is performed using Klenow Fragment lacking 3′ to 5′ exonuclease activity.

The term “overhang,” as may be used herein, is a term of art known to the skilled artisan to refer to a portion of a double-stranded nucleic acid which extends (e.g., protrudes) beyond the end (e.g., terminal nucleotide) of the opposing strand (e.g., complementary strand). For example, without limitation, a 5′ overhang will refer to the portion of a strand of a nucleic acid which extends beyond the 3′ end (3′ terminal nucleotide) of the opposing strand (e.g., complementary strand) with which it forms a double-stranded nucleic acid duplex. As an additional example, without limitation, a 3′ overhang will refer to the portion of a strand of a nucleic acid which extends beyond the 5′ end (5′ terminal nucleotide) of the opposing strand (e.g., complementary strand) with which it forms a double-stranded nucleic acid duplex. As will be appreciated by the skilled artisan, a double-stranded duplex, may comprise both a 5′ and 3′ overhang, a single 5′ overhang, two 5′ overhangs, a single 3′ overhang, two 3′ overhangs, an overhang (e.g., 5′ or 3′) and a blunt end, or two blunt ends. As used herein, the term “blunt end,” refers the quality of double-stranded duplex, wherein the two strands forming the duplex terminate at the same pair of nucleotides and thus has no overhang at that end of the duplex (e.g., the end is blunt).

The term “exonuclease,” as may be used herein, refers to the term of art generally known to the skilled artisan to refer to an enzyme that has at least the activity of cleaving nucleotides from the end of a nucleic acid (e.g., polynucleotide, oligonucleotide). In some embodiments, an exonuclease will cleave the nucleotides one at a time. An exonuclease may cleave nucleotides in either direction (e.g., from either the 5′ or 3′ end) of a nucleic acid. When describing such activity, often the notation is shown to be 5′ to 3′ exonuclease activity, when referring to an exonuclease that cleaves nucleotides starting from the 5′ end of a nucleic acid (e.g., the 5′ nucleotide which is distal to the 3′ end) or 3′ to 5′ exonuclease activity, when referring to an exonuclease that cleaves nucleotides starting from the 3′ end of a nucleic acid (e.g., the 3′ nucleotide which is distal to the 5′ end). In some embodiments, an exonuclease has 5′ to 3′ exonuclease activity. In some embodiments, the exonuclease can be Exo VII.

The terms “complementary” and “complementarity,” as may be used interchangeably herein, refer a property of a nucleotide (e.g., A, C, G, T, U) in a nucleic acid (e.g., RNA, DNA) in a strand (e.g., oligonucleotide) to pair with another particular nucleotide in a nucleic acid strand of the opposite orientation (e.g., strands running parallel, but in the reverse direction (i.e., 5′-3′ aligns with 3′-5′, and 3′-5′ with 5′-3′)) (i.e., Watson-Crick base-pairing rules). With respect to deoxyribonucleic acids (DNA) the base pairings which are complementary are adenine (A) and thymine (T) (e.g., A with T, T with A) and guanine (G) and Cytosine (C) (e.g., G with C, C with G) and with respect to ribonucleic acid (RNA) the base pairings which are complementary are A and uracil (U) (e.g., A with U, U with A) and G and C (e.g., G with C, C with G). This occurs because of the ability of each base pair to form an equivalent number of hydrogen bonds with its complementary base (e.g., A-T/U, T/U-A, C-G, G-C), for example the bond between guanine and cytosine shares three hydrogen bonds compared to the A-T/U bond which always shares two hydrogen bonds.

When every base in at least one strand of a pair of nucleic acids is found opposite its complementary base pair, such strand is considered fully complementary to its sequence in the other strand. When one, or more, bases of such a strand is found in a position where it is opposite any other base excepting its complementary base pair, that base is considered “mis-matched” and the strand is considered partially complementary. Accordingly, strands can be varying degrees of partially complementary, until no bases align, at which point they are non-complementary.

Other non-standard nucleotides (e.g., 5-methylcytosine, 5-hydroxymethylcytosine) are known in the art and their properties and complementarity will be readily apparent to the skilled artisan.

Duplex-Repair can ensure high accuracy sequencing even when there is extensive DNA damage in a sample. Here, dramatic error reductions were observed both in a heavily damaged cfDNA sample and a FFPE gDNA sample, although the error rates of the FFPE gDNA sample repaired by Duplex-Repair were still slightly higher than those of the cfDNA sample. Considering that base and backbone damage can arise spontaneously and in response to environmental and chemical agents, Duplex-Repair is needed to ensure the reliability of duplex sequencing for a wide range of samples.

Resynthesis was still needed within gap regions and short (≤7 nt) remaining 5′ overhangs after the DNA lesion repair and overhang removal step, as ExoVII could not fully blunt 5′ overhangs. However, restricting fill-in to gap regions protected against error propagation while ensuring maximum duplex recovery. Furthermore, by limiting the lengths of 5′ overhangs filled in during ER/AT, it was possible to concentrate end repair errors within fragment ends and filter against them in silico by their distance from fragment ends. Additionally, the enzyme cocktail used in the DNA lesion repair and overhang removal step only recognized the most prevalent of DNA base lesions, while there are a large number of possible base damages (Cadet and Wagner 2013) that can arise in DNA and lead to base mispairing. However, if they happen to occur in a duplex region where no DNA polymerization occurs or the polymerase(s) is incapable of translesion synthesis, it would not manifest as duplex sequencing errors but could result in losses of DNA duplexes.

The term “gap,” as may be used herein, refers to the term of art generally known to the skilled artisan to refer to the portion of a double-stranded nucleic acid duplex (e.g., a nucleic acid comprised at least two strands of nucleic acid with enough complementarity to form a duplex) which is single-stranded and which is bounded on each side by double-stranded portions. This “gap” between the double-stranded portions comprises a single-stranded portion of at least one (e.g., 1, 2, 3, 4, 5, 6, 7, 8, 9, 10, 11, 12, 13, 14, 15, 16, 17, 18, 19, 20, 21, 22, 23, 24, 25, or more) nucleotide which do not have at least one (e.g., 1, 2, 3, 4, 5, 6, 7, 8, 9, 10, 11, 12, 13, 14, 15, 16, 17, 18, 19, 20, 21, 22, 23, 24, 25, or more) nucleoside, and/or phosphate, opposite them. This term is contrasted with the term “nick” (as is further defined hereinbelow) in that a portion of the opposing strand (e.g., complementary strand) is absent in a gap, wherein with a nick, a portion of the strand may not be joined to an adjacent nucleotide, but they are all present in the opposing strand (e.g., complementary strand).

The term “nick,” as may be used herein, refers to the term of art generally known to the skilled artisan to refer to the portion of a double-stranded nucleic acid duplex (e.g., a nucleic acid comprised at least two strands of nucleic acid with enough complementarity to form a duplex) where there is a lack of bonding between two adjacent components of the strand. For example, without limitation, a nick may be described as a lack of continuity (e.g., discontinuity) between two adjacent nucleotides in one of the strands of a duplex. Nicks may form from a variety of causes and can be useful and detrimental to DNA carrying out its function. This term is contrasted with the term “gap” (as is further defined hereinabove) in that a portion of the opposing strand (e.g., complementary strand) is not absent in a nick wherein a portion of the strand may not be joined to an adjacent nucleotide, but they are all present in the opposing strand (e.g., complementary strand), whereas with a gap, a portion (e.g., nucleoside, phosphate group) of the opposing strand (e.g., complementary strand) is missing.

Disclosed herein, is a new ER/AT method called Duplex-Repair (DR), which minimizes and/or eliminates many of the problems inherent to existing methods. For example, without limitation, DR minimizes strand resynthesis prior to ligation of NGS adapters, which significantly limits false mutation discovery. As will be seen herein, by minimizing this resynthesis, DR addresses a major Achilles' heel of duplex sequencing, and other related methods which rely upon a consensus of sequences from both strands of each duplex, to provide maximum accuracy and robustness.

Mutations, which as described hereinabove, are regions (e.g., sections, portions, nucleobases, nucleosides, nucleotides) of a given nucleic acid (e.g., DNA, RNA) which differ as compared to their wild-type nucleic acid, will most often be reflected in each strand of a nucleic acid. That is to say that, when a mutation is present in a sample it and its complement will be observed in each strand of the nucleic acid when sequenced. This presents a problem however, when considering that a sample may contain single-stranded portions (e.g., gaps, overhangs), or areas which may instigate strand resynthesis (e.g., nicks). This problem presents because if a damaged base is present in such single-stranded region, or other region which is resynthesized, a damaged base may instruct the synthesis of its complementary strand to include a base which was not originally present in the nucleic acid from which the sample was generated (because damaged bases can affect non-canonical base pairings). The same could happen if one strand contains mismatched bases. In such instances, the mismatch will show a paired match in the re-synthesized complement instead of its native mismatched base. When this happens, a sequencing of both strands will read a mutation in each of the strands, thus show a mutation; however, this mutation may not be a true reflection of the original nucleic acid. Such mutations are termed “false mutations,” herein. False mutations are mutations which result from the resynthesis of complementary strands of nucleic acid, which do not represent the original (e.g., native, wild-type) complementary strand of nucleic acid from which the sample was obtained.

Accordingly, in some aspects, the disclosure relates to a method of preparing a nucleic acid sample (sample) for sequencing that minimizes propagation of false mutations due to amplification of nucleotide damage or alterations originally confined to one strand, wherein at least a portion of the sample is double-stranded, comprising adding a sample to a reaction vessel and: (a) contacting the sample to one or more enzymes capable of: (i) excising one or more damaged bases from the sample; (ii) cleaving one or more abasic sites, and processing the resulting ends to be compatible with extension by a DNA polymerase and/or ligation by a DNA ligase; and (iii) digesting 5′ overhangs; (b) contacting the sample with one or more of: (i) a DNA-dependent DNA polymerase lacking both strand displacement and 5′ exonuclease activities but capable of filling in single-stranded segments of the sample and digesting 3′ overhangs of the sample; and (ii) an enzyme capable of phosphorylating the 5′ ends of the strands of the sample; (c) contacting the sample with a DNA ligase capable of sealing nicks; and (d) preparing the sample for adapter ligation, wherein the preparing comprises adding dAMP to the 3′ ends of the strands of the sample (dA-tailing).

The term “reaction vessel,” as may be used herein, refers to a container which is used to carry out the reactions (e.g., methods) described herein. As will be appreciated by one of ordinary skill in the art, a reaction vessel will be one that is appropriate for the reaction or method to be performed therein. For instance, materials may be used such as plastics, (polyethylene, etc.), glass, metal, or other appropriate material, which are not degraded or susceptible to damage from the reagents (e.g., nucleic acids, dNTPs, enzymes) used therein (e.g., components of the methods as described herein). Examples of reaction vessels may be 96-well plates (or any other number of premade well plates), Eppendorf tubes, flasks, beakers, cylinders, and the like. Determination and selection of an appropriate reaction vessel will be immediately apparent to the skilled artisan and will not require undue experimentation.

The term “ligase,” as may be used herein, refers to the term of art generally known to the skilled artisan to refer to an enzyme that has at least the activity of catalyzing the joining of two molecules (e.g., nucleotides, e.g., sugar and phosphate groups of nucleotides) through the formation of a chemical bond. For example, without limitation, a ligase may join nucleotides through the formation of a phosphodiester bond (e.g., DNA ligase (e.g., DNA Ligase 1; NCBI RefSeqGene NG_007395.1; Taq DNA ligase (e.g., HiFi Taq DNA ligase; New England BioLabs, Inc.: neb.com/products/m0647-hi-fi-taq-dna-ligase #Product %20Information). Ligases may have varied final activities which employ the basis activity recited herein above (e.g., catalyzing the joining of two molecules), for example, without limitation, they may seal nicks and/or permit end joining (e.g., ligate two non-associated nucleic acids such as those not associated with the same nucleic acid duplex). Ligases are well known in the art and will be readily appreciated by the skilled artisan. In some embodiments, a ligase has nick sealing activity. In some embodiments, a ligase does not have (e.g., lacks) end joining activity. In some embodiments, a ligase has nick sealing activity, but lacks end joining activity. In some embodiments, a ligase is a DNA ligase. In some embodiments, a ligase is DNA ligase 1. In some embodiments, a ligase is a HiFi Taq ligase. In some embodiments, a ligase is a human ligase.

The term “lyase,” as may be used herein, refers to the term of art generally known to the skilled artisan to refer to an enzyme that has at least the activity of catalyzing the breaking of chemical bonds. However, lyases differ from other enzymes sharing similar activity in that lyases perform this breaking by means other than hydrolysis (e.g., a substitution reaction, addition reactions, and elimination reactions). Lyase-catalyzed reactions are known to often act by breaking the bond between a carbon atom and another atom (e.g., oxygen, sulfur, or another carbon atom). It is generally known that specific types of lyase exist in the field, and selection and use of the same will be readily apparent to the skilled artisan upon reading the instant disclosure. For, example without limitation, in some embodiments, a lyase is an AP lyase (e.g., DNA-AP-lyase). AP lyases art generally known in the art to facilitate the cleavage of C_{3′}-O—P bond 3′ from an abasic (e.g., apurinic or apyrimidinic) site in a nucleic acid via a beta-elimination reaction. This reaction leaves a 3′-terminal unsaturated sugar and a product with a terminal 5′-phosphate.

The term “damaged,” as may be used herein, when used in the context of describing a nucleobase, nucleoside, nucleotide, or nucleic acid, refers to any of these components being altered or modified from its natural state by degradative interactions with a substance or environmental factor. For example, damaged bases may refer to, without limitation, an oxidized base such as 8′-oxoguanine, a deaminated base (e.g., uracil which is produced by deamination of cytosine, or hypoxanthine (e.g., as found in inosine) which is produced by deamination of adenine), an oxidized pyrimidine, and/or a cyclobutane pyrimidine dimer. Damaged bases (e.g., DNA lesions) are well-known in the art and can result in errant or non-canonical base pairings (e.g., base pairings other than A/T, C/G, A/U). Further, the term (e.g., damaged), shall be understood to include abasic sites. Abasic sites are known in the art to generally refer to sites in a nucleic acid (e.g., DNA, RNA) where neither a purine or pyrimidine is found (e.g., the nucleotide is neither a pyrimidine nor purine). Abasic sites can arise wherein the sugar-phosphate backbone of DNA is intact, but where the nucleobase itself is missing.

Duplex Sequencing

Duplex sequencing is a type of nucleic acid sequencing which uses the information from both strands of a duplex to generate results regarding the genomic profile of a sample, or subject from which a sample was obtained. The term “subject,” as used herein, refers to any organism in need of treatment or diagnosis using the subject matter herein. For example, without limitation, subjects may include mammals and non-mammals. In some embodiments, a subject is mammalian. In some embodiments, a subject is non-mammalian. As used herein, a “mammal,” refers to any animal constituting the class Mammalia (e.g., a human, mouse, rat, cat, dog, sheep, rabbit, horse, cow, goat, pig, guinea pig, hamster, chicken, turkey, or a non-human primate (e.g., Marmoset, Macaque)). In some embodiments, a mammal is a human. The term “duplex sequencing,” as used herein, also embodies any sequencing method which derives high accuracy by requiring a consensus of sequences from both strands of each DNA duplex. Duplex sequencing inherently possesses the ability to provide greater accuracy regarding the sequence of the nucleic acid, as computational analysis can resolve errors by using known properties of a duplex. For example, without limitation, the understanding that nucleobases form canonical base “pairings” when part of a duplex. This property of nucleic acids has been well-known since at least the later half of the past century, and is readily understood and appreciated by those in the art. Accordingly, employing this knowledge, it is possible to infer and determine the predicted complementary sequence from the sequencing of one strand of a duplex. This inferred complementary sequence can then be compared with the results from the sequenced second strand of nucleic acid of the duplex. When such two strands are compared, they can confirm the sequences obtained, or highlight differences, thus pinpointing possible lesions (e.g., damaged bases) or mismatches only found on one strand, or sequencing errors or areas for further investigation. These differences may result from errant base insertions, deletions, or mutations (e.g., damaged bases). Further, the results of sequenced duplexes can further be compared to reference data further providing insight into possible mutations in the sequence. Accordingly, duplex sequencing provides for a high-accuracy method of resolving the sequence of nucleic acids, which accuracy permits greater resolution in determining the effect of differences therein (e.g., the effect of mutations in the genomic data).

Duplex sequencing requires many of the same steps as traditional sequencing. One step of particular interest is manipulating the sample duplex such that the strands are substantially “duplexed,” meaning that they consist of two strands of nucleic acids which are free from single-stranded portions (e.g., gaps, overhangs) and continuous (e.g., lacking nicks). Additionally, the strands must be prepared for ligation of adapters used in the sequencing process. Traditionally, this process uses a number of specific enzymes such as DNA polymerase(s) to primarily digest 3′ overhangs and fill-in 5′ overhangs, polynucleotide kinase(s) to phosphorylate fragment ends, and DNA polymerase(s) to perform non-templated addition of adenine (e.g., in the form of deoxyadenosine monophosphate (dAMP) to 3′ ends (e.g., when the ligation of deoxythymine monophosphate (dTMP)-tailed sequencing adapters is sought). For example, DNA polymerase(s) are provided along with a mixture of dNTPs to initiate synthesis of strands where a 3′ terminal nucleotide is recognized and there is a corresponding template strand. This site (e.g., 3′ terminal nucleotide) may be at a nick, gap, or on the 3′ end of a strand where the duplex contains a 5′ overhang. Further, because one or more of the DNA polymerase(s) used has either strand displacement or 5′ exonuclease activity, it will remove (e.g., displace or digest) any downstream fragment. For example, without limitation, in the instance the synthesis is initiated at a nick or gap, the newly synthesized strand will remove the downstream ‘native’ strand and re-synthesize it. This resynthesis, while correcting some of the issues mentioned, is not fail-safe, and can introduce errant information into the re-synthesized strand which were not present in the original ‘native’ strand. This can occur as a result of synthesis over a mismatched or damaged base (e.g., lesion), which may instruct the polymerase to insert a base that is complementary to the mismatched or damaged base, which was not representative of the base in the ‘native’ strand. This will then be interpreted in the results from sequencing as a correctly paired set of bases on both strands, as opposed to a mismatched base on one strand, which is not accurate (e.g., is a false mutation). This same error may appear any place synthesis occurs over a damaged or mismatched base (e.g., in instances where the sample is single-stranded as well). Additionally, such strand displacement and re-synthesis may cover (e.g., erase) disagreements in the strands, or places in the duplex where there is a mismatch. Accordingly, improvements are needed to increase the accuracy of duplex sequencing methods and to mitigate the introduction of false mutations.

The term “substantially,” as may be used herein, when used to describe the degree or abundance of an activity, generally refers to the value of the activity as being an amount which is achievable without undue effort. As can be appreciated, this amount may vary depending on the activity being performed, with simpler activities requiring a higher threshold and more complex activities requiring a lower threshold. For example, without limitation, when referring to substantially eliminating or removing reagents, dNTPs, or enzymes from a mixture, a substantial amount, may refer to 50% or more removal. In some embodiments, substantial refers to at least 50% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 99.95%, 99.99%, or more) and to all values of the variable that are within the experimental error (e.g., within the 95% confidence interval for the mean) or within +/−10% of the indicated value, whichever is greater. In some embodiments, substantially refers to at least 75% of the target being removed. In some embodiments, substantially refers to at least 80% of the target being removed. In some embodiments, substantially refers to at least 85% of the target being removed. In some embodiments, substantially refers to at least 90% of the target being removed. In some embodiments, substantially refers to at least 95% of the target being removed.

The term “kinase,” as may be used herein, is a term of art known to the skilled artisan to refer to an enzyme that catalyzes the transfer of a phosphate group to a substrate (e.g., phosphate group from ATP to a nucleic acid (e.g., DNA)). Accordingly, kinases may be used to prepare DNA for ligation (e.g. by ensuring that a 5′ phosphate is available). In some embodiments, a kinase is polynucleotide kinase (Pnk). In some embodiments, a kinase is a T4 polynucleotide kinase.

The term “downstream,” as may be used herein, refers to the location of a nucleotide in relation to a landmark in a given sequence of multiple nucleotides (e.g., a nucleic acid), such that downstream shall mean “more 3′” (in the case of a nucleic acid) than the landmark. For example, a nucleotide is downstream from a landmark if it is closer to the 3′ end (and thus further from the 5′ end) of the nucleic acid than the landmark. Conversely, the term “upstream,” as may be used herein, refers to the location of a nucleotide in relation to a landmark of a given sequence of multiple nucleotides (e.g., a nucleic acid), such that upstream shall mean “more 5′” (in the case of a nucleic acid) than the landmark. For example, a nucleotide is upstream from a landmark if it is closer to the 5′ end (and thus further from the 3′ end) of the nucleic acid than the landmark.

Duplex Repair (DR) Methods

Accordingly, in some aspects, the disclosure relates to a method of preparing a nucleic acid sample (sample; and as such term is further elaborated upon herein) for sequencing that minimizes propagation of false mutations due to amplification of nucleotide damage or alterations originally natively located in only one strand, wherein at least a portion of the sample is double-stranded, comprising adding a sample to a reaction vessel and: (a) contacting the sample to one or more enzymes capable of: (i) excising one or more damaged bases from the sample; (ii) cleaving one or more abasic sites, and processing the resulting ends to be compatible with extension by a DNA polymerase and ligation by a DNA ligase; (iii) and digesting 5′ overhangs; (b) contacting the sample with one or more of: (i) a DNA-dependent DNA polymerase lacking both strand displacement and 5′ exonuclease activity but capable of fill-in single-stranded segments of the sample and/or digesting 3′ overhangs of the sample; and (ii) an enzyme capable of phosphorylating the 5′ ends of the strands of the sample; and (c) contacting the sample with a DNA ligase capable of sealing nicks. In some embodiments, the methods of the present disclosure further comprise (d) preparing the sample for adapter ligation, wherein the preparing comprises: (i) adding deoxyadenosine monophosphate (dAMP) to the 3′ ends of the strands of the sample (dA-tailing); or (ii) optionally further blunting the ends of the sample.

In some aspects, a method comprises preparing a nucleic acid sample (sample) wherein at least a portion of the sample is double-stranded, comprising adding a sample to a reaction vessel and: (a) contacting the sample with one or more enzymes capable of: (i) phosphorylating the 5′ ends of the strands of the sample; adding a 3′ hydroxyl moiety to the 3′ ends of the strands of the sample; and (ii) sealing nicks; (b) contacting the sample with one or more of an enzyme capable of removing the 5′ and 3′ overhangs while also digesting gap regions to produce blunted duplexes; and (c) adding deoxyadenosine monophosphate (dAMP) to the 3′ ends of the strands of the sample (dA-tailing). In such a method, the need to excise damaged bases, to treat with ExoVII, or to fill gaps and short 5′ overhangs which were left after ExoVII treatment may be mitigated by the use of an enzyme (e.g., endonuclease (e.g., Nuclease S1)) to cleave single-stranded gap regions and cleave nucleotides present in overhang regions. In some embodiments, an enzyme used in step (a)(1) comprises: T4 polynucleotide kinase, HiFi Taq Ligase, or a combination thereof. In some embodiments, an enzyme used in step (b) is Nuclease S1.

The terms “endonuclease” and “nuclease,” as may be used herein, is a term of art known to the skilled artisan to refer generally to an enzyme that cleaves a phosphodiester bond or bonds within a polynucleotide chain (e.g., oligonucleotide, nucleic acid). Nucleases may be naturally occurring or genetically engineered. In some embodiments, an endonuclease is endonuclease IV (EndoIV). In some embodiments, an endonuclease is endonuclease VIII (EndoVIII). In some embodiments, a nuclease comprises Nuclease S1 (see for example, without limitation, thermofisher.com/order/catalog/product/EN0321#/EN0321; promega.com/products/cloning-and-dna-markers/molecular-biology-enzymes-and-reagents/s1-nuclease/?catNum=M5761; takarabio.com/products/cloning/modifying-enzymes/nucleases/s1-nuclease; and sigmaaldrich.com/US/en/product/SIGMA/N5661). Nuclease S1 degrades single-stranded nucleic acids, releasing 5′-phosphoryl mono- or oligonucleotides and may also cleave double-stranded DNA (dsDNA) at the single-stranded region caused by a nick, gap, mismatch, or loop.

By performing a method as described herein, the likelihood of the introduction of false mutations is substantially mitigated. For example, by using enzymes which first perform the excision of damaged bases and cleaving of abasic sites and processing of the resulting ends to be compatible with extension by a DNA polymerase and ligation by a DNA ligase from the sample, either the base will be excised in one strand and a gap will be created (where a complementary strand still exists at the excision point and forms a backbone for the duplex to remain intact), or a duplex/strand break will occur, thus creating two ‘daughter’ duplexes (where a complementary strand does not exist at the excision point and the duplex breaks apart into two smaller nucleic acids). A benefit, without limitation, of this step is to induce strand breaks in gap regions bearing damaged bases, as step (b) of the methods disclosed herein may involve using a DNA polymerase to fill-in gaps, whereas any damaged or mismatched bases on one strand of a fully duplexed region which is not resynthesized prior to adapter ligation could be resolved computationally with duplex sequencing if left uncorrected. Further, when these resultant duplexes (either intact or broken apart (e.g., where strand break occurs) are then exposed (e.g., contacted) to an enzyme capable of digesting 5′ overhangs, any 5′ overhangs would be substantially reduced in length, limiting their subsequent fill-in in step (b) to the very ends of the fragment. Then, when the resultant duplexes are exposed (e.g., contacted) to a DNA-dependent DNA polymerase lacking both strand displacement and 5′ exonuclease activity but capable of fill-in of single-stranded segments of the sample and digestion of 3′ overhangs, and a polynucleotide kinase, any short remaining 5′ overhangs which had not been fully digested in the prior step would be filled in to achieve a blunt end; any remaining 3′ overhangs would be digested to produce a blunt end; and any interior gaps (e.g., the small gaps produced by excision of damaged bases and cleaving of abasic sites, and longer gaps which may also exist in DNA fragments) would be filled up to the 5′ end of the downstream DNA segment. Next, when the resultant duplexes are exposed (e.g., contacted) to a DNA ligase capable of sealing nicks (preferably with minimal end-joining activity, so as to avoid chimera formation) any remaining nicks (e.g., those left after gap filling, among others inherently present in the sample) will be sealed, forming a continuous, blunted duplex. Then, when the resultant duplexes are exposed (e.g., contacted) to a DNA polymerase capable of performing non-templated extension (e.g., addition) of dAMP to the 3′ ends of the DNA duplex (e.g., dA-tailing), using DNA polymerases such as Taq or Klenow fragment which bear 5′ exonuclease and strand displacement activity, respectively, there will be substantially fewer ‘priming sites’ available for strand resynthesis. Further, if step (d) is performed under conditions which limit the addition of nucleotides other than dAMP (e.g., by substantially removing dNTPs prior to this step, or by providing dATP in extreme excess), the potential for strand resynthesis in this step can be substantially mitigated. This preserved information allows for greater accuracy and resolution of mutations.

The term “contacted,” as may be used herein, is used to describe the exposure of one substance (e.g., enzyme, reagent, dNTP) to another substance (e.g., sample, mixture), in an amount and with the intention that the two substance interact in a way to effectuate activity of one of the substances on, or to interact with, the other (e.g., an enzyme acting upon a sample). The term is not to be construed to require physical contact between the two substances, but further does not prohibit physical contact either. For example, proximity may be sufficient to affect the interaction and/or activity of the substances with one another. In some embodiments, contact is accomplished by introducing the substances into the same container (e.g., reaction vessel). In some embodiments contact is accomplished by introducing the substances into the same reaction vessel. In some embodiments, contact is accomplished by introducing substance A (e.g., reagent, dNTP, enzyme, etc.) into a reaction vessel, which either contains substance B (e.g., sample), to which substance B is simultaneously introduces, or to which substance B is later introduced. In some embodiments, contact is accomplished when substances physically touch one another (e.g., interact physically). In some embodiments, contact is accomplished when substances chemically interact with one another. In some embodiments, contact is accomplished when substances, enzymatically interact with one another. In some embodiments contact is accomplished when substances are proximal to one another.

In some embodiments, the methods of the disclosure further comprise: (d) preparing the sample for adapter ligation, wherein the preparing comprises: (i) adding deoxyadenosine monophosphate (dAMP) to the 3′ ends of the strands of the sample (dA-tailing); or (ii) blunting the ends of the sample. In some embodiments, dA-tailing comprises, contacting a sample with an enzyme capable of incorporating deoxyadenosine monophosphate (dAMP) to the 3′ end of a strand of the sample and contacting the sample with dNTPs. In some embodiments, enzymes and/or dNTPs used in steps (a)-(c) of the methods of the disclosure are substantially removed from the reaction vessel prior to dA-tailing. In some embodiments, dNTPs substantially comprise dATPs. In some embodiments, one or more (e.g., 1, 2, 3, 4, 5, or more, as representative of steps (a), (b), (c), (d), etc.) of the methods as disclosed herein are performed in a “one-pot” reaction wherein the steps are performed through sequential addition of enzymes and buffers to the same reaction vessel and adjusting reaction conditions (e.g., temperature). In some embodiments, steps are performed sequentially. In some embodiments, reagents and enzymes from the prior step are not removed from the mixture prior to proceeding with a subsequent step. In some embodiments, reagents and enzymes from the prior step are removed from the mixture prior to proceeding with a subsequent step. In some embodiments, one or more steps are performed in one reaction vessel. In some embodiments, one or more steps are performed in more than one reaction vessel (e.g., transferred at least at one time-point throughout a method).

In some embodiments, a sample is contacted by the one or more enzymes of step (a) for at least 15 seconds (e.g., 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more seconds) prior to proceeding with any subsequent steps of a method. In some embodiments, a sample is contacted by the one or more enzymes of step (a) for at least 1 minute (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more minutes) prior to proceeding with any subsequent steps of a method. In some embodiments, a sample is contacted by the one or more enzymes of step (a) and incubated for at least 5 minutes (min) prior to proceeding with any subsequent steps of a method. In some embodiments, a sample is contacted by the one or more enzymes of step (a) and incubated for at least 25 minutes (min) prior to proceeding with any subsequent steps of a method. In some embodiments, a sample is contacted by the one or more enzymes of step (a) and incubated for at least 30 minutes (min) prior to proceeding with any subsequent steps of a method. In some embodiments, a sample is contacted by the one or more enzymes of step (a) for less than 6 hours (e.g., 6, 5, 4, 3, 2, 1, or less hours) prior to proceeding with any subsequent steps of a method. In some embodiments, a sample is contacted by the one or more enzymes of step (a) for less than 60 minutes (e.g., 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or less minutes) prior to proceeding with any subsequent steps of a method. In some embodiments, a sample is contacted by the one or more enzymes of step (a) for between 1 and 60 minutes prior to proceeding with any subsequent steps of a method. In some embodiments, a sample is contacted by the one or more enzymes of step (a) for between 10 and 45 minutes prior to proceeding with any subsequent steps of a method. In some embodiments, a sample is contacted by the one or more enzymes of step (a) for between 20 and 35 minutes prior to proceeding with any subsequent steps of a method.

In some embodiments, a sample is contacted by the one or more enzymes of step (b) for at least 15 seconds (e.g., 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more seconds) prior to proceeding with any subsequent steps of a method. In some embodiments, a sample is contacted by the one or more enzymes of step (b) for at least 1 minute (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more minutes) prior to proceeding with any subsequent steps of a method. In some embodiments, a sample is contacted by the one or more enzymes of step (b) and incubated for at least 5 minutes (min) prior to proceeding with any subsequent steps of a method. In some embodiments, a sample is contacted by the one or more enzymes of step (b) and incubated for at least 25 minutes (min) prior to proceeding with any subsequent steps of a method. In some embodiments, a sample is contacted by the one or more enzymes of step (b) and incubated for at least 30 minutes (min) prior to proceeding with any subsequent steps of a method. In some embodiments, a sample is contacted by the one or more enzymes of step (b) for less than 6 hours (e.g., 6, 5, 4, 3, 2, 1, or less hours) prior to proceeding with any subsequent steps of a method. In some embodiments, a sample is contacted by the one or more enzymes of step (b) for less than 60 minutes (e.g., 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or less minutes) prior to proceeding with any subsequent steps of a method. In some embodiments, a sample is contacted by the one or more enzymes of step (b) for between 1 and 60 minutes prior to proceeding with any subsequent steps of a method. In some embodiments, a sample is contacted by the one or more enzymes of step (b) for between 10 and 45 minutes prior to proceeding with any subsequent steps of a method. In some embodiments, a sample is contacted by the one or more enzymes of step (b) for between 20 and 35 minutes prior to proceeding with any subsequent steps of a method.

In some embodiments, a sample is contacted by the one or more enzymes of step (c) for at least 15 seconds (e.g., 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more seconds) prior to proceeding with any subsequent steps of a method. In some embodiments, a sample is contacted by the one or more enzymes of step (c) for at least 1 minute (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more minutes) prior to proceeding with any subsequent steps of a method. In some embodiments, a sample is contacted by the one or more enzymes of step (c) and incubated for at least 5 minutes (min) prior to proceeding with any subsequent steps of a method. In some embodiments, a sample is contacted by the one or more enzymes of step (c) and incubated for at least 25 minutes (min) prior to proceeding with any subsequent steps of a method. In some embodiments, a sample is contacted by the one or more enzymes of step (c) and incubated for at least 30 minutes (min) prior to proceeding with any subsequent steps of a method. In some embodiments, a sample is contacted by the one or more enzymes of step (c) for less than 6 hours (e.g., 6, 5, 4, 3, 2, 1, or less hours) prior to proceeding with any subsequent steps of a method. In some embodiments, a sample is contacted by the one or more enzymes of step (c) for less than 60 minutes (e.g., 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or less minutes) prior to proceeding with any subsequent steps of a method. In some embodiments, a sample is contacted by the one or more enzymes of step (c) for between 1 and 90 minutes prior to proceeding with any subsequent steps of a method. In some embodiments, a sample is contacted by the one or more enzymes of step (c) for between 30 and 60 minutes prior to proceeding with any subsequent steps of a method. In some embodiments, a sample is contacted by the one or more enzymes of step (c) for between 35 and 55 minutes prior to proceeding with any subsequent steps of a method. In some embodiments, where temperature cycling may occur, a contacting time as described herein, may be for exposure to any of the temperatures, or for any of the portion of the cycling of the temperatures of the step to which it pertains.

In some embodiments, a sample is contacted by the one or more enzymes of step (d) for at least 15 seconds (e.g., 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more seconds) prior to proceeding with any subsequent steps of a method. In some embodiments, a sample is contacted by the one or more enzymes of step (d) for at least 1 minute (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more minutes) prior to proceeding with any subsequent steps of a method. In some embodiments, a sample is contacted by the one or more enzymes of step (d) and incubated for at least 5 minutes (min) prior to proceeding with any subsequent steps of a method. In some embodiments, a sample is contacted by the one or more enzymes of step (d) and incubated for at least 25 minutes (min) prior to proceeding with any subsequent steps of a method. In some embodiments, a sample is contacted by the one or more enzymes of step (d) and incubated for at least 30 minutes (min) prior to proceeding with any subsequent steps of a method. In some embodiments, a sample is contacted by the one or more enzymes of step (d) for less than 6 hours (e.g., 6, 5, 4, 3, 2, 1, or less hours) prior to proceeding with any subsequent steps of a method. In some embodiments, a sample is contacted by the one or more enzymes of step (d) for less than 60 minutes (e.g., 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or less minutes) prior to proceeding with any subsequent steps of a method. In some embodiments, a sample is contacted by the one or more enzymes of step (d) for between 1 and 60 minutes prior to proceeding with any subsequent steps of a method. In some embodiments, a sample is contacted by the one or more enzymes of step (d) for between 10 and 45 minutes prior to proceeding with any subsequent steps of a method. In some embodiments, a sample is contacted by the one or more enzymes of step (d) for between 20 and 35 minutes prior to proceeding with any subsequent steps of a method.

In some embodiments, a sample is contacted by the one or more enzymes of step (d) and incubated for a second period of at least 15 seconds (e.g., 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more seconds) prior to proceeding with any subsequent steps of a method. In some embodiments, a second period is at least 1 minute (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more minutes). In some embodiments, a second period is at least 5 minutes (min). In some embodiments, a second period is at least 25 minutes (min). In some embodiments, a second period is at least 30 minutes (min). In some embodiments, a second period is less than 6 hours (e.g., 6, 5, 4, 3, 2, 1, or less hours). In some embodiments, a second period is less than 60 minutes (e.g., 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or less minutes). In some embodiments, a second period is between 1 and 60 minutes. In some embodiments, a second period is between 10 and 45 minutes. In some embodiments, a second period is between 20 and 35 minutes prior to proceeding with any subsequent steps of a method.

In some embodiments, step (a) of any of the methods disclosed herein is carried out at a temperature between about 20° C. to about 50° C. (e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50° C.). In some embodiments, step (a) of any of the methods disclosed herein is carried out at a temperature between about 25° C. to about 45° C. In some embodiments, step (a) of any of the methods disclosed herein is carried out at a temperature between about 30° C. to about 40° C. In some embodiments, step (a) of any of the methods disclosed herein is carried out at a temperature between about 35° C. to about 39° C. In some embodiments, step (a) of any of the methods disclosed herein is carried out at a temperature of about 37° C.

In some embodiments, step (b) of any of the methods disclosed herein is carried out at a temperature between about 20° C. to about 50° C. (e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50° C.). In some embodiments, step (b) of any of the methods disclosed herein is carried out at a temperature between about 25° C. to about 45° C. In some embodiments, step (b) of any of the methods disclosed herein is carried out at a temperature between about 30° C. to about 40° C. In some embodiments, step (b) of any of the methods disclosed herein is carried out at a temperature between about 35° C. to about 39° C. In some embodiments, step (b) of any of the methods disclosed herein is carried out at a temperature of about 37° C.

In some embodiments, the steps of any of the methods disclosed herein, may be performed at multiple temperatures to facilitate the enzymatic reactions. For example, without limitation, when repeated exposure and ‘cycling’ is desired, the use of manual or automated cycling of the temperature may be used. Techniques, methods, and protocols for such cycling is well known in the art. In some embodiments, cycling may be performed on an automatic thermocycler. In some embodiments, cycling may have two temperature set points, a first temperature and a second temperature.

In some embodiments, step (c) of any of the methods disclosed herein is carried out at a first temperature between about 20° C. to about 50° C. (e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50° C.). In some embodiments, step (c) of any of the methods disclosed herein is carried out at a first temperature between about 25° C. to about 45° C. In some embodiments, step (c) of any of the methods disclosed herein is carried out at a first temperature between about 30° C. to about 40° C. In some embodiments, step (c) of any of the methods disclosed herein is carried out at a first temperature between about 33° C. to about 37° C. In some embodiments, step (c) of any of the methods disclosed herein is carried out at a first temperature of about 35° C.

In some embodiments, step (c) of any of the methods disclosed herein is carried out at a second temperature between about 40° C. to about 80° C. (e.g., 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80° C.). In some embodiments, step (c) of any of the methods disclosed herein is carried out at a second temperature between about 55° C. to about 75° C. In some embodiments, step (c) of any of the methods disclosed herein is carried out at a second temperature between about 60° C. to about 70° C. In some embodiments, step (c) of any of the methods disclosed herein is carried out at a second temperature between about 63° C. to about 67° C. In some embodiments, step (c) of any of the methods disclosed herein is carried out at a second temperature of about 65° C.

In some embodiments, step (d) of any of the methods disclosed herein is carried out at a temperature between about 18° C. to about 70° C. In some embodiments, step (d) of any of the methods disclosed herein is carried out at a temperature between about 20° C. to about 66° C. In some embodiments, step (d) of a method as described herein is carried out at two different temperatures, temperature 1 and temperature 2.

In some embodiments, step (d) of any of the methods disclosed herein is carried out at a temperature 1 of between about 17° C. to about 25° C. (e.g., 17, 18, 19, 20, 21, 22, 23, 24, 25° C.). In some embodiments, step (d) of any of the methods disclosed herein is carried out at a temperature 1 of between about 19° C. to about 23° C. In some embodiments, step (d) of any of the methods disclosed herein is carried out at a temperature 1 of between about 20° C. to about 22° C. In some embodiments, step (d) of any of the methods disclosed herein is carried out at a temperature 1 of about 22° C.

In some embodiments, step (d) of any of the methods disclosed herein is carried out at a temperature 2 of between about 60° C. to about 70° C. (e.g., 60, 61, 62, 63, 64, 65, 66, 67, 68, 69, 70° C.). In some embodiments, step (d) of any of the methods disclosed herein is carried out at a temperature 2 of between about 62° C. to about 68° C. In some embodiments, step (d) of any of the methods disclosed herein is carried out at a temperature 2 of between about 64° C. to about 66° C. In some embodiments, step (d) of any of the methods disclosed herein is carried out at a temperature 2 of about 65° C.

In some embodiments, prior to step (a) a sample has been: (i) fragmented; or (ii) cleaved and tagged (tagmented). In some embodiments, fragmentation is by: (a) physical fragmentation; (b) enzymatic fragmentation; and/or (c) chemical fragmentation. In some embodiments, fragmentation is by physical fragmentation. In some embodiment, physical fragmentation is by nebulization. In some embodiments, physical fragmentation is by acoustic shearing. In some embodiments, physical fragmentation is by needle shearing. In some embodiments, physical fragmentation is by French pressure cell. In some embodiments, physical fragmentation is by sonication. In some embodiments, physical fragmentation is by hydrodynamic shearing. In some embodiments, fragmentation is by enzymatic fragmentation. In some embodiments, enzymatic fragmentation is by nuclease or endonuclease. In some embodiments, enzymatic fragmentation is by DNase I. In some embodiments, enzymatic fragmentation is by restriction endonuclease. In some embodiments, enzymatic fragmentation is by transposase. In some embodiments, is by chemical fragmentation. In some embodiments, chemical fragmentation is by heat and divalent metal cation fragmentation.

In some embodiments, step (a) comprises contacting the sample with one or more enzymes selected from the group consisting of: (1) endonuclease IV (EndoIV); (2) formamidopyrimidine [fapy]-DNA glycosylase (Fpg); (3) uracil-DNA glycosylase (UDG); (4) T4 pyrimidine DNA glycosylase (T4 PDG); (5) endonuclease VIII (EndoVIII), and (6) exonuclease VII (ExoVII).

The term “glycosylase,” as may be used herein, refers to the term of art generally known to the skilled artisan to refer to an enzyme which is primarily involved with the repair of nucleic acids (e.g., DNA). The primary activity by which glycosylases aid in the repair of DNA is by base excision repair, which removes damaged DNA and replaces it with new, fresh DNA without errors (e.g., removes or repairs damaged bases (e.g., lesions)). Glycosylases interact with the damaged nitrogenous section of the DNA while leaving the backbone (e.g., sugar-phosphate group) intact. This excision allows for the synthesis and replacement of the damaged base (e.g., insertion of new DNA) at the site. For example, without limitation, DNA glycosylases excise uracil residuals from DNA by cutting the N-glycosidic bond, which begins the DNA excision repair process. In some embodiments, a glycosylase is selected from: formamidopyrimidine [fapy]-DNA glycosylase (Fpg); glycosylase is uracil-DNA glycosylase (UDG); T4 pyrimidine DNA glycosylase (T4 PDG); or a combination thereof. In some embodiments, a glycosylase is formamidopyrimidine [fapy]-DNA glycosylase (Fpg). In some embodiments, a glycosylase is uracil-DNA glycosylase (UDG). In some embodiments, a glycosylase is T4 pyrimidine DNA glycosylase (T4 PDG).

In some embodiments, the activity of the one or more enzymes catalyze the following DNA modifications on the sample: (1) excision of damaged bases; and (2) excision of abasic sites. In some embodiments, activity of the one or more enzymes is sequential or simultaneous.

In some embodiments, a damaged bases are selected from the group consisting of: uracil; 8′oxoG; an oxidized pyrimidine; and a cyclobutane pyrimidine dimer.

In some embodiments, a 5′ overhang of at least one strand of the sample is at least 10 nucleobases in length. In some embodiments, a 5′ overhang of at least one strand of the sample is at least 75 nucleobases in length. In some embodiments, a 3′ overhang of at least one strand of the sample is at least 10 nucleobases in length. In some embodiments, a 3′ overhang of at least one strand of the sample is at least 75 nucleobases in length.

In some embodiments, one or more enzymes digests a 5′ overhang of at least one strand of the sample to less than 16 nucleobases in length. In some embodiments, one or more enzymes digests a 5′ overhang of at least one strand of the sample to less than 8 nucleobases in length. In some embodiments, one or more enzymes digests a 3′ overhang of at least one strand of the sample to less than 16 nucleobases in length. In some embodiments, one or more enzymes digests a 3′ overhang of at least one strand of the sample to less than 8 nucleobases in length.

In some embodiments, endonuclease IV (EndoIV) cleaves abasic sites. In some embodiments, formamidopyrimidine [fapy]-DNA glycosylase excises damaged purines. In some embodiments, uracil-DNA glycosylase (UDG) excises uracil. In some embodiments, T4 pyrimidine DNA glycosylase (T4 PDG) excises cyclobutene pyrimidine dimers. In some embodiments, endonuclease VIII (EndoVIII) excises damaged pyrimidines. In some embodiments, DNA ligase is a HiFi Taq DNA ligase.

In some embodiments, step (b) of the methods of the disclosure comprises contacting the DNA fragment with a polynucleotide kinase (Pnk). In some embodiments, a Pnk is a T4 polynucleotide kinase.

In some embodiments of any of the methods of the disclosure: (a) an endonuclease IV (EndoIV) comprises an amino acid sequence with at least 70% identity (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to SEQ ID NO: 3 or any known endonuclease IV sequence; (b) a formamidopyrimidine [fapy]-DNA glycosylase (Fpg) comprises an amino acid sequence with at least 70% identity (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to SEQ ID NO: 4 or any known formamidopyrimidine [fapy]-DNA glycosylase sequence; (c) an uracil-DNA glycosylase (UDG) comprises an amino acid sequence with at least 70% identity (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to an amino acid sequence selected from the group consisting of: SEQ ID NO: 5-7 or any known uracil-DNA glycosylase sequence; (d) a T4 pyrimidine DNA glycosylase (T4 PDG) comprises an amino acid sequence with at least 70% identity (e.g., at least 70%, at least 71%, at least 72%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to any known T4 pyrimidine DNA glycosylase sequence; and/or (e) an endonuclease VIII (EndoVIII) comprises an amino acid sequence with at least 70% identity (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to an amino acid sequence selected from the group consisting of: SEQ ID NO: 8-9 or any known endonuclease VIII sequence.

In some embodiments of any of the methods of the disclosure, a polynucleotide kinase comprises an amino acid sequence with at least 70% identity (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to an amino acid sequence of SEQ ID NO: 10 or any known polynucleotide kinase sequence.

In some embodiments of any of the methods of the disclosure: (1) a DNA-dependent DNA polymerase comprises an amino acid sequence with at least 70% identity (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to any known DNA-dependent DNA polymerase sequence; and/or (2) a DNA ligase comprises an amino acid sequence with at least 70% identity (e.g., at least 70%, at least 71%, at least 72%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) to any known DNA ligase sequence.

In some aspects, the disclosure relates to a method of duplex sequencing that mitigates false mutation detection, comprising: (A1) obtaining a nucleic acid to be sequenced; (A2) performing the method of embodiment 1 or any one of embodiments 2-51; (A3) duplex sequencing the sample; and (A4) identifying mutations by computational analysis.

In some aspects, the disclosure relates to a method of reducing artifact in duplex sequencing, comprising: (A1) obtaining a nucleic acid to be sequenced; (A2) performing the method of embodiment 1 or any one of embodiments 2-51; and (A3) duplex sequencing the sample.

In some aspects, the disclosure relates to a method of reducing synthetic strand synthesis during nucleic acid sample preparation for sequencing, comprising: (A1) obtaining a nucleic acid to be sequenced; and (A2) performing the method of embodiment 1 or any one of embodiments 2-51.

In some aspects, the disclosure relates to a method of increasing the accuracy of mutation identification, comprising: (A1) obtaining a nucleic acid to be sequenced; (A2) performing the method of embodiment 1 or any one of embodiments 2-51; (A3) duplex sequencing the sample; and (A4) identifying mutations by computational analysis.

In some embodiments, a sample is sequenced. In some embodiments, sequencing is sanger-based sequencing. In some embodiments, sequencing is based on high-throughput sequencing (e.g., next generation sequencing). Next generation sequencing, or “NGS,” is well-known in the art and will be readily apparent to the skilled artisan. For example, without limitation, NGS sequencing technologies include those from Life Technologies™ and Illumina™, PacBio, and Oxford Nanopore. In some embodiments, sequencing is duplex sequencing. In some embodiments, the sequencing comprises computational analysis on a computer. In some embodiments, this computational analysis comprises trimming of the sample sequences. Trimming may comprise trimming the sequencing of a given fragment at least one end of a strand. This trimming is performed, at least in part, often to compensate or reduce any errors from false mutations or mismatches that may occur at the ends of a fragment due to strand resynthesis as described elsewhere herein. In some embodiments, trimming occurs at least one end. In some embodiments, trimming occurs at both ends. In some embodiments, at least one nucleotide of the sequence is trimmed (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more). In some embodiments, at least 10 nucleotides are trimmed. In some embodiments, at least 12 nucleotides are trimmed. In some embodiments, less than 30 nucleotides of the sequence are trimmed (e.g., 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1). In some embodiments, less than 15 nucleotides are trimmed. In some embodiments, at least 13 nucleotides are trimmed.

In some aspects, the disclosure relates to a kit comprising: (a) reagents to perform any of the methods of the disclosure; and (b) a container. In some embodiments, a kit further comprises a reaction vessel. In some embodiments, reagents of the kit comprise: (a) one or more of: endonuclease IV (EndoIV); formamidopyrimidine [fapy]-DNA glycosylase (Fpg); uracil-DNA glycosylase (UDG); T4 pyrimidine DNA glycosylase (T4 PDG); and/or endonuclease VIII (EndoVIII); and/or (b) dNTPs. In some embodiments, a kit further comprises reagents and materials to fragment the sample.

The computational analysis can be any suitable algorithm, for example the algorithm described in Parsons et al. Clinical Cancer Research, DOI: 10.1158/1078-0432.CCR-19-3005 Published June 2020, vol. 26, No. 11, pp. 2556-2564, which is incorporated herein by reference in its entirety.

Samples

In some embodiments, a sample as used in any of the methods of the disclosure comprises DNA, RNA, or a combination thereof. In some embodiments, a sample comprises DNA. In some embodiments, a sample comprises RNA. Selection of appropriate samples, and performance of the methods of the present disclosure will be readily apparent to the skilled artisan and will not entail undue experimentation. For example, without limitation, a sample may comprise cell-free DNA (cfDNA) and/or germline DNA. In some embodiments, a sample comprises cfDNA. In some embodiments, a sample comprises germline DNA.

Furthermore, as will be readily apparent, samples may be generated from a variety of sources. The nucleic acids comprising the sample may come from any component of a subject. For example, without limitation, a sample may be blood, saliva, or other cellular component comprising a subject. In some embodiments, the sample is generated from the subject by means of a biopsy. In some embodiments, the biopsy is a liquid biopsy. In some embodiments, the biopsy is a tumor biopsy.

In some embodiments, a sample contains zero gaps (e.g., 0). In some embodiments, a sample comprises at least one gap (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more gaps). In some embodiments, a sample comprises more than one gap (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more gaps). In some embodiments, a sample comprises less than or equal to 10 gaps (e.g., 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 gaps). In some embodiments, a sample comprises less than or equal to 10 gaps (e.g., 10, 9, 8, 7, 6, 5, 4, 3, 2, 1, or 0 gaps). In some embodiments, a sample comprises between 0 and 101 gaps. In some embodiments, a sample comprises between 0 and 11 gaps. In some embodiments, a sample comprises between 1 and 101 gaps. In some embodiments, a sample comprises between 1 and 11 gaps.

In some embodiments, a gap comprises a single-stranded region of the sample wherein at least one (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more) nucleoside is absent opposite a single-stranded portion of the sample. In some embodiments, a gap comprises a single-stranded region of the sample wherein more than one (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more) nucleosides are absent opposite a single-stranded portion of the duplex. In some embodiments, a gap comprises a single-stranded region of the sample wherein less than 100 (e.g., 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1) nucleosides are absent opposite a single-stranded region of the sample. In some embodiments, a gap comprises as single-stranded region of the sample wherein less than 10 (e.g., 10, 9, 8, 7, 6, 5, 4, 3, 2, or 1) nucleosides are absent opposite a single-stranded region of the sample. In some embodiments, a gap comprises a single-stranded region wherein between 1 and 101 nucleosides are absent opposite a single-stranded region of the sample. In some embodiments, a gap comprises a single-stranded region wherein between 1 and 11 nucleosides are absent opposite a single-stranded region of the sample.

In some embodiments, a sample comprises at least one gap in at least one strand of a sample. In some embodiments a sample comprises at least one gap in both strands of a sample. In some embodiments, a sample comprises more than one gap in at least one strand of a sample. In some embodiments a sample comprises more than one gap in both strands of a sample.

In some embodiments, a sample does not comprise an overhang. In some embodiments, a sample comprises an overhang. In some embodiments, an overhang is at least one nucleoside (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, or more nucleosides) in length. In some embodiments, an overhang is more than one nucleoside in length. In some embodiments, an overhang is less than the length of the sample less the overhang (e.g., less than 50% of the overall length of the sample) in length. In some embodiments, an overhang is less than 350 nucleosides in length (e.g., 350, 349, 348, 347, 346, 345, 344, 343, 342, 341, 340, 339, 338, 337, 336, 335, 334, 333, 332, 331, 330, 329, 328, 327, 326, 325, 324, 323, 322, 321, 320, 319, 318, 317, 316, 315, 314, 313, 312, 311, 310, 309, 308, 307, 306, 305, 304, 303, 302, 301, 300, 299, 298, 297, 296, 295, 294, 293, 292, 291, 290, 289, 288, 287, 286, 285, 284, 283, 282, 281, 280, 279, 278, 277, 276, 275, 274, 273, 272, 271, 270, 269, 268, 267, 266, 265, 264, 263, 262, 261, 260, 259, 258, 257, 256, 255, 254, 253, 252, 251, 250, 249, 248, 247, 246, 245, 244, 243, 242, 241, 240, 239, 238, 237, 236, 235, 234, 233, 232, 231, 230, 229, 228, 227, 226, 225, 224, 223, 222, 221, 220, 219, 218, 217, 216, 215, 214, 213, 212, 211, 210, 209, 208, 207, 206, 205, 204, 203, 202, 201, 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 149, 148, 147, 146, 145, 144, 143, 142, 141, 140, 139, 138, 137, 136, 135, 134, 133, 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). In some embodiments, an overhang is less than 100 nucleosides in length. In some embodiments, an overhang is between 0 and 100 nucleosides in length. In some embodiments, an overhang is between 1 and 350 nucleosides in length. In some embodiments, an overhang is between 1 and 100 nucleosides in length. In some embodiments, an overhang is between 1 and 50 nucleosides in length.

In some embodiments, a sample comprises no overhangs. In some embodiments, a sample comprises at least one (e.g., 1, 2) overhang. In some embodiments, a sample comprises two overhangs. In some embodiments, a sample comprises at least one 5′ overhang. In some embodiments, a sample comprises two 5′ overhangs. In some embodiments, a sample comprises at least one 3′ overhang. In some embodiments, a sample comprises two 3′ overhangs. In some embodiments, a sample comprises a 5′ overhang and a 3′ overhang.

In some embodiments, a sample contains zero nicks (e.g., 0). In some embodiments, a sample comprises at least one nick (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more nicks). In some embodiments, a sample comprises more than one nick (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more nicks). In some embodiments, a sample comprises less than or equal to 10 nicks (e.g., 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 nicks). In some embodiments, a sample comprises less than or equal to 10 nicks (e.g., 10, 9, 8, 7, 6, 5, 4, 3, 2, 1, or 0 nicks). In some embodiments, a sample comprises between 0 and 101 nicks. In some embodiments, a sample comprises between 0 and 11 nicks. In some embodiments, a sample comprises between 1 and 101 nicks. In some embodiments, a sample comprises between 1 and 11 nicks.

In some embodiments, a sample comprises at least one nick in at least one strand of a sample. In some embodiments a sample comprises at least one nick in both strands of a sample. In some embodiments, a sample comprises more than one nick in at least one strand of a sample. In some embodiments a sample comprises more than one nick in both strands of a sample.

In some embodiments, a sample contains zero damaged bases (e.g., 0). In some embodiments, a sample comprises at least one damaged base (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more damaged bases). In some embodiments, a sample comprises more than one damaged base (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more damaged bases). In some embodiments, a sample comprises less than or equal to 10 damaged bases (e.g., 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 damaged bases). In some embodiments, a sample comprises less than or equal to 10 damaged bases (e.g., 10, 9, 8, 7, 6, 5, 4, 3, 2, 1, or 0 damaged bases). In some embodiments, a sample comprises between 0 and 101 damaged bases. In some embodiments, a sample comprises between 0 and 11 damaged bases. In some embodiments, a sample comprises between 1 and 101 damaged bases. In some embodiments, a sample comprises between 1 and 11 damaged bases.

In some embodiments, a sample comprises at least one damaged base in at least one strand of a sample. In some embodiments a sample comprises at least one damaged base in both strands of a sample. In some embodiments, a sample comprises more than one damaged base in at least one strand. In some embodiments, a sample comprises a damaged base in a double-stranded portion of the sample. In some embodiments, a sample comprises a damaged base in a single-stranded portion of the sample. In some embodiments, a sample comprises a damaged base in both a single-stranded and a double-stranded portion of the sample.

In some embodiments, a sample contains zero mismatches (e.g., 0). In some embodiments, a sample comprises at least one mismatch (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more mismatches). In some embodiments, a sample comprises more than one mismatch (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more mismatches). In some embodiments, a sample comprises less than or equal to 10 mismatches (e.g., 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 mismatches). In some embodiments, a sample comprises less than or equal to 10 mismatches (e.g., 10, 9, 8, 7, 6, 5, 4, 3, 2, 1, or 0 mismatches). In some embodiments, a sample comprises between 0 and 101 mismatches. In some embodiments, a sample comprises between 0 and 11 mismatches. In some embodiments, a sample comprises between 1 and 101 mismatches. In some embodiments, a sample comprises between 1 and 11 mismatches.

The terms “percent identity,” “sequence identity,” “% identity,” “% sequence identity,” and % identical,” as they may be interchangeably used herein, refer to a quantitative measurement of the similarity between two sequences (e.g., nucleic acid or amino acid). The percent identity of genomic DNA sequence, intron and exon sequence, and amino acid sequence between humans and other species varies by species type, with chimpanzee having the highest percent identity with humans of all species in each category.

Calculation of the percent identity of two nucleic acid sequences, for example, can be performed by aligning the two sequences for optimal comparison purposes (e.g., gaps can be introduced in one or both of a first and second nucleic acid sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Atschul, S. F. et al., J. Molec. Biol., 215, 403 (1990)).

When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (e.g., 0.1%), hundredths of a percent (e.g., 0.01%), etc.).

Unless otherwise defined herein, scientific and technical terms used in connection with the present disclosure shall have the meanings that are commonly understood by those of ordinary skill in the art (e.g., the skilled artisan). The meaning and scope of the terms are clear; however, in the event of any latent ambiguity, definitions provided herein take precedent over any dictionary or extrinsic definition. Further, unless otherwise required by context, singular terms shall include pluralities and plural terms shall include the singular. In this disclosure, the use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics, and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present disclosure unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of subjects.

The terms “approximately” or “about,” as may be used interchangeably herein, and as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction of (i.e., percentage greater than or percentage less than) the stated reference value unless otherwise stated or otherwise evident from the context (for example, when such number would exceed 100% of a possible value).

### EXAMPLES

### Example 1: Duplex-Repair Limits False Mutation Discovery in Duplex Sequencing

As more tests based on next generation sequencing (NGS) are advancing toward clinical use, it is imperative to maximize NGS accuracy. This is particularly important when seeking to detect low-abundance mutations in clinical specimens, such as for early cancer detection (Chabon et al., Nature, 2020; Corcoran et al., Ann Rev Cancer Bio, 2019), monitoring of minimal residual disease (“MRD”) (Parsons et al., Clinic Cancer Res, 2020; Tie et al., Sci Trans Med, 2016), tracing of actionable or resistance mutations (Parikh et al., Nat Med, 2019), performing prenatal genetic test (Lo et al., Sci Trans Med, 2010) and detecting microbial or viral infections (Blauwkamp et al., 2019), as errors could lead to incorrect diagnoses and treatments. In addition, high accuracy NGS is desired in research applications such as for studying somatic mosaicism (Dou et al., Trends in Genetics, 2018) and clonal hematopoiesis (Genovese et al., 2014), evaluating the mutagenicity of chemical compounds (Matsumura et al., 2018), characterizing base editing technologies such as clustered regularly interspaced short palindromic repeats (“CRISPR”) (Anzalone, 2020), and using DNA to store digital data (Ceze et al., Nat Rev Genetics, 2019), as errors could lead to unfounded biological discoveries or incorrect (de)coding of information.

DNA base damage is a major source of false mutation discovery in NGS (Chen et al., Science, 2017). Lesions such as cytosine deamination, thymine dimers, pyrimidine dimers, 8-Oxoguanine, 6-O-methylguanine, depurination, and depyrimidination arise both spontaneously and in response to environmental and chemical exposures such as ultraviolet (UV) radiation, ionization radiation, reactive oxygen species, and genotoxic agents, or sample processing procedures, such as formalin fixation, freezing and thawing, heating, acoustic shearing, and long-term storage in aqueous solution (Costello et al., Nucleic Acids Res, 2013; Wong et al., BMC Med Genomics, 2014). If left uncorrected, such lesions could result in altered base pairing when copied by a polymerase capable of translesion synthesis, thereby leading to detection of a false mutation. These problems, along with other errors introduced in library amplification and sequencing, contribute to an error rate of 0.1%-1% in standard NGS (Salk et al., Nat Rev Genetics, 2018).

Due to the stochasticity of base damage errors, many can be overcome by sequencing multiple copies of each DNA fragment and requiring a consensus among reads. Such “consensus-based” sequencing can reduce errors by up to 100-fold, when requiring a consensus from each single strand of DNA, and up to 1000-fold, when requiring a consensus from both sense strands of each DNA duplex. Methods requiring the sequencing and reading of both sense strands of a duplex are known as “duplex sequencing” (Schmitt et al., PNAS, 2012). However, existing methods for ‘end repair/dA-tailing’ (ER/AT) which are used to correct backbone damages (e.g., nicks, gaps, and overhangs) in duplex DNA, and facilitate ligation of NGS adapters, could resynthesize portions of each duplex prior to adapter ligation. If resynthesis occurs in the presence of base damage, translesion synthesis could copy errors to both strands and render them indistinguishable from true mutations on both strands. This major source of false discovery in duplex sequencing is most clearly seen at fragment ends where short 5′ overhangs are often filled in. Yet, this could also span much deeper given (i) the 5′ exonuclease and strand-displacement activities of Taq and Klenow polymerases used in ER/AT, and (ii) the varied backbone damages that could act as ‘priming sites’ for strand resynthesis.

Presented herein is an approach called Duplex-Repair to limit the potential for base damage errors to be copied to both strands (FIG. 1), by at least limiting strand resynthesis. Using both single-molecule real-time and gene panel sequencing of cfDNA and FFPE tumor biopsies, it is shown that Duplex-Repair minimizes strand resynthesis and limits false mutation discovery in duplex sequencing.

### Commercial End Repair/dA-Tailing (ER/AT) Kits Perform Extensive DNA Resynthesis

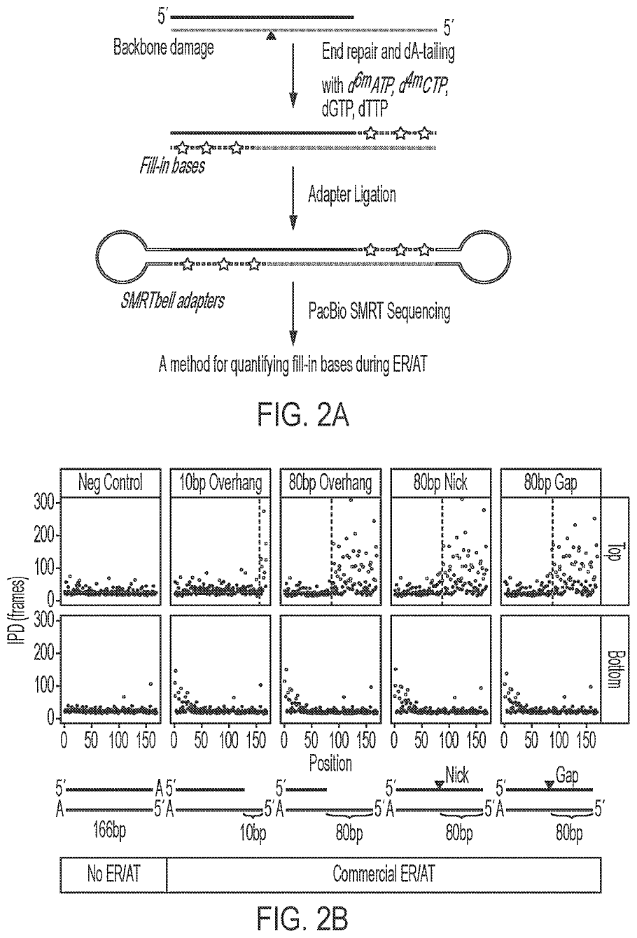

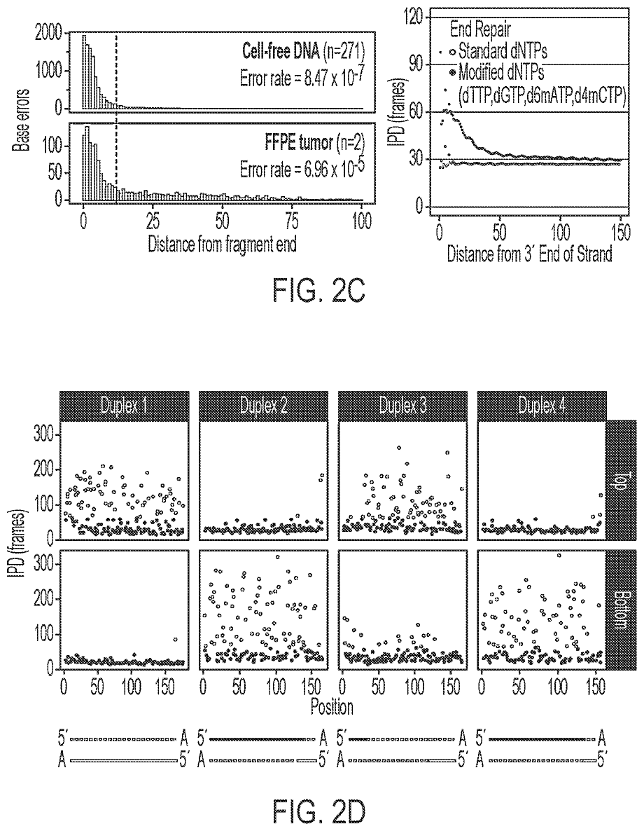

First, an assay was developed to measure the number of bases resynthesized by ER/AT methods. This technique involved performing ER/AT using a custom dNTP mix consisting of d6mATP, d4mCTP, dTTP, and dGTP, and sequencing the prepared libraries on a PacBio sequencer that can detect where d6mATP and d4mCTP have been incorporated (FIG. 2A). To confirm the assay could reliably detect resynthesized bases with single nucleotide resolution, three synthetic oligonucleotides were prepared: a perfect duplex (including adenosine overhangs for dA-tailed ligation of NGS adapters); an oligonucleotide with a 10 base pair 5′ overhang; and an oligonucleotide with an 80 base pair 5′ overhang. As expected, fill-in was observed in the overhang regions of the top strands, confirming that commercial ER/AT fills in 5′ overhangs. Furthermore, fill-in bases were detected upstream of the 3′ termini on both top and bottom strands, suggesting that polymerases, such as T4 DNA polymerase could chew back (e.g., degrade) 3′ termini before initiating fill-in, which further increased the extent of DNA polymerization (FIG. 2B). Next, testing was performed to determine whether commercial ER/AT kits resynthesized all bases downstream from a nick or gap site. A short oligo was annealed to the synthetic oligonucleotide with an 80 base pair 5′ overhang to form a full duplex with one artificial nick or 1 nucleotide gap at the same location and showed that the entire region downstream of the nick or gap site was filled when subjected to commercial ER/AT (FIG. 2B). Resynthesis was also detected upstream of the 3′ termini on both top and bottom strands.

This technique was applied to cfDNA from five healthy donors. The fill-in occurred predominantly near 3′ ends although it could extend much deeper into the fragments (FIG. 2C). In some cases, the majority of one strand or an entire strand was resynthesized during ER/AT by using a commercial kit (FIG. 2D). Overall, the results suggest that commercial ER/AT kits perform extensive DNA resynthesis while attempting to correct backbone damage in cfDNA, meaning that most base pairs sequenced might not be from the original cfDNA duplexes.

Strand Resynthesis is Most Problematic where there is Base Damage

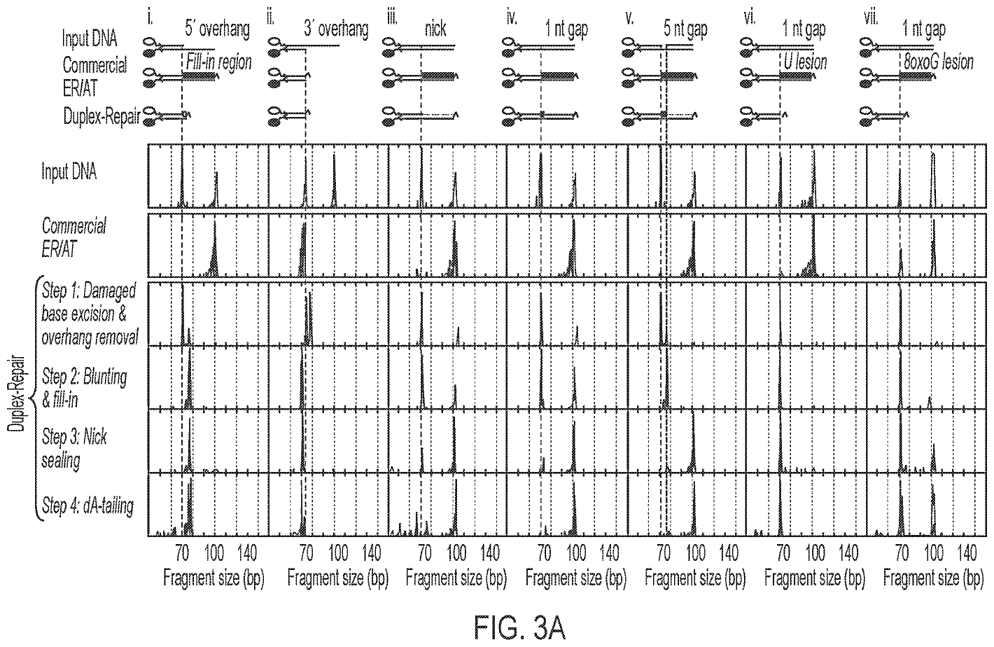

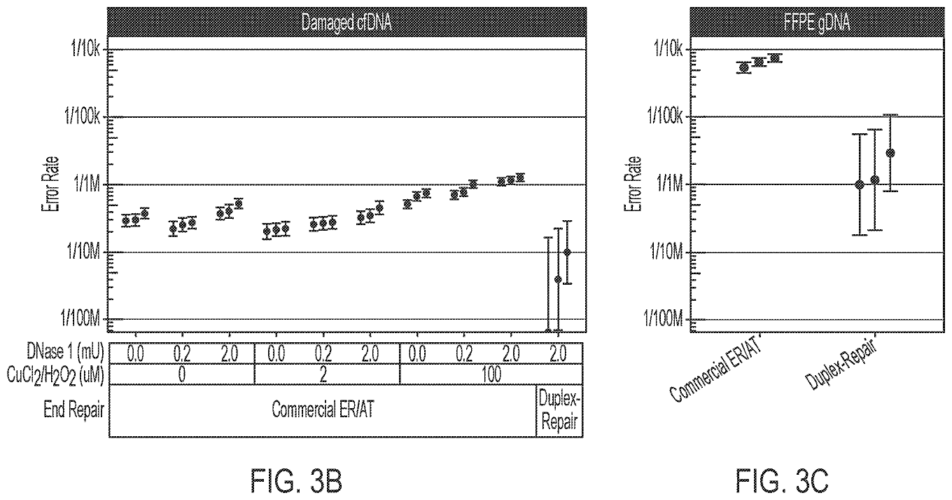

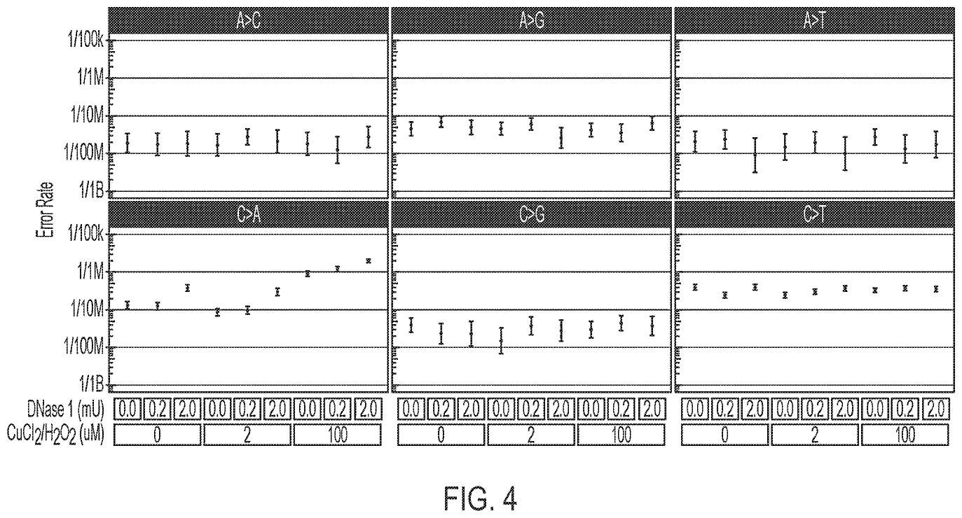

Cell-free DNA (cfDNA) from one healthy donor was subjected to different concentrations of DNase I (to induce further nicks) and the oxidizing agent CuCl₂/H₂O₂. Targeted duplex sequencing of the IDT xGen pan-cancer gene panel was then applied to each sample and detected the highest error rate when cfDNA was treated with the highest concentrations of DNase I and CuCl₂/H₂O₂ (FIG. 3B). At the same concentration of CuCl₂/H₂O₂, the measured error rate increased with the amount of DNase I used. This confirmed that a higher extent of strand resynthesis from nicks induced by a higher amount of DNase I could propagate more base damage errors and increase duplex sequencing error rates. The highest error rates were observed in duplex sequencing with the maximum concentrations of DNase I and CuCl₂/H₂O₂. Also, it was confirmed that mutations observed matched the expected mutation signature of CuCl₂/H₂O₂ exposure (FIG. 4).

Duplex-Repair Limits DNA Polymerization During DNA End Repair and dA-Tailing

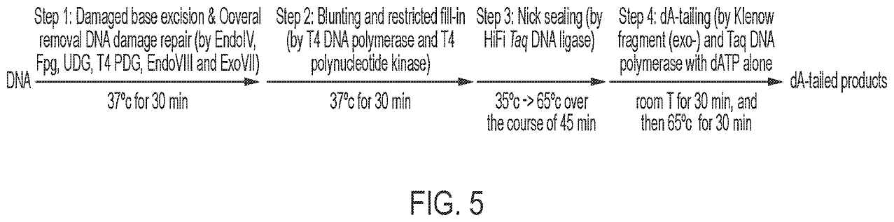

Duplex-Repair is a custom method/kit to limit errors introduced by existing ER/AT methods prior to adapter ligation (FIG. 1, FIG. 5). Duplex-Repair consists of four steps: (1) damaged base excision and overhang removal, (2) blunting and restricted fill-in, (3) nick sealing, and (4) dA-tailing. In step 1, DNA is treated with an enzyme cocktail consisting of Endonuclease IV (EndoIV), Formamidopyrimidine [fapy]-DNA glycosylase (Fpg), Uracil-DNA glycosylase (UDG), T4 pyrimidine DNA glycosylase (T4 PDG) and Endonuclease VIII (EndoVIII). The simultaneous activity of these enzymes excises damaged bases such as Uracil, 8′oxoG, oxidized pyrimidines, cyclobutane pyrimidine dimers and abasic sites, resulting in 1 nucleotide gaps in double-stranded regions or strand breaks in single-strand regions. Exonuclease VII (ExoVII) is employed in this step to degrade 3′ and 5′ single-strand overhangs. In step 2, T4 polynucleotide kinase (de)phosphorylates DNA termini and T4 DNA polymerase (with 3′->5′ exonuclease activity but no 5′ to 3′ exonuclease or strand displacement activity) blunts 3′ overhangs and fills in gaps and the short (≤7 nt) remaining 5′ overhangs. Nicks are sealed by HiFi Taq DNA ligase, selected to minimize spurious intermolecular ligation, in step 3. dA-tailing in step 4 is performed by employing Klenow fragment (exo-) and Taq DNA polymerase with only dATP present to prevent DNA resynthesis. To verify the performance of Duplex-Repair relative to commercial ER/AT, synthetic oligonucleotides labelled with fluorophores and containing multiple types of backbone and base damages were analyzed by capillary electrophoresis (FIG. 3A).

Synthetic oligos with 5′ overhangs: A dsDNA substrate was prepared with a 30-base pair 5′ overhang and two different nuclease-resistant fluorophores at the other terminus (FIG. 3A, Column i). With a commercial ER/AT kit, 101 base pair dA-tailed products were detected, suggesting that DNA polymerases resynthesized the 30 base pairs complementary to the entire 5′ overhang. In contrast, with Duplex-Repair, the 30 base pair 5′ overhang was degraded to 3 base pair after step 1 and only 3 nucleotides were filled in during step 2, shown by the 73 base pair dA tailed products.

Synthetic oligos with 3′ overhangs: A dsDNA substrate was prepared with a 30 base pair 3′ overhang and observed that the commercial kit yielded 71 base pair dA-tailed products, suggesting that the 3′ overhang was fully blunted and there was no fill-in (FIG. 3A, Column ii). Similarly, with Duplex-Repair, the 3′ overhang was blunted after the first two steps, as the dA-tailed products are also 71 bp.

Synthetic oligos with nicks: A 30 base pair oligo was annealed to the 30 base pair 5′ overhang substrate to make dsDNA with an artificial nick and detected 101 base pair dA-tailed products with the commercial ER/AT kit, suggesting that DNA polymerases filled in 30 nucleotide by nick translation or strand displacement to make a 101 base pair top strand product (as there was no DNA ligase to seal the nick, FIG. 3A, Column iii). With Duplex-Repair, in step 2, T4 DNA polymerase did not extend the top strand from the nick site due to its lack of nick translating or strand displacing activity, and the nick was efficiently sealed by HiFi Taq DNA ligase in step 3.

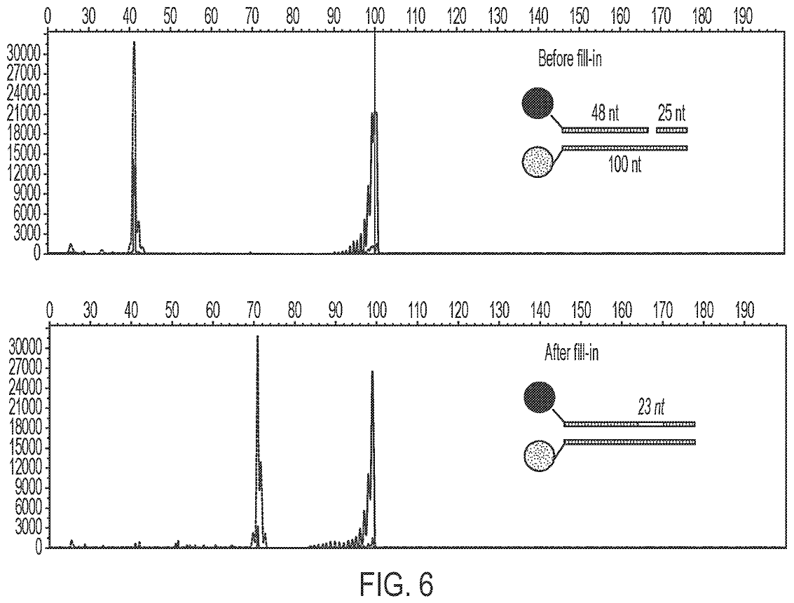

Synthetic oligos without a base damage in gap regions: A 29 base pair or 25 base pair oligo was annealed to the dsDNA with a 30 base pair 5′ overhang to make a dsDNA with a 1 or 5 nucleotide gap and observed that DNA polymerases in the commercial kit copied through the bottom strand from the gap site by nick translation or strand displacement, filling in 30 nucleotide and generating 101 base pair dA-tailed products (FIG. 3A, Columns iv and v). However, with Duplex-Repair, during step 2, T4 DNA polymerase efficiently filled in the 1 nucleotide or 5 nucleotide gap without further resynthesis (it was also observed that T4 DNA polymerase could efficiently fill in a 27 nucleotide gap (FIG. 6)), and the resulting nicks were efficiently sealed by HiFi Taq DNA ligase during step 3.

Synthetic oligos with a base damage in gap regions: A 29 base pair oligo was annealed to the dsDNA with a 30 base pair 5′ overhang to make a dsDNA with a 1 nucleotide gap and a Uracil or 8′oxoG lesion opposing the gap region (FIG. 3A, Columns vi and vii). 101 base pair dA-tailed products were detected with the commercial kit, again suggesting that DNA polymerases copied the bottom strand that contains a base damage which would propagate the base damage error onto both strands. In contrast, with Duplex-Repair, 70 base pair products were detected after step 1, suggesting that the intended strand break occurred at the base damage position, thus preventing base damage errors from being copied onto both strands. Step 4 yielded 71 base pair dA-tailed products.

### Duplex-Repair Limits Duplex Sequencing Errors in Real Clinical Samples

To test if Duplex-Repair can limit duplex sequencing errors, ER/AT was performed on the most heavily damaged cfDNA from FIG. 3B and a FFPE gDNA sample using Duplex-Repair v. the commercial kit, and then applied targeted sequencing of the IDT xGen pan-cancer panel or a custom panel. It was observed that Duplex-Repair exhibited 20-fold and 60-fold error reduction for the damaged cfDNA sample, (1×10⁻⁶ to 5×10⁻⁸) and FFPE gDNA sample (6×10⁻⁵ to 1×⁻⁶), respectively, relative to the commercial ER/AT kit (FIG. 3C). The error rates of the most heavily damaged cfDNA repaired by Duplex-Repair are even lower than the undamaged cfDNA prepared using commercial ER/AT. The results confirm that Duplex-Repair can protect against the propagation of base damage errors in samples and improve the fidelity of duplex sequencing.

### Example 2: Duplex-Repair Enables Highly Accurate Sequencing Despite DNA Damage

DNA sequences of synthetic Mutations in DNA drive genetic diversity¹, alter gene function², impact cellular phenotypes³, mark cell populations⁴, define evolutionary trajectories⁵, underscore diseases and conditions⁶, and provide targets for precision medicines and diagnostics. It is thus crucial to be able to detect mutations across a wide range of abundances. For instance, detecting low-abundance mutations (e.g. <0.1-1% VAF, down to ‘single duplex’ resolution) is important for studying cancer evolution⁸ and drug resistance⁹, understanding somatic mosaicism¹⁰ and clonal hematopoiesis¹¹, characterizing base editing technologies¹², evaluating the mutagenicity of chemical compounds¹³, uncovering pathogenic variants¹⁴, studying human embryonic development¹⁵, detecting microbial or viral infections¹⁶ and cancers¹⁷ and clinically actionable genomic alterations from specimens such as tissue or liquid biopsies¹⁸, and much more.

Despite progress in next generation sequencing (NGS), DNA damage confounds mutation detection and renders accuracy dependent upon sample quality, which is deeply problematic¹⁹. Lesions such as uracil, thymine dimers, pyrimidine dimers, 8-oxoGuanine (8′oxoG), 6-O-methylguanine, depurination, and depyrimidination arise both spontaneously and in response to environmental and chemical exposures, such as UV radiation, ionization radiation, reactive oxygen species, and genotoxic agents, or sample processing procedures, such as formalin fixation, freezing and thawing, heating and thermal cycling, acoustic shearing, and long-term storage in aqueous solution^{20,21}. When amplified, translesion synthesis could occur, introducing a mutation in vitro. These, along with other errors in sample preparation and sequencing, contribute to an error rate of 0.1-1% in NGS²².

Due to the stochasticity of base damage errors, most can be overcome by barcoding and sequencing multiple copies of each DNA fragment and requiring a consensus among reads. Such methods can reduce errors by up to 100-fold, when requiring a consensus from each single strand of DNA, and up to 10,000-fold, when requiring a consensus from both sense strands of each DNA duplex in a technique called duplex sequencing²³. However, most double-stranded DNA fragments, including those which have been sheared for sequencing, have ‘jagged ends’ which must be repaired in order to ligate sequencing adapters to both strands. ‘End Repair/dA-Tailing’ (ER/AT) methods are designed to remove 3′ overhangs, fill-in 5′ overhangs, phosphorylate 5′ ends (via ‘End Repair’), and leave a single dAMP on each 3′ end (via ‘dA-tailing’) to facilitate ligation of dTMP-tailed adapters. Yet, ER/AT methods include polymerases which may resynthesize portions of each duplex.

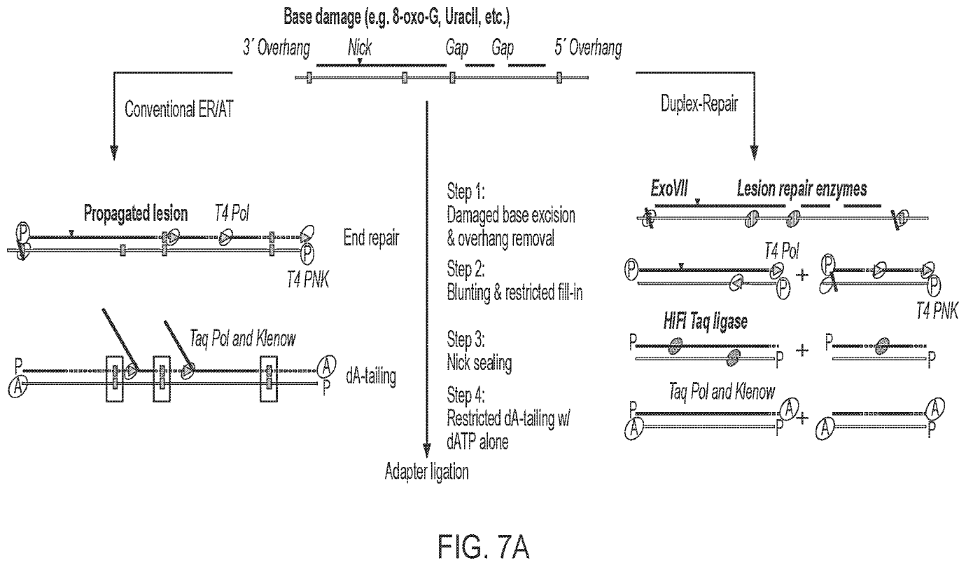

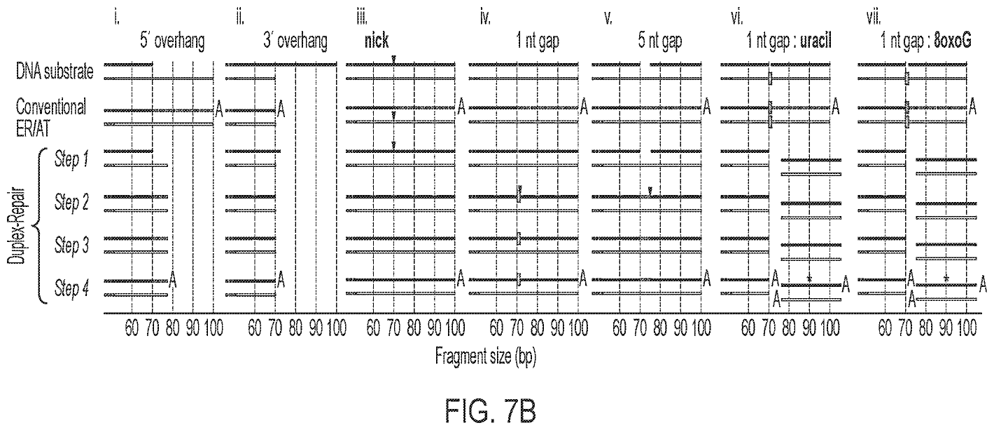

If resynthesis occurs in the presence of an amplifiable lesion or alteration confined to one strand, the altered base pairing will be propagated to the newly synthesized strands when amplified. This will render an amplifiable lesion or alteration from one strand indiscernible from a true mutation on both strands (FIG. 7A). This issue has been observed at the ends of each duplex (e.g. last ˜12 bp) due to fill-in of short 5′ overhangs²⁴. However, such errors could also span much deeper given (i) the 5′ exonuclease and strand-displacement activities of Taq and Klenow polymerases used in ER/AT²⁵ and (ii) the varied nicks, gaps, and overhangs in DNA²⁶ which could act as ‘priming sites’ for strand resynthesis.

It is demonstrated herein that substantial portions of each duplex are resynthesized when conventional ER/AT is applied to DNA bearing nicks, gaps, or overhangs. Also presented herein is a new ER/AT method called Duplex-Repair which limits strand resynthesis. Using single-molecule and panel sequencing, it is shown that Duplex-Repair minimizes strand resynthesis and restores high accuracy despite varied extents of DNA damage, when applied to samples such as cfDNA and formalin-fixed tumor biopsies.

### Methods Related to Example 2

Duplex-Repair workflow: Duplex-Repair consists of four steps. In step 1, DNA is treated with an enzyme cocktail consisting of EndoIV (Cat. No. M0304S), Fpg (Cat. No. M0240S), UDG (Cat. No. M0280S), T4 PDG (Cat. No. M0308S), EndoVIII (Cat. No. M0299S) and ExoVII (Cat. No. M0379S) (all from NEB; use 0.2 uL each) in 1×NEBuffer 2 in the presence of 0.05 ug/uL BSA (total reaction volume=20 uL) at 37° C. for 30 min. In step 2, T4 PNK (Cat. No. M0201S; NEB; use 0.25 uL), T4 DNA polymerase (Cat. No. M0203S; NEB; use 0.25 uL), ATP (final concentration=0.8 mM), and dNTP mix (final concentration of each dNTP=0.5 mM) are added into the step 1 reaction mix and incubated at 37° C. for another 30 min. In step 3, HiFi Taq ligase (Cat. No. M0647S; NEB; use 0.5 uL) and 10× HiFi Taq ligase buffer (use 1.5 uL) are spiked into the step 2 reaction mix and incubated on a thermal cycler that heats from 35° C. to 65° C. over the course of 45 min. The resulting products are purified by performing 3× Ampure bead cleanup and eluted in 17 uL of 10 mM Tris buffer. In step 4, the purified products are treated with Klenow fragment (3′→5′ exo-) (Cat. No. M0212L; NEB; use 1 uL) and Taq DNA polymerase (Cat. No. M0273S; NEB; use 0.2 uL) in 1×NEBuffer 2 in the presence of 0.2 mM dATP (total reaction volume=20 uL) at room temperature for 30 min followed by 65° C. for 30 min. To prepare Duplex-Repair libraries for sequencing, T4 DNA ligase (Cat. No. M0202L; NEB; use 1000 units), 5′-deadenylase (Cat. No. M0331S; NEB; use 0.5 uL), PEG 8000 (final concentration=10% (w/v)), and custom dual index duplex UMI adapters (IDT) are added to the step 4 reaction mix (total reaction volume=55 uL) which is then incubated at room temperature for 1 hr followed by performing 1.2× Ampure bead cleanup, and the purified products are amplified by PCR.

Quantification of strand resynthesis on synthetic oligonucleotides by capillary electrophoresis: fluorophore-labeled single-stranded oligonucleotides (from IDT; Table 1) were resuspended in low TE buffer (pH 8.0) and annealed to form DNA duplexes bearing nicks, gaps, or overhangs. Then, 20-200 ng of each duplex substrate was carried through the workflow of a conventional ER/AT kit, the Kapa Hyper Prep kit, or Duplex-Repair, and aliquots of products after each step were sent to Eton Bioscience for capillary electrophoresis analysis. The returned data were analyzed with Peak Scanner 2 software and then recalibrated.

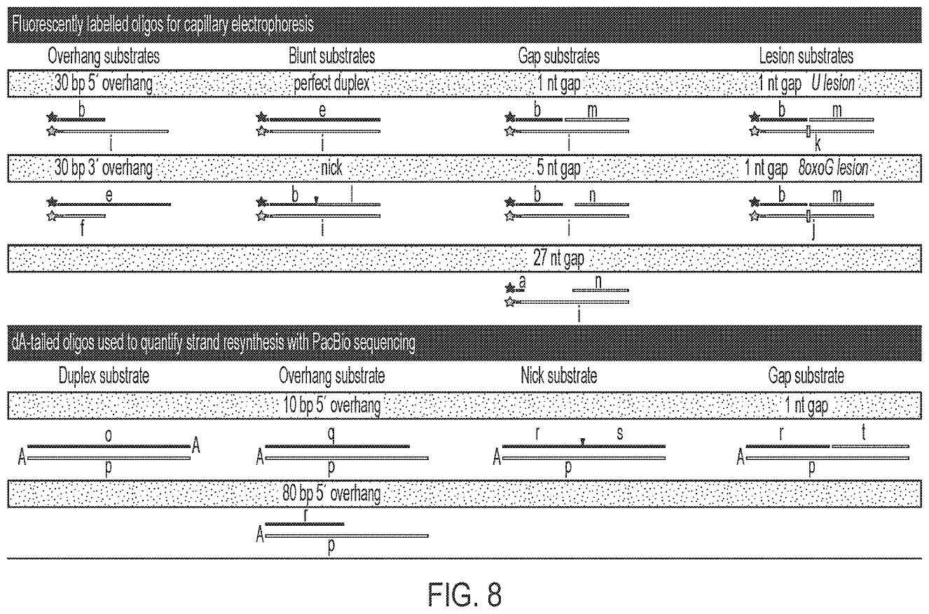

To recalibrate the capillary electrophoresis traces, lengths of synthetic oligonucleotides were confirmed by IDT's mass spectrometry analysis (data not shown). However, the control peak locations reported from raw fragment analysis by using the Peak scanner 2 software differ from the expected positions (Table 1, FIG. 8); the peak locations of 6-FAM tagged molecules consistently appear as underestimates whereas those with ATTO 550 present as overestimates.

TABLE 1
DNA sequences of synthetic oligonucleotides. Asterisks (*) indicate the presence
of a C3 spacer or phosphorothioate bonds that protect fluorophores from being cleaved by
nucleases.

| | | | | | SEQ |
|---|---|---|---|---|---|
| Oligo | Fluorophore | | | Length | ID |
| ID | end | Fluorophore | Sequence | (bp) | NO: |
| a | 5′ | 6-FAM | 6FAM//iSpC3/GCGTCACCAGCCACGCGAGCCGGAT | 48 | 14 |
| | | | GAGGATCCGTGACGCGAAGT | | |
| b | 5′ | 6-FAM | 6FAM//iSpC3/GCGTCACCAGCCACGCGAGCCGGAT | 70 | 15 |
| | | | GAGGATCCGTGACGCGAAGTCCTGGTACCGCCGC | | |
| | | | TCGCTTCCGAC | | |
| c | 5′ | 6-FAM | 6FAM//iSpC3/GCGTCACCAGCCACGCGAGCCGGAT | 80 | 16 |
| | | | GAGGATCCGTGACGCGAAGTCCTGGTACCGCCGC | | |
| | | | TCGCTTCCGACCGGTTCTCCA | | |
| d | 5′ | 6-FAM | 6FAM//iSpC3/GCGTCACCAGCCACGCGAGCCGGAT | 90 | 17 |
| | | | GAGGATCCGTGACGCGAAGTCCTGGTACCGCCGC | | |
| | | | TCGCTTCCGACCGGTTCTCCACCGAGCGACC | | |
| e | 5′ | 6-FAM | 6FAM//iSpC3/GCGTCACCAGCCACGCGAGCCGGAT | 100 | 18 |
| | | | GAGGATCCGTGACGCGAAGTCCTGGTACCGCCGC | | |
| | | | TCGCTTCCGACCGGTTCTCCACCGAGCGACCTAAT | | |
| | | | ATTAAT | | |
| f | 3′ | ATTO 550 | GTCGGAAGCGAGCGGCGGTACCAGGACTTCGCGT | 70 | 19 |
| | | | CACGGATCCTCATCCGGCTCGCGTGGCTGGTGACG | | |
| | | | C/iSpC3//3ATTO550 | | |
| g | 3′ | ATTO 550 | TGGAGAACCGGTCGGAAGCGAGCGGCGGTACCAG | 80 | 20 |
| | | | GACTTCGCGTCACGGATCCTCATCCGGCTCGCGTG | | |
| | | | GCTGGTGACGC/iSpC3//3ATTO550 | | |
| h | 3′ | ATTO 550 | GGTCGCTCGGTGGAGAACCGGTCGGAAGCGAGCG | 90 | 21 |
| | | | GCGGTACCAGGACTTCGCGTCACGGATCCTCATCC | | |
| | | | GGCTCGCGTGGCTGGTGACGC/iSpC3//3ATTO550 | | |
| i | 3′ | ATTO 550 | ATTAATATTAGGTCGCTCGGTGGAGAACCGGTCG | 100 | 22 |
| | | | GAAGCGAGCGGCGGTACCAGGACTTCGCGTCACG | | |
| | | | GATCCTCATCCGGCTCGCGTGGCTGGTG*A*C*G*C/ | | |
| | | | 3ATTO550 (*phosphorothioate bonds) | | |
| j | 3′ | ATTO 550 | ATTAATATTAGGTCGCTCGGTGGAGAACC/i8oxodG | 100 | 23 |
| | | | /GTCGGAAGCGAGCGGCGGTACCAGGACTTCGCGT | | |
| | | | CACGGATCCTCATCCGGCTCGCGTGGCTGGTGA*C | | |
| | | | *G*C*/3ATTO550N/ (*phosphorothioate bonds) | | |
| k | 3′ | ATTO 550 | ATTAATATTAGGTCGCTCGGTGGAGAACCUGTCG | 100 | 24 |
| | | | GAAGCGAGCGGCGGTACCAGGACTTCGCGTCACG | | |
| | | | GATCCTCATCCGGCTCGCGTGGCTGGTGA*C*G*C*/ | | |
| | | | 3ATTO550N/ (*phosphorothioate bonds) | | |
| l | / | / | CGGTTCTCCACCGAGCGACCTAATATTAAT | 30 | 25 |
| m | / | / | GGTTCTCCACCGAGCGACCTAATATTAAT | 29 | 26 |
| n | / | / | CTCCACCGAGCGACCTAATATTAAT | 25 | 27 |
| o | / | / | GTCAAGGGTAATGGACAGTAGGTGTGGTGGAACA | 166 | 28 |
| | | | TACTTCCAGCACTCAAGAAGCTGAAGCAGGCAGA | | |
| | | | TCTCTGTCAGTTCATGACCACTGCTGTCTACATGG | | |
| | | | TGAGCTCCAAGCCAGCCAGGCAAGAAGTGACACT | | |
| | | | CAGGtCTCGCATTGCTcagACGgCaggcA | | |
| p | / | / | TgcctGcCGTctgAGCAATGCGAGaCCTGAGTGTCACT | 166 | 29 |
| | | | TCTTGCCTGGCTGGCTTGGAGCTCACCATGTAGAC | | |
| | | | AGCAGTGGTCATGAACTGACAGAGATCTGCCTGC | | |
| | | | TTCAGCTTCTTGAGTGCTGGAAGTATGTTCCACCA | | |
| | | | CACCTACTGTCCATTACCCTTGACA | | |
| q | / | / | GTCAAGGGTAATGGACAGTAGGTGTGGTGGAACA | 156 | 30 |
| | | | TACTTCCAGCACTCAAGAAGCTGAAGCAGGCAGA | | |
| | | | TCTCTGTCAGTTCATGACCACTGCTGTCTACATGG | | |
| | | | TGAGCTCCAAGCCAGCCAGGCAAGAAGTGACACT | | |
| | | | CAGGtCTCGCATTGCTcag | | |
| r | / | / | GTCAAGGGTAATGGACAGTAGGTGTGGTGGAACA | 86 | 31 |
| | | | TACTTCCAGCACTCAAGAAGCTGAAGCAGGCAGA | | |
| | | | TCTCTGTCAGTTCATGAC | | |
| s | / | / | CACTGCTGTCTACATGGTGAGCTCCAAGCCAGCCA | 80 | 32 |
| | | | GGCAAGAAGTGACACTCAGGtCTCGCATTGCTcagA | | |
| | | | CGgCaggcA | | |
| t | / | / | ACTGCTGTCTACATGGTGAGCTCCAAGCCAGCCA | 79 | 33 |
| | | | GGCAAGAAGTGACACTCAGGtCTCGCATTGCTcagA | | |
| | | | CGgCaggcA | | |

To interpret the capillary electrophoresis data, the peak locations were recalibrated by using a ladder of synthetic oligonucleotides with known lengths. Equations 1-2 relates the oligonucleotide length to raw peak locations through linear regression.

y=1.0381x−7.681 Eq.1

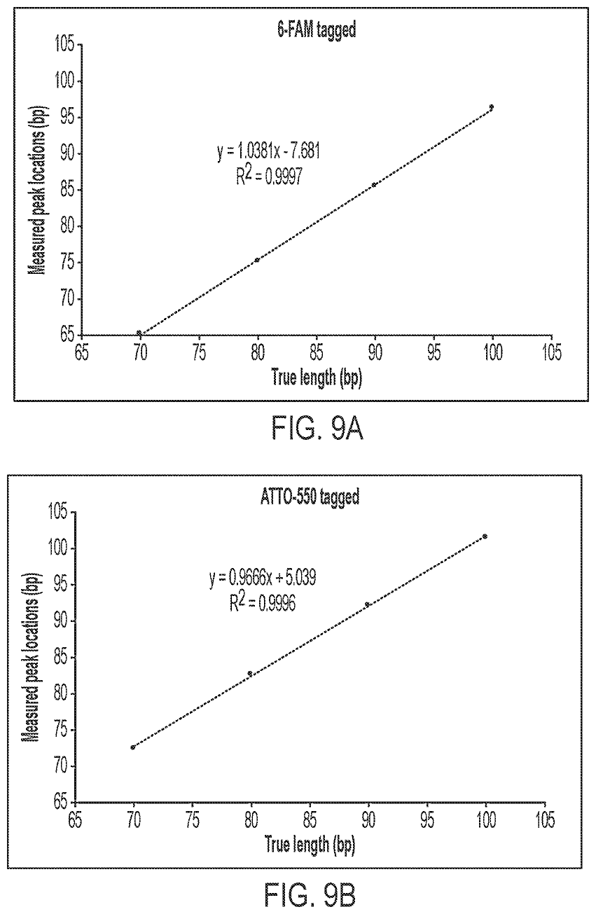

Equation 1. Linear regression of raw fragment analysis peak locations of the 6-FAM-tagged strands. Experimentally determined values for the oligos tagged with 6-FAM in the 100 bp, 90 bp, 80 bp and 70 bp ssDNA controls (Table 1 oligos e, d, c, b respectively) were used to generate a model that relates actual oligonucleotide length (x) to the fragment analysis readout (y) for 6-FAM substrates (FIG. 9A).

y=0.9666x+5.039 Eq. 2

Equation 2. Linear regression of raw fragment analysis peak locations of the ATTO 550-tagged strands. Experimentally determined values for the oligos tagged with ATTO-550 in the 100 bp, 90 bp, 80 bp and 70 bp ssDNA controls (Table 1 oligos i, h, g, f respectively) were used to generate a model that relates actual oligonucleotide length (x) to the fragment analysis readout (y) for ATTO-550 substrates (FIG. 9B).

Clinical specimens. All patients provided written informed consent to allow the collection of blood and/or tumor tissue and analysis of genetic data for research purposes. Healthy donor blood samples were ordered from Research Blood Components or Boston Biosciences. Patients with metastatic breast cancer were prospectively identified for enrollment into an IRB-approved tissue analysis and banking cohort (Dana-Farber Cancer Institute [DFCI] protocol identifier 05-055). Plasma was derived from 10-20 cc whole blood in EDTA tubes.

Quantification of strand resynthesis on cfDNA or gDNA by PacBio sequencer: PacBio's workflow was followed to prepare multiplexed libraries by using the SMRTbell express template kit 2.0 (Pacific Biosciences) but made these modifications: 1). “Remove SS overhangs” and “DNA damage repair” steps were skipped; 2) ER/AT was performed by using the Kapa Hyper Prep kit or Duplex-Repair; 3). A custom buffer (5×) was prepared, consisting of 250 mM Tris, 2 mM d^{6m} ATP, 2 mM d^{4m}CTP, 2 mM dGTP, 2 mM dTTP, 50 mM MgCl₂, 50 mM DTT, and 5 mM ATP (pH 7.5), and was used to perform ER/AT with d^{6m}ATP (N6-methyl-2′-deoxyadenosine-5′-triphosphate), d^{4m}CTP (N4-methyl-2′-deoxycytidine-5′-triphosphate), dGTP, and dTTP (all from TriLink Biotechnologies); 4). 1.8× Ampure PB bead cleanup was performed after nuclease treatment; 5). The “Second Ampure PB bead purification” step was skipped. The input into each library construction was 50 ng of a synthetic oligonucleotide or 20-40 ng of cfDNA or gDNA. As-prepared PacBio libraries were sequenced on Sequel II with a targeted read count of at least 65000 per sample.

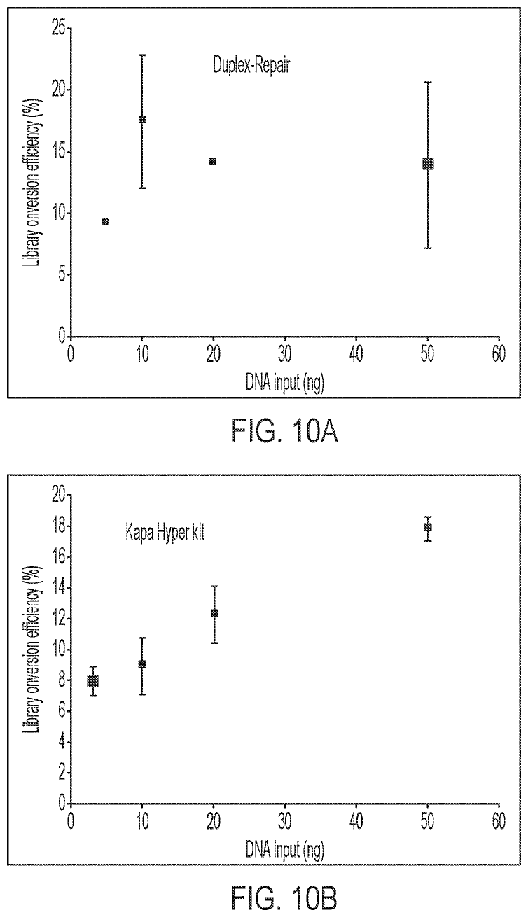

Induction of DNA damage by CuCl₂/H₂O₂ and DNase I. The conditions for inducing DNA damage were optimized by CuCl₂/H₂O₂ and DNase I (FIGS. 10A-10B, FIG. 11, FIG. 12, Table 2). Then, 20 ng of cfDNA was treated with 0, 0.2, or 2 mU DNase 1 (Cat. No. M0303S; NEB) and 0, 1, or 100 uM CuCl₂/H₂O₂ in 1× DNase 1 buffer (total reaction volume=20 uL) at 16° C. for 1 hr. 40 mM EDTA was then added to quench the reaction, and the resulting products were purified by performing a 2× Ampure bead cleanup.

TABLE 2
Quantification of DNA loss after DNase 1 treatment.
The input was 20 ng of a 100 bp dsDNA oligo.

| | Yields of DNA products after |
|---|---|
| DNase I | DNase 1 treatment, mean ± SD |
| amount (mU) | (with two biological replicates) |
| 0 | 2.48 ± 0.32* |
| 0.02 | 2.48 ± 0.04 |
| 0.2 | 2.18 ± 0.12 |
| 2 | 2.06 ± 0.04 |
| 20 | 1** |

*the low yield indicates a significant loss during the Ampure bead cleanup step;
**the concentration of the 2nd biological replicate is below the detection limit of the Qubit assay.

Processing of cfDNA sample and gDNA sample: cfDNA was extracted from fresh or archival plasma of healthy donors or cancer patients by following the same method as before^{24,27}. gDNA was extracted from FFPE tumor tissues or buffy coats, sheared and quantified by following the same protocol as previously described^{24,27}. Then, cfDNA or gDNA libraries were constructed from 10-20 ng DNA inputs by using the Kapa Hyper Prep kit or Duplex-Repair with custom dual index duplex UMI adapters (IDT). Hybrid Selection (HS) using IDT's pan-cancer panel was performed on the prepared libraries using the xGen hybridization and wash kit with xGen Universal blockers (IDT). After the second round of HS, libraries were amplified, quantified and pooled for sequencing on a HiSeq 2500 rapid run (100 bp paired-end runs) or HiSeqX (151 bp paired-end runs) with a targeted raw depth of 200,000× per site.

Analysis of duplex sequencing data and quantification of error rates: Raw reads were then processed through the duplex consensus calling pipeline as previously described²⁴. Error rates were calculated by counting the proportion of non-reference bases to total bases after applying filters specifically tailored to duplex sequencing²⁴. To avoid miscounting true somatic variants from cancer patients as base errors, any loci that had a somatic mutation were omitted from whole exome sequencing of that patient's tumor biopsy. A matched normal derived from buffy coat DNA was also used to filter any germline mutations. For base error position analysis, the error metrics collection pipeline was rerun with the end of fragment filter disabled to observe errors across the entire DNA duplex.

Estimating resynthesis from single molecule real-time (SMRT) sequencing data: First, the Circular Consensus Sequences (CCS) tool (Pacific Biosciences) was used to generate consensus reads from the raw reads. The—mean-kinetics flag was also used to output interpulse durations (IPDs), among other metrics, for each base position to be used later for identifying modified dNTPs. The lima tool (Pacific Biosciences) was then used to demultiplex the samples that were sequenced together on the same flow cell. These CCS reads were then used as input for the Hidden Markov Model (HMM) to estimate strand resynthesis.

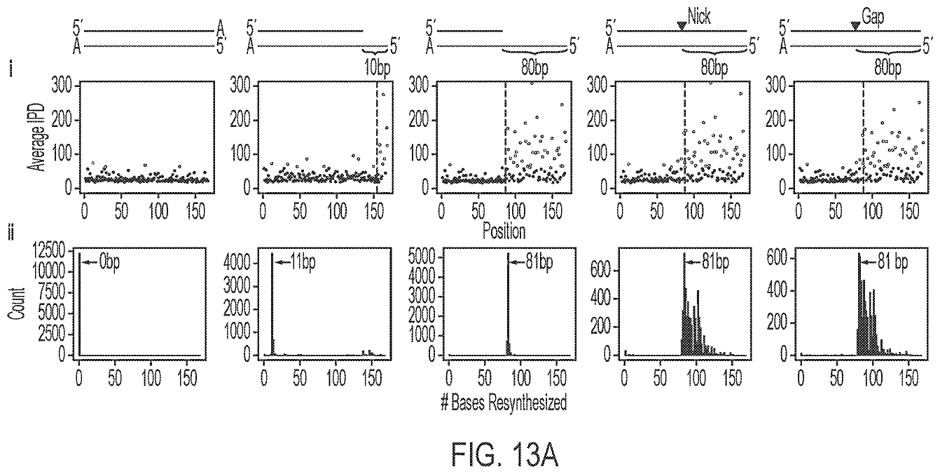

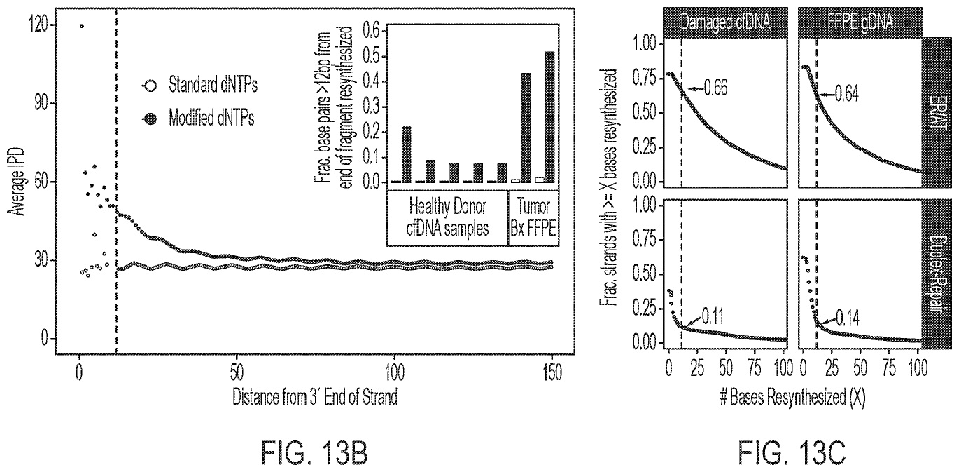

A HMM was then implemented to estimate the amount of resynthesis on the 3′ end of each duplex strand from SMRT sequencing data. The HMM consists of two states that represent regions with original bases (O) and regions with bases that were filled-in during ER/AT (F) respectively. The HMM was designed to estimate resynthesis that starts at an interior position in the strand and continues all the way to the 3′ end. In addition, a transition matrix that does not allow F to O transitions was designed. The transition probability from O to F, x, equal to the reciprocal of the strand length and the transition probability from O to O, y equal 1−x. To develop an empirical emission matrix, synthetic duplexes were sequenced with known regions of resynthesis and of original bases (Table 1). PacBio SMRT sequencing emits both the base and interpulse duration (IPD) for each position which were then collected to form the emission matrix of IPD distributions for each base in each state (FIG. 13A-13C). Using this HMM, the Viterbi algorithm was applied to each duplex DNA strand to determine the most likely regions of original bases and of resynthesized bases and the total number of resynthesized bases was calculated.

To estimate the fraction of interior base pair resynthesized, the regions of estimated resynthesis from the HMM was taken and the number of resynthesized base pairs that were greater than 12 base pairs from either end of the duplex fragment relative to the total number of base pairs that were greater than 12 base pairs from either fragment end was counted. For all analyses control samples were also run with standard, non-modified dNTPs to measure the background resynthesis estimates and subtracted that background from the samples where modified dNTPs were used.

### Duplex-Repair as a New ER/AT Approach

To determine if conventional ER/AT methods could resynthesize substantial portions of DNA duplexes bearing nicks, gaps, or overhangs, including those with amplifiable lesions, duplex oligonucleotides bearing (i) 5′ overhangs, (ii) 3′ overhangs, (iii) nicks, (iv-v) gaps of varied lengths without base damage, or (vi-vii) gaps with base damage (FIG. 7B, Table 1) were generated. The top and bottom strands were labeled with different dyes so that capillary electrophoresis could be used to quantify changes in fragment length during ER/AT (FIGS. 7A-7B).

To quantify library conversion efficiency, a ddPCR assay was designed to target the flanking adapter regions. Only fragments with successful double ligation were exponentially amplified within the QX200 ddPCR EvaGreen Supermix (Bio-Rad) and thus detected.

ddPCR assay design

Primer 1: CACTCTTTCCCTACACGACG (SEQ ID NO: 1)
Primer 2: AGTTCAGACGTGTGCTCTTC (SEQ ID NO: 2)

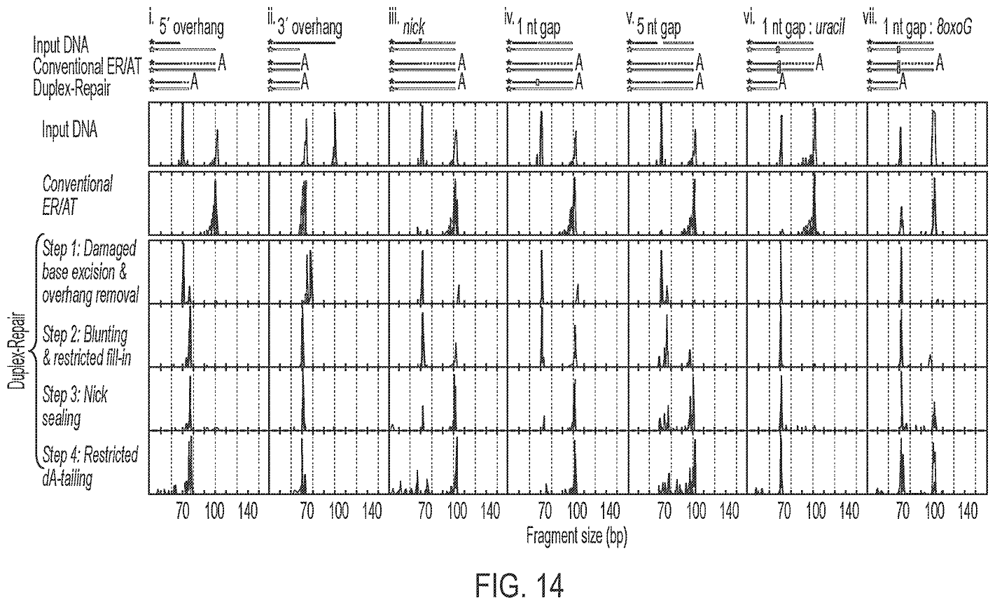

Conventional ER/AT methods were applied and substantial strand resynthesis was observed in all substrates except for those with 3′ overhangs (FIG. 7B, FIG. 14). For instance, with even just a single nick in the middle of the top strand, the 30 bases downstream of the nick site were entirely resynthesized. These results confirm conventional ER/AT methods can resynthesize large portions of each duplex, when nicks, gaps, or overhangs are present.

To address this issue, a new approach called Duplex-Repair was devised, which conducts ER/AT in a careful and stepwise manner to limit strand resynthesis (FIG. 7A). Duplex-Repair was designed to ‘concentrate’ resynthesis at fragment ends (e.g. last 12 bp) where errors can be trimmed in silico²⁴. Duplex-Repair consists of four steps: (1) damaged base excision and overhang removal, (2) blunting and restricted fill-in, (3) nick sealing, and (4) restricted dA-tailing. In step 1, DNA is treated with an enzyme cocktail consisting of enzymes involved in Base Excision Repair (BER), such as Endonuclease IV (EndoIV), Formamidopyrimidine [fapy]-DNA glycosylase (Fpg), Uracil-DNA glycosylase (UDG), T4 pyrimidine DNA glycosylase (T4 PDG), and Endonuclease VIII (EndoVIII). These enzymes excise damaged bases such as Uracil, 8′oxoG, oxidized pyrimidines, cyclobutane pyrimidine dimers and cleave abasic sites, resulting in 1 nt gaps in double-stranded regions or strand breaks in single-strand regions. Exonuclease VII (ExoVII) is also used in this step to degrade 3′ and 5′ single-strand overhangs. Then, in step 2, T4 polynucleotide kinase (de)phosphorylates DNA termini, while T4 DNA polymerase blunts 3′ overhangs and fills in the small gaps and short (≤7 nt) 5′ overhangs which remain after ExoVII digestion. After that, nicks are sealed by HiFi Taq DNA ligase in step 3. In step 4, restricted dA-tailing is performed using Klenow fragment (exo-) and Taq DNA polymerase, but with only dATP present, to limit their activities to non-templated extension.

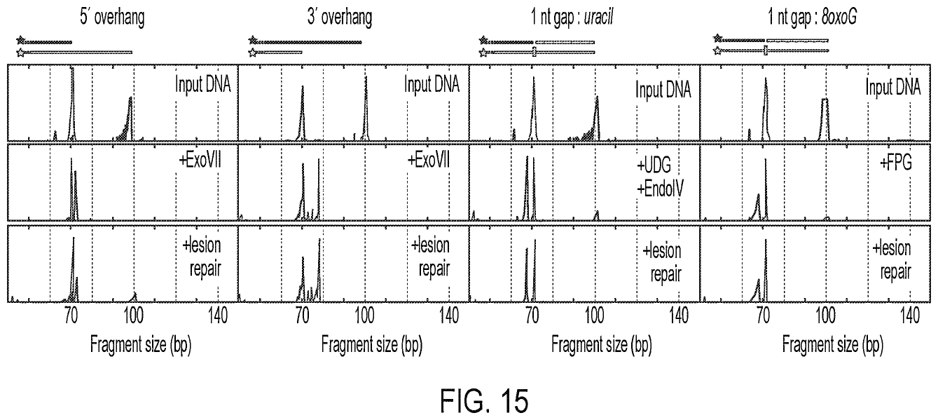

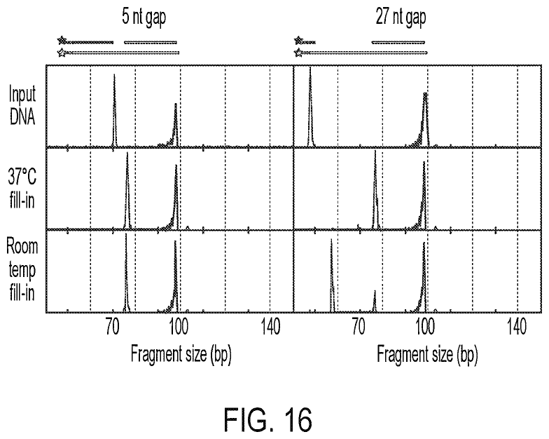

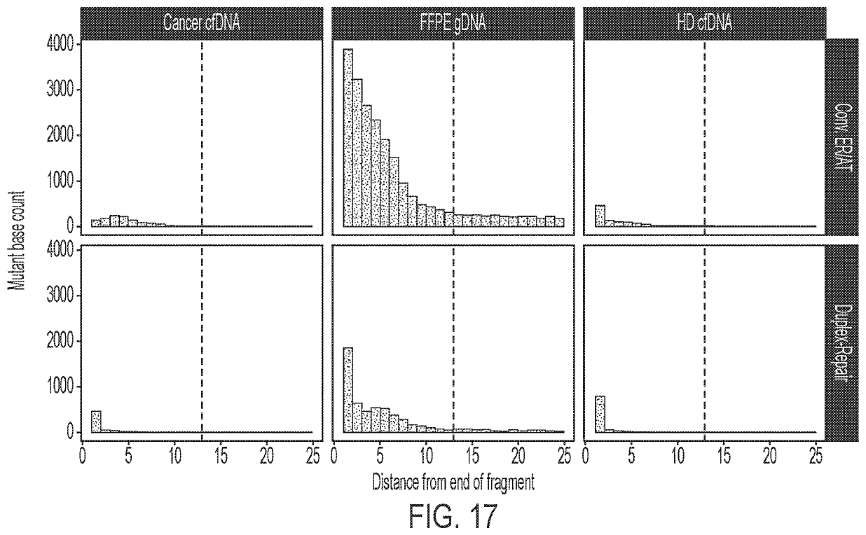

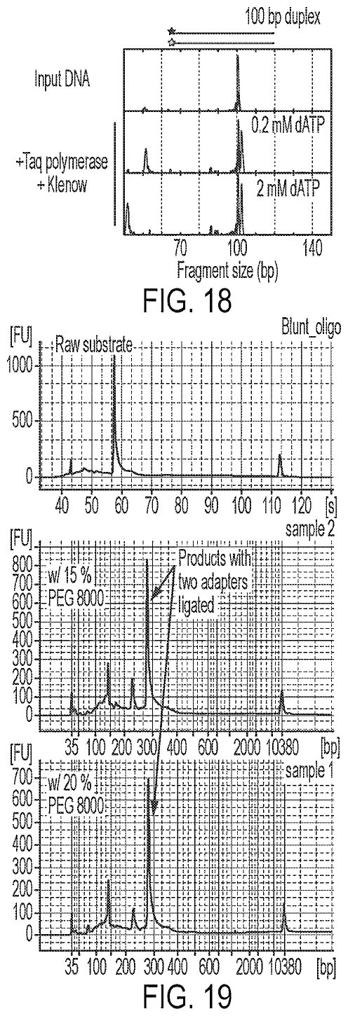

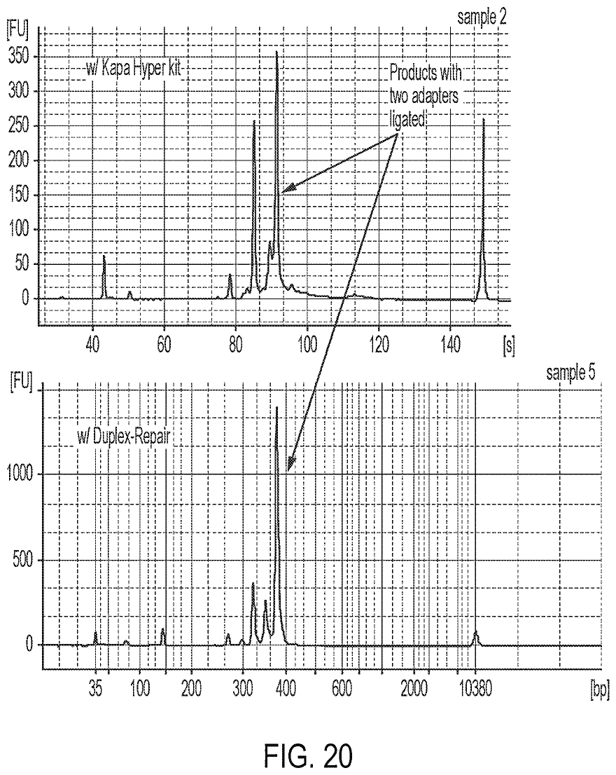

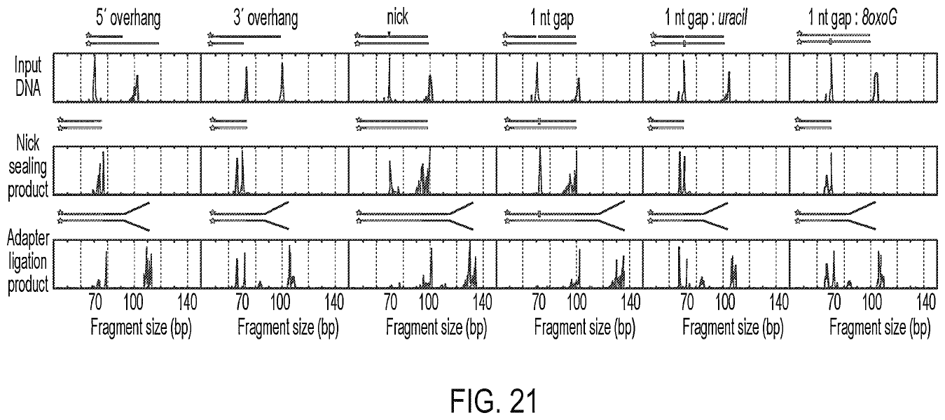

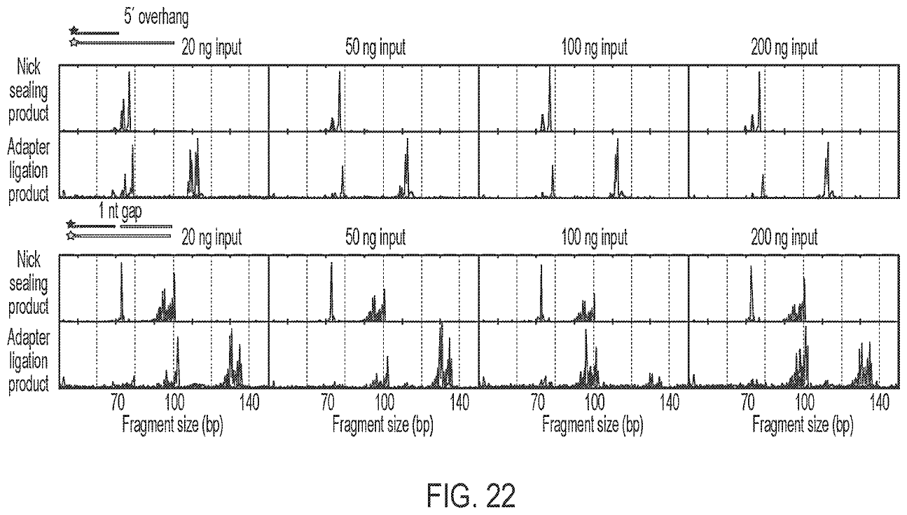

Using the aforementioned synthetic duplexes, it was confirmed that Duplex-Repair facilitates ER/AT with minimal resynthesis. First, each step was tested in ideal buffer conditions by performing a 3× Ampure bead cleanup after each step and have depicted the major products (FIG. 7B and FIG. 14). For each substrate, it was confirmed that the activity of the key enzymes involved, while making sure that the other enzymes present did not compromise their activity. For instance, for substrate (i), the long 5′ overhang is largely digested by ExoVII (FIG. 15) while the remaining three bases are filled in by T4 DNA polymerase (FIG. 14). For substrate (ii), the 3′ overhang is digested in part by ExoVII (FIG. 15), and then blunted entirely by T4 DNA polymerase (FIG. 14). For substrate (iii), the nick is sealed by HiFi Taq DNA ligase (FIG. 16). For substrates (iv-v), the gaps are first filled by T4 DNA polymerase (FIG. 14 and FIG. 16) and then the resulting ‘nicks’ are sealed by HiFi Taq DNA ligase. For substrates (vi-vii), the damaged bases are excised (uracil by UDG; 8′oxoG by Fpg; FIG. 17 and FIGS. 13A-13C) and abasic sites cleaved to create strand breaks and thus avoid translesion synthesis during gap filling in step 2. It was also confirmed that dA-tailing works with only dATP present (FIGS. 18-20). The reaction conditions were optimized and multiple Ampure cleanups between steps were eliminated tp help reduce DNA loss (FIGS. 21-22). These results suggest that Duplex-Repair conducts ER/AT in a manner which limits strand resynthesis while achieving comparable library conversion efficiencies as conventional ER/AT (FIG. 10A-10B).

Duplex-Repair Limits Resynthesis of DNA Duplexes from Clinical Specimens

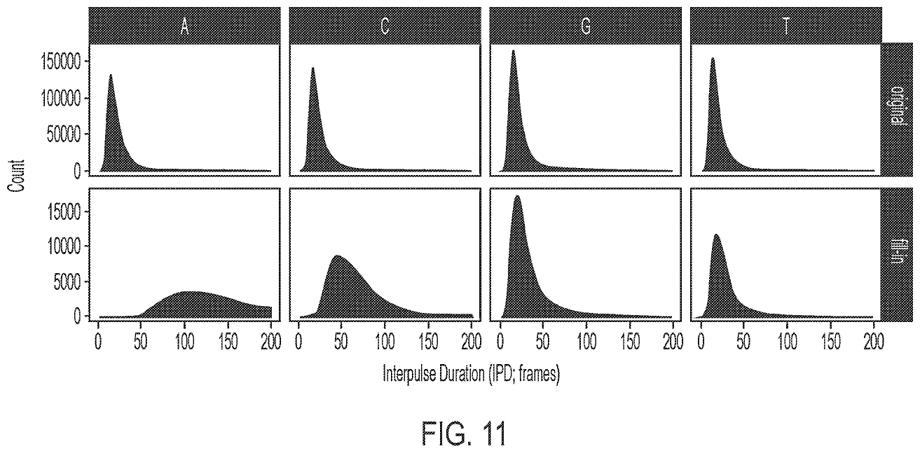

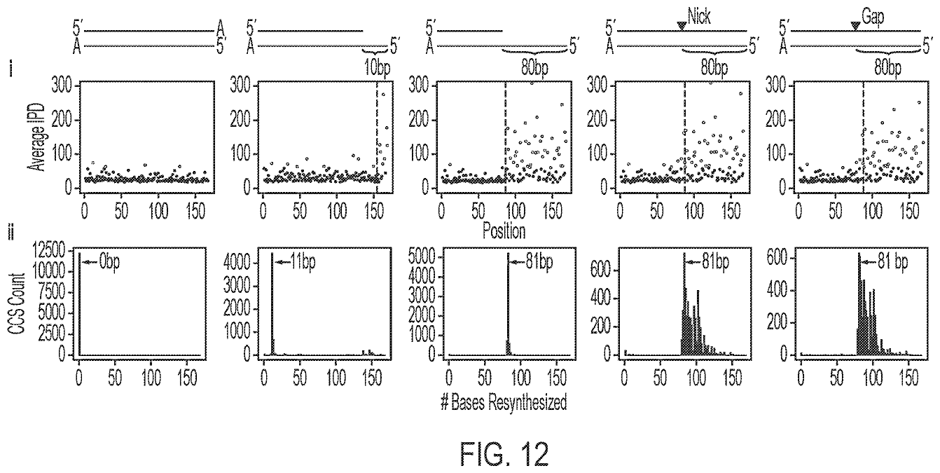

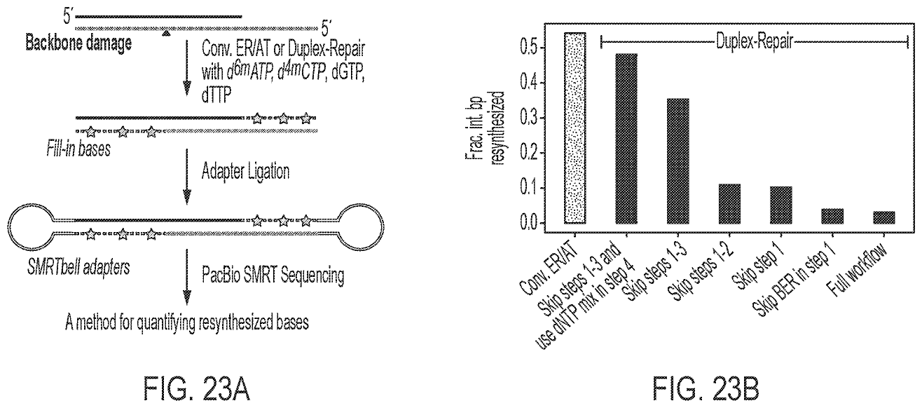

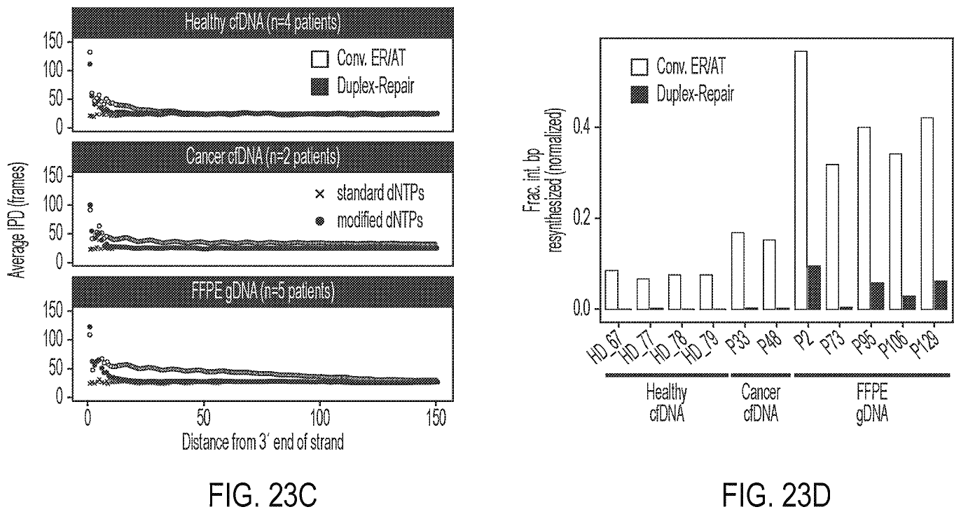

Next, strand resynthesis was quantified when ER/AT was applied to clinical samples such as cell-free DNA (cfDNA) and formalin-fixed paraffin-embedded (FFPE) tumor biopsies. An assay was devised which involved performing ER/AT using a modified dNTP mix comprising d^{6m}ATP, d^{4m}CTP, dTTP, and dGTP, sequencing the prepared libraries on a PacBio sequencer which can detect where d^{6m}ATP and d^{4m}CTP have been incorporated²⁸, and applying a Hidden Markov Model to identify resynthesized regions (FIG. 23A and FIG. 11; Methods). Its performance was verified using synthetic oligonucleotides (Table 1) treated with conventional ER/AT. Extended interpulse durations (IPDs) were observed corresponding to d^{6m}ATP and d^{4m}CTP incorporations in the anticipated regions (FIG. 12, column i). The estimated number of resynthesized bases to be expected in most cases was also found (FIG. 12, column ii). Interestingly, for the substrates with a nick or a gap, some molecules with longer than expected fill-in were found, despite having the same terminal 3′OH as the substrate with a 80 bp 5′ overhang. It is reasoned that this could be due to 3′ exonuclease activity of the polymerase, which may be pronounced when it encounters an adjacent, downstream strand.

Then the above resynthesis quantification method was used to estimate the difference in resynthesized base pairs between Duplex-Repair and conventional ER/AT by testing on a healthy donor cfDNA sample with base and backbone damage induced by 100 uM CuCl₂/H₂O₂ and 2 mU DNase 1, respectively (see Methods). Several variations of Duplex-Repair were also tested in order to assess the impact of each step on limiting resynthesis. Applying this method, it was estimated that 54% of interior duplex base pairs (defined as base pairs that are greater than 12 base pairs from either end of the original duplex DNA fragment) were resynthesized with conventional ER/AT, as compared to 3% with Duplex-Repair (FIG. 23B). Notably, each step in the Duplex-Repair protocol that was tested served to reduce the amount of interior base pair resynthesis further. In particular, it was observed that skipping the BER in step 1 had a negligible impact on resynthesis while skipping step 1 increased interior resynthesis fractions from 3% to 9%, suggesting that ExoVII treatment is required for suppressing resynthesis on 5′ overhangs. Further, skipping step 2 only slightly increased interior resynthesis fractions from 9% to 11%, confirming limited resynthesis occurred during restricted fill-in. Further, skipping step 3 increased interior resynthesis fraction from 11% to 35%, suggesting that unsealed nicks led to significant resynthesis during dA-tailing. Furthermore, using dNTP mix instead of dATP alone in step 4 increased the resynthesis fraction from 35% to 47%, suggesting that it is essential to use dATP alone to suppress templated extension during dA-tailing. Overall, these results suggest that the full protocol of Duplex-Repair is required to minimize resynthesis.

To assess the extent to which Duplex-Repair could limit resynthesis in clinical samples, the assay was used to measure resynthesis across several different sample types, including healthy donor cfDNA, cancer patient cfDNA, and tumor FFPE biopsies.

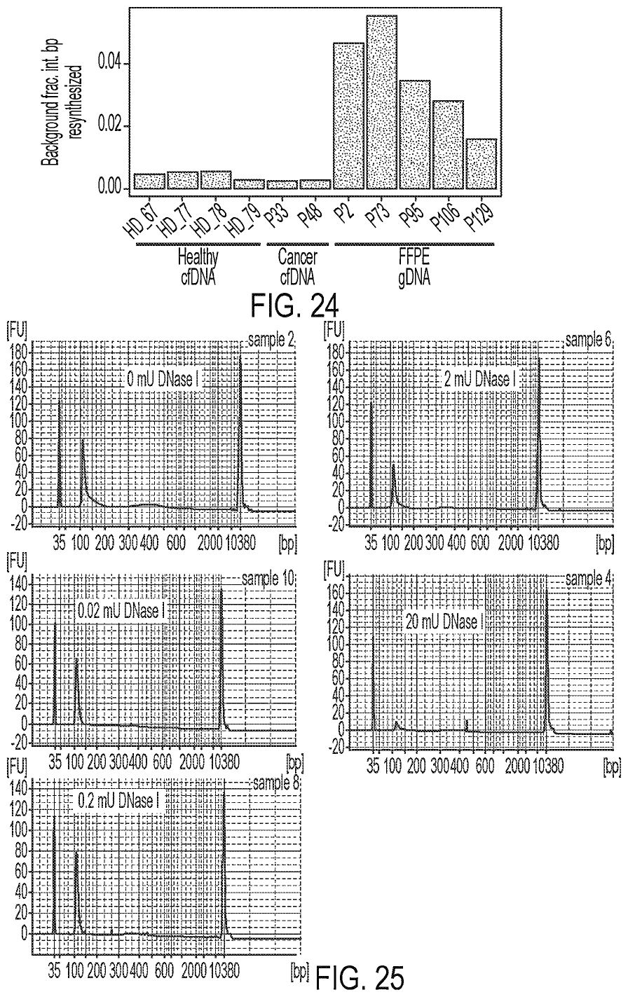

Considering that d^{6m}ATP and d^{4m}CTP could be present as real epigenetic modifications in clinical samples 29, a control sample was also run for each patient using all standard dNTPs and conventional ER/AT to control for any background noise. Average IPDs were looked at across strand positions for each CCS strand relative to the distance from the 3′ end of the original DNA strand (FIG. 23C). For all sample types, consistently low average IPDs were observed across all positions for control samples. In contrast, average IPDs significantly increased both for conventional ER/AT and Duplex-Repair towards the 3′ ends of CCS strands (FIG. 23C). Furthermore, elevated IPDs for Duplex-Repair are concentrated within 12 base pairs from the 3′ end, but they extend much further into the strand for conventional ER/AT. Next the resynthesis quantification method described herein was used to estimate the amount of interior duplex base pair resynthesis in the clinical samples. The fractions of interior base pairs resynthesized (after subtracting out the background noise from the control samples; FIG. 24) are much higher for conventional ER/AT compared to Duplex-Repair across all sample types (FIG. 23D). In particular, it was observed that with conventional ER/AT, on average 8% (range 7-9%), 16% (range 15-17%), and 41% (range 32-57%) of interior duplex base pair resynthesis occurred for healthy cfDNA, cancer patient cfDNA, and FFPE tumor gDNA samples, respectively, which decreased to 0.12% (range 0.00-0.17%), 0.0345% (range 0.03-0.04%), and 5% (range 0.5-10%) when Duplex-Repair was used and thus corresponded to reductions in interior base pair resynthesis of 67-fold, 464-fold, and 8-fold. These results suggest that conventional ER/AT induces substantial strand resynthesis in clinical samples such as cfDNA and FFPE tumor biopsies and that Duplex-Repair can significantly limit this.

### Duplex-Repair Overcomes Induced DNA Damage and Enhances Duplex Sequencing

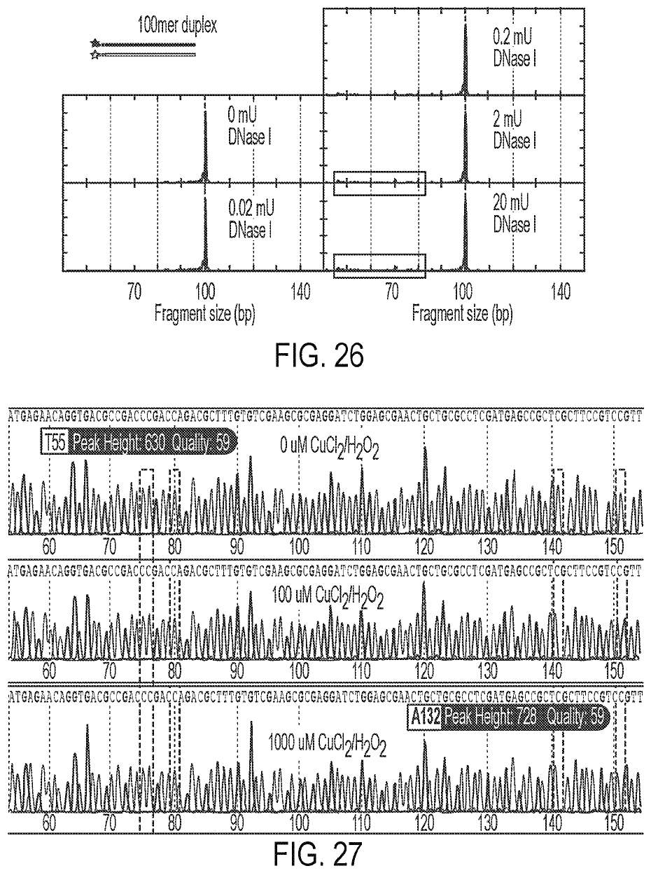

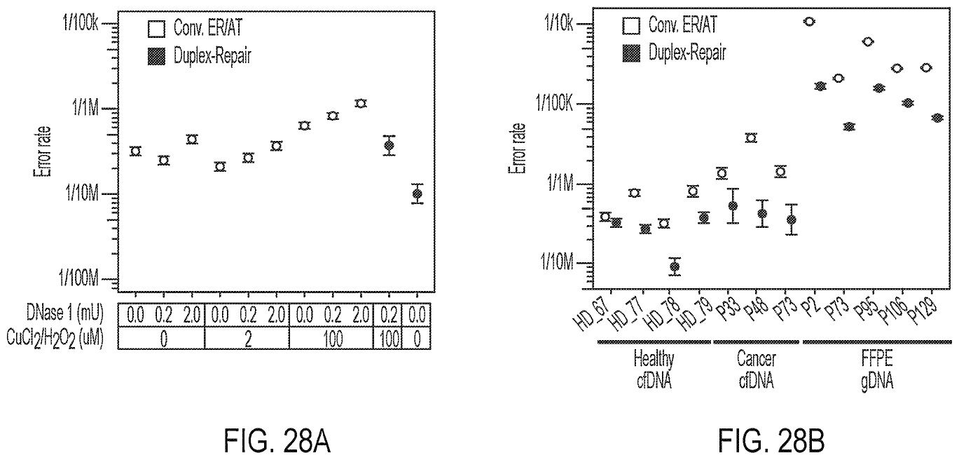

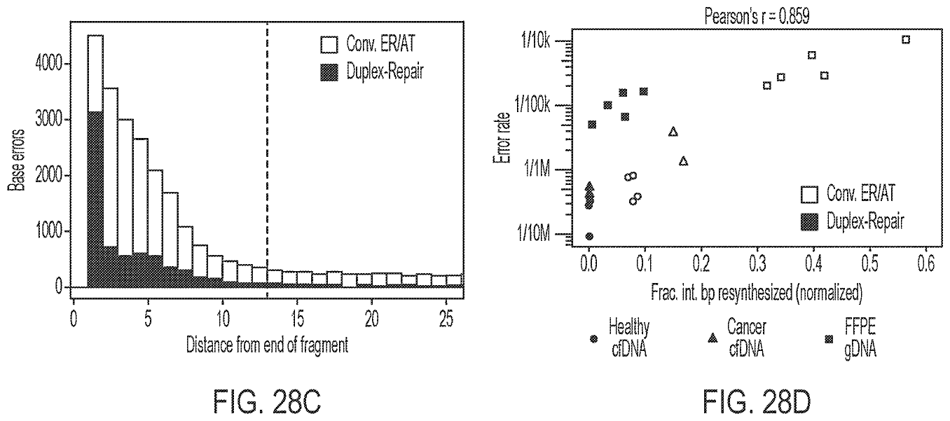

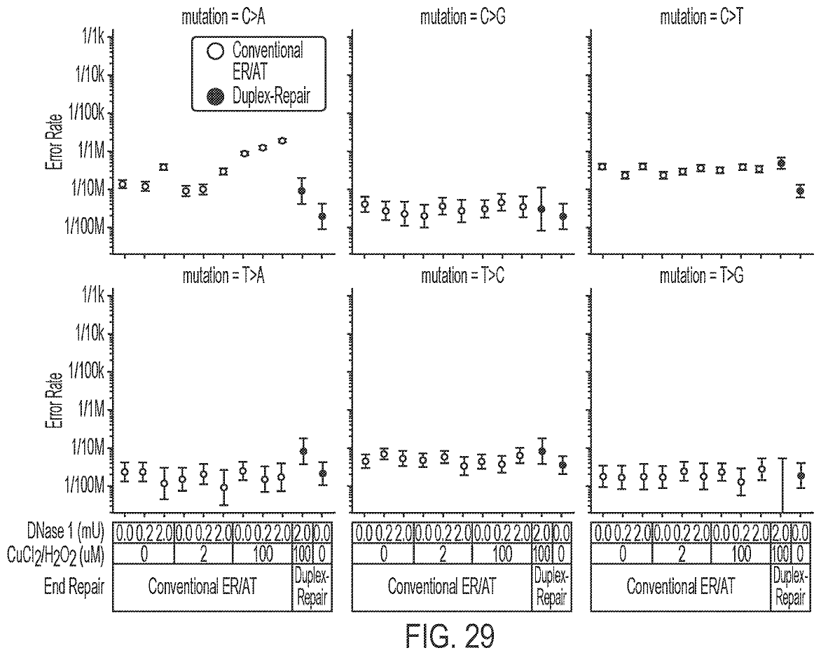

Reasoning that strand resynthesis in ER/AT would be most problematic when amplifiable lesions or alterations are present, cfDNA from one healthy donor (HD 78) was subjected to different concentrations of the oxidizing agent CuCl₂/H₂O₂, and DNase I to induce base and backbone damage without appreciably degrading DNA (FIGS. 25-27, Table 2). Conventional ER/AT was then applied, duplex sequencing was performed, and error rates were computed after trimming the last 12 bp from the ends of each duplex 24 (FIG. 28A, FIG. 29, Table 4).

TABLE 4
Sequencing metrics for all samples profiled by targeted panel sequencing.

| | | | | | | Number | | | |
|---|---|---|---|---|---|---|---|---|---|
| | | | | Number | On | of duplex | Number | | |
| | | | ER/AT | of raw | target | bases | of base | | |
| Specimen | Patient ID | DNA damage inducers | method | reads | rates | evaluated | errors | Error Rate | CI (95%) |
| cfDNA | HD_78 | 0 μM_CuCl₂/ | Conv. | 129340676 | 0.98 | 12216528 | 1 | 8.19E−08 | 1.44e−08- |
| | | H₂O₂ + 0 mU_DNase1 | ER/AT | | | | | | 4.64e−07 |
| cfDNA | HD_78 | 0 μM_CuCl₂/ | Conv. | 154329922 | 0.98 | 10417986 | 3 | 2.88E−07 | 9.79e−08- |
| | | H₂O₂ + 0.2 mU_DNase1 | ER/AT | | | | | | 8.47e−07 |
| cfDNA | HD_78 | 0 μM_CuCl₂/ | Conv. | 138532100 | 0.98 | 9406423 | 5 | 5.32E−07 | 2.27e−07- |
| | | H₂O₂ + 2 mU_DNase1 | ER/AT | | | | | | 1.24e−06 |
| cfDNA | HD_78 | 2 μM_CuCl₂/ | Conv. | 157876394 | 0.98 | 10542247 | 4 | 3.79E−07 | 1.48e−07- |
| | | H₂O₂ + 0 mU_DNase1 | ER/AT | | | | | | 9.76e−07 |
| cfDNA | HD_78 | 2 μM_CuCl₂/ | Conv. | 136344260 | 0.98 | 11439892 | 5 | 4.37E−07 | 1.87e−07- |
| | | H₂O₂ + 0.2 mU_DNase1 | ER/AT | | | | | | 1.02e−06 |
| cfDNA | HD_78 | 2 μM_CuCl₂/ | Conv. | 184586652 | 0.98 | 7559620 | 5 | 6.61E−07 | 2.83e−07- |
| | | H₂O₂ + 2 mU_DNase1 | ER/AT | | | | | | 1.55e−06 |
| cfDNA | HD_78 | 100 μM_CuCl₂/ | Conv. | 133322464 | 0.98 | 11668760 | 5 | 4.28E−07 | 1.83e−07- |
| | | H₂O₂ + 0 mU_DNase1 | ER/AT | | | | | | 1.00e−06 |
| cfDNA | HD_78 | 100 μM_CuCl₂/ | Conv. | 147166330 | 0.98 | 10624035 | 7 | 6.59E−07 | 3.19e−07- |
| | | H₂O₂ + 0.2 mU_DNase1 | ER/AT | | | | | | 1.36e−06 |
| cfDNA | HD_78 | 100 μM_CuCl₂/ | Conv. | 166040642 | 0.98 | 8380284 | 7 | 8.35E−07 | 4.05e−07- |
| | | H₂O₂ + 2 mU_DNase1 | ER/AT | | | | | | 1.72e−06 |
| cfDNA | HD_78 | 0 μM_CuCl₂/ | Conv. | 150545198 | 0.98 | 13171944 | 3 | 2.28E−07 | 7.75e−08- |
| | | H₂O₂ + 0 mU_DNase1 | ER/AT | | | | | | 6.70e−07 |
| cfDNA | HD_78 | 0 μM_CuCl₂/ | Conv. | 129869348 | 0.98 | 11141949 | 3 | 2.69E−07 | 9.16e−08- |
| | | H₂O₂ + 0.2 mU_DNase1 | ER/AT | | | | | | 7.92e−07 |
| cfDNA | HD_78 | 0 μM_CuCl₂/ | Conv. | 166455246 | 0.98 | 10042644 | 6 | 5.97E−07 | 2.74e−07- |
| | | H₂O₂ + 2 mU_DNase1 | ER/AT | | | | | | 1.30e−06 |
| cfDNA | HD_78 | 2 μM_CuCl₂/ | Conv. | 120148202 | 0.98 | 10956968 | 1 | 9.13E−08 | 1.61e−08- |
| | | H₂O₂ + 0 mU_DNase1 | ER/AT | | | | | | 5.17e−07 |
| cfDNA | HD_78 | 2 μM_CuCl₂/ | Conv. | 167055596 | 0.98 | 10056176 | 2 | 1.99E−07 | 5.45e−08- |
| | | H₂O₂ + 0.2 mU_DNase1 | ER/AT | | | | | | 7.25e−07 |
| cfDNA | HD_78 | 2 μM_CuCl₂/ | Conv. | 195036858 | 0.98 | 8965981 | 3 | 3.35E−07 | 1.14e−07- |
| | | H₂O₂ + 2 mU_DNase1 | ER/AT | | | | | | 9.84e−07 |
| cfDNA | HD_78 | 100 μM_CuCl₂/ | Conv. | 130254538 | 0.98 | 12904331 | 3 | 2.32E−07 | 7.91e−08- |
| | | H₂O₂ + 0 mU_DNase1 | ER/AT | | | | | | 6.84e−07 |
| cfDNA | HD_78 | 100 μM_CuCl₂/ | Conv. | 130025566 | 0.98 | 10159501 | 6 | 5.91E−07 | 2.71e−07- |
| | | H₂O₂ + 0.2 mU_DNase1 | ER/AT | | | | | | 1.29e−06 |
| cfDNA | HD_78 | 100 μM_CuCl₂/ | Conv. | 152338894 | 0.98 | 6953914 | 7 | 1.01E−06 | 4.88e−07- |
| | | H₂O₂ + 2 mU_DNase1 | ER/AT | | | | | | 2.08e−06 |
| cfDNA | HD_78 | 0 μM_CuCl₂/ | Conv. | 149178894 | 0.98 | 12742674 | 4 | 3.14E−07 | 1.22e−07- |
| | | H₂O₂ + 0 mU_DNase1 | ER/AT | | | | | | 8.07e−07 |
| cfDNA | HD_78 | 0 μM_CuCl₂/ | Conv. | 139004134 | 0.98 | 12832585 | 3 | 2.34E−07 | 7.95e−08- |
| | | H₂O₂ + 0.2 mU_DNase1 | ER/AT | | | | | | 6.87e−07 |
| cfDNA | HD_78 | 0 μM_CuCl₂/ | Conv. | 147683652 | 0.98 | 6998068 | 2 | 2.86E−07 | 7.84e−08- |
| | | H₂O₂ + 2 mU_DNase1 | ER/AT | | | | | | 1.04e−06 |
| cfDNA | HD_78 | 2 μM_CuCl₂/ | Conv. | 153068478 | 0.98 | 13027274 | 0 | 0 | 0.00e+00- |
| | | H₂O₂ + 0 mU_DNase1 | ER/AT | | | | | | 2.95e−07 |
| cfDNA | HD_78 | 2 μM_CuCl₂/ | Conv. | 153781318 | 0.98 | 11278606 | 1 | 8.87E−08 | 1.57e−08- |
| | | H₂O₂ + 0.2 mU_DNase1 | ER/AT | | | | | | 5.02e−07 |
| cfDNA | HD_78 | 2 μM_CuCl₂/ | Conv. | 181927842 | 0.98 | 8682012 | 5 | 5.76E−07 | 2.46e−07- |
| | | H₂O₂ + 2 mU_DNase1 | ER/AT | | | | | | 1.35e−06 |
| cfDNA | HD_78 | 100 μM_CuCl₂/ | Conv. | 136796244 | 0.98 | 11267015 | 5 | 4.44E−07 | 1.90e−07- |
| | | H₂O₂ + 0 mU_DNase1 | ER/AT | | | | | | 1.04e−06 |
| cfDNA | HD_78 | 100 μM_CuCl₂/ | Conv. | 132555006 | 0.98 | 8874034 | 3 | 3.38E−07 | 1.15e−07- |
| | | H₂O₂ + 0.2 mU_DNase1 | ER/AT | | | | | | 9.94e−07 |
| cfDNA | HD_78 | 100 μM_CuCl₂/ | Conv. | 127356706 | 0.98 | 7319479 | 5 | 6.83E−07 | 2.92e−07- |
| | | H₂O₂ + 2 mU_DNase1 | ER/AT | | | | | | 1.60e−06 |
| cfDNA | damaged_HD_78_cfDNA | 100 μM_CuCl₂/ | Duplex- | 53358056 | 0.99 | 44538494 | 13 | 2.92E−07 | 1.71e−07- |
| | | H₂O_{2—}+_2 mU_DNase1 | Repair | | | | | | 4.99e−07 |
| cfDNA | damaged_HD_78_cfDNA | 100 μM_CuCl₂/ | Duplex- | 64248796 | 0.99 | 43911733 | 21 | 4.78E−07 | 3.13e−07- |
| | | H₂O_{2—}+_2 mU_DNase1 | Repair | | | | | | 7.31e−07 |
| cfDNA | damaged_HD_78_cfDNA | 100 μM_CuCl₂/ | Duplex- | 69169714 | 0.99 | 50237066 | 18 | 3.58E−07 | 2.27e−07- |
| | | H₂O_{2—}+_2 mU_DNase1 | Repair | | | | | | 5.66e−07 |
| cfDNA | HD_78 | NA | Duplex- | 112102088 | 0.98 | 220967435 | 20 | 9.05E−08 | 5.86e−08- |
| | | | Repair | | | | | | 1.40e−07 |
| cfDNA | HD_78 | NA | Duplex- | 121844386 | 0.98 | 205270556 | 24 | 1.17E−07 | 7.86e−08- |
| | | | Repair | | | | | | 1.74e−07 |
| cfDNA | HD_78 | NA | Duplex- | 115435308 | 0.98 | 246649702 | 24 | 9.73E−08 | 6.54e−08- |
| | | | Repair | | | | | | 1.45e−07 |
| cfDNA | 05055_33 | NA | Duplex- | 14814042 | 0.97 | 28090417 | 15 | 5.34E−07 | 3.24e−07- |
| | | | Repair | | | | | | 8.81e−07 |
| cfDNA | 05055_33 | NA | Conv. | 39619416 | 0.97 | 98148695 | 133 | 1.36E−06 | 1.14e−06- |
| | | | ER/AT | | | | | | 1.61e−06 |
| cfDNA | 05055_48 | NA | Duplex- | 39061056 | 0.99 | 63145388 | 27 | 4.28E−07 | 2.94e−07- |
| | | | Repair | | | | | | 6.22e−07 |
| cfDNA | 05055_48 | NA | Conv. | 61181344 | 0.98 | 112122125 | 426 | 3.80E−06 | 3.46e−06- |
| | | | ER/AT | | | | | | 4.18e−06 |
| cfDNA | 05055_73 | NA | Duplex- | 33936974 | 0.98 | 55844344 | 20 | 3.58E−07 | 2.32e−07- |
| | | | Repair | | | | | | 5.53e−07 |
| cfDNA | 05055_73 | NA | Conv. | 34623794 | 0.98 | 95264860 | 135 | 1.42E−06 | 1.20e−06- |
| | | | ER/AT | | | | | | 1.68e−06 |
| FFPE | 05055_106 | NA | Conv. | 53605918 | 0.99 | 20006860 | 521 | 2.60E−05 | 2.39e−05- |
| Tumor | | | ER/AT | | | | | | 2.84e−05 |
| Biopsy | | | | | | | | | |
| FFPE | 05055_106 | NA | Conv. | 66116050 | 0.99 | 23707349 | 665 | 2.81E−05 | 2.60e−05- |
| Tumor | | | ER/AT | | | | | | 3.03e−05 |
| Biopsy | | | | | | | | | |
| FFPE | 05055_106 | NA | Conv. | 95050922 | 0.99 | 43482673 | 1235 | 2.84E−05 | 2.69e−05- |
| Tumor | | | ER/AT | | | | | | 3.00e−05 |
| Biopsy | | | | | | | | | |
| FFPE | 05055_106 | NA | Duplex- | 67289076 | 0.99 | 49319461 | 467 | 9.47E−06 | 8.65e−06- |
| Tumor | | | Repair | | | | | | 1.04e−05 |
| Biopsy | | | | | | | | | |
| FFPE | 05055_106 | NA | Duplex- | 81280752 | 0.99 | 53118736 | 538 | 1.01E−05 | 9.31e−06- |
| Tumor | | | Repair | | | | | | 1.10e−05 |
| Biopsy | | | | | | | | | |
| FFPE | 05055_106 | NA | Duplex- | 68517836 | 0.99 | 50713014 | 551 | 1.09E−05 | 9.99e−06- |
| Tumor | | | Repair | | | | | | 1.18e−05 |
| Biopsy | | | | | | | | | |
| FFPE | 05055_95 | NA | Conv. | 85124228 | 0.99 | 17146216 | 879 | 5.13E−05 | 4.80e−05- |
| Tumor | | | ER/AT | | | | | | 5.48e−05 |
| Biopsy | | | | | | | | | |
| FFPE | 05055_95 | NA | Conv. | 76055416 | 0.99 | 24648513 | 1564 | 6.35E−05 | 6.04e−05- |
| Tumor | | | ER/AT | | | | | | 6.67e−05 |
| Biopsy | | | | | | | | | |
| FFPE | 05055_95 | NA | Conv. | 83267154 | 0.99 | 27917450 | 1734 | 6.21E−05 | 5.93e−05- |
| Tumor | | | ER/AT | | | | | | 6.51e−05 |
| Biopsy | | | | | | | | | |
| FFPE | 05055_95 | NA | Duplex- | 85086784 | 0.99 | 32413963 | 530 | 1.64E−05 | 1.50e−05- |
| Tumor | | | Repair | | | | | | 1.78e−05 |
| Biopsy | | | | | | | | | |
| FFPE | 05055_95 | NA | Duplex- | 74040728 | 0.99 | 28687673 | 471 | 1.64E−05 | 1.50e−05- |
| Tumor | | | Repair | | | | | | 1.80e−05 |
| Biopsy | | | | | | | | | |
| FFPE | 05055_95 | NA | Duplex- | 74421916 | 0.99 | 29219082 | 421 | 1.44E−05 | 1.31e−05- |
| Tumor | | | Repair | | | | | | 1.59e−05 |
| Biopsy | | | | | | | | | |
| FFPE | 05055_2 | NA | Conv. | 64724612 | 0.98 | 13700238 | 1078 | 7.87E−05 | 7.41e−05- |
| Tumor | | | ER/AT | | | | | | 8.35e−05 |
| Biopsy | | | | | | | | | |
| FFPE | 05055_2 | NA | Conv. | 52010368 | 0.99 | 23873009 | 2729 | 0.000114 | 1.10e−04- |
| Tumor | | | ER/AT | | | | | 31 | 1.19e−04 |
| Biopsy | | | | | | | | | |
| FFPE | 05055_2 | NA | Conv. | 78730640 | 0.99 | 24256287 | 2853 | 0.000117 | 1.13e−04- |
| Tumor | | | ER/AT | | | | | 62 | 1.22e−04 |
| Biopsy | | | | | | | | | |
| FFPE | 05055_2 | NA | Duplex- | 100214026 | 0.99 | 26565686 | 469 | 1.77E−05 | 1.61e−05- |
| Tumor | | | Repair | | | | | | 1.93e−05 |
| Biopsy | | | | | | | | | |
| FFPE | 05055_2 | NA | Duplex- | 103086654 | 0.99 | 27540728 | 457 | 1.66E−05 | 1.51e−05- |
| Tumor | | | Repair | | | | | | 1.82e−05 |
| Biopsy | | | | | | | | | |
| FFPE | 05055_2 | NA | Duplex- | 92651104 | 0.99 | 14224743 | 209 | 1.47E−05 | 1.28e−05- |
| Tumor | | | Repair | | | | | | 1.68e−05 |
| Biopsy | | | | | | | | | |
| FFPE | 05055_73 | NA | Conv. | 97662870 | 0.99 | 49482848 | 988 | 2.00E−05 | 1.88e−05- |
| Tumor | | | ER/AT | | | | | | 2.13e−05 |
| Biopsy | | | | | | | | | |
| FFPE | 05055_73 | NA | Conv. | 89638956 | 0.99 | 45128942 | 860 | 1.91E−05 | 1.78e−05- |
| Tumor | | | ER/AT | | | | | | 2.04e−05 |
| Biopsy | | | | | | | | | |
| FFPE | 05055_73 | NA | Conv. | 82714006 | 0.99 | 51361631 | 1184 | 2.31E−05 | 2.18e−05- |
| Tumor | | | ER/AT | | | | | | 2.44e−05 |
| Biopsy | | | | | | | | | |
| FFPE | 05055_73 | NA | Duplex- | 94834658 | 0.99 | 46828201 | 251 | 5.36E−06 | 4.74e−06- |
| Tumor | | | Repair | | | | | | 6.07e−06 |
| Biopsy | | | | | | | | | |
| FFPE | 05055_73 | NA | Duplex- | 99780400 | 0.99 | 43268486 | 197 | 4.55E−06 | 3.96e−06- |
| Tumor | | | Repair | | | | | | 5.23e−06 |
| Biopsy | | | | | | | | | |
| FFPE | 05055_73 | NA | Duplex- | 103370726 | 0.99 | 50748145 | 286 | 5.64E−06 | 5.02e−06- |
| Tumor | | | Repair | | | | | | 6.33e−06 |
| Biopsy | | | | | | | | | |
| FFPE | 05055_129 | NA | Conv. | 61850850 | 0.99 | 38198285 | 790 | 2.07E−05 | 1.93e−05- |
| Tumor | | | ER/AT | | | | | | 2.22e−05 |
| Biopsy | | | | | | | | | |
| FFPE | 05055_129 | NA | Conv. | 56961016 | 0.99 | 46259759 | 1411 | 3.05E−05 | 2.90e−05- |
| Tumor | | | ER/AT | | | | | | 3.21e−05 |
| Biopsy | | | | | | | | | |
| FFPE | 05055_129 | NA | Conv. | 83736366 | 0.99 | 67552550 | 2111 | 3.12E−05 | 2.99e−05- |
| Tumor | | | ER/AT | | | | | | 3.26e−05 |
| Biopsy | | | | | | | | | |
| FFPE | 05055_129 | NA | Duplex- | 63246978 | 0.99 | 46410054 | 300 | 6.46E−06 | 5.77e−06- |
| Tumor | | | Repair | | | | | | 7.24e−06 |
| Biopsy | | | | | | | | | |
| FFPE | 05055_129 | NA | Duplex- | 64728634 | 0.99 | 47452054 | 315 | 6.64E−06 | 5.94e−06- |
| Tumor | | | Repair | | | | | | 7.41e−06 |
| Biopsy | | | | | | | | | |
| FFPE | 05055_129 | NA | Duplex- | 65037294 | 0.99 | 67397721 | 463 | 6.87E−06 | 6.27e−06- |
| Tumor | | | Repair | | | | | | 7.52e−06 |
| Biopsy | | | | | | | | | |

At each concentration of CuCl₂/H₂O₂, it was found that the error rate increased with increasing amounts of DNase I, while the highest concentrations of both yielded an error rate 3.6-fold higher (C.I. 2.8-4.5) than that of untreated cfDNA. Expectedly, the largest increase in errors was observed which matched the expected C->A mutation signature of CuCl₂/H₂O₂ exposure (13.9-fold, FIG. 29) 3°. These results suggest that with conventional ER/AT, sequencing accuracy depends upon the extent of DNA damage in a sample.

To determine whether the impact of induced damage could be reverted, Duplex-Repair was applied to the most heavily damaged samples and sequenced them with the same gene panel. A significant reduction in error rate was observed, from 1.2e-6 to 3.7e-7, which was similar to the native cfDNA samples treated with conventional ER/AT (3.2e-7, FIG. 28A). Indeed, the impact of induced C->A errors was almost entirely ‘rescued’ (FIG. 29), while there was little change in error rates for other contexts (FIG. 29). Duplex-Repair was then applied to the native (i.e. undamaged) cfDNA and found the lowest error rates of all conditions tested (1.0e-7, FIG. 28A, FIG. 29). These results suggest that Duplex-Repair can revert the impact of induced DNA damage.

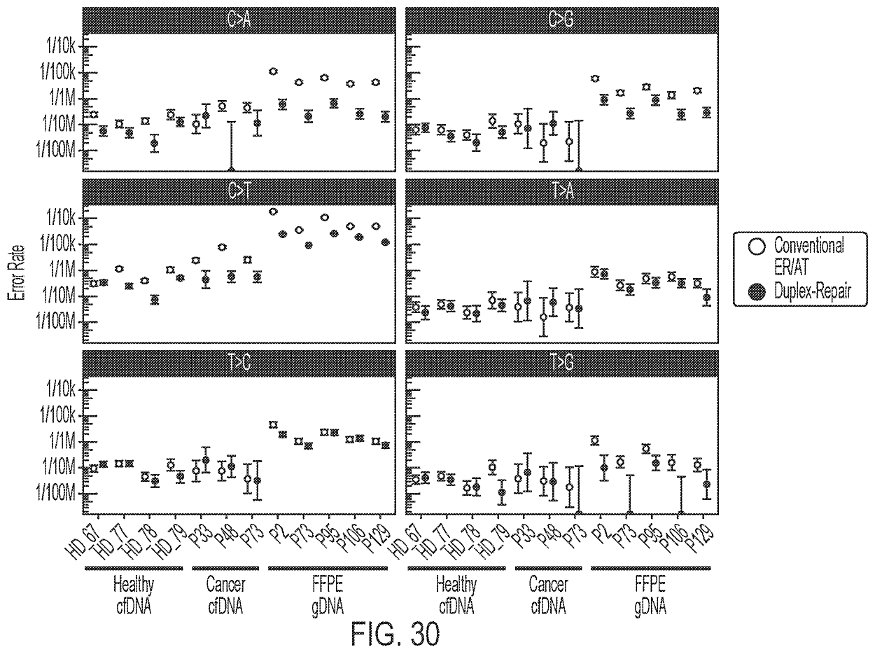

Then, it was sought to determine whether Duplex-Repair could provide higher accuracy than conventional ER/AT when used for duplex sequencing of clinical samples. A127-gene “pan-cancer” panel was applied across three sample types (FIG. 28B). In all samples, lower error rates were observed when Duplex-Repair was applied, in comparison to conventional ER/AT. In particular, the median error rates decreased from 5.8e-7 (range 3.2e-7-8.1e-7) to 3.0e-7 (range 9.2e-8-3.8e-7) for healthy cfDNA, from 1.4e-6 (range 1.4e-6-3.8e-6) to 4.3e-7 (range 3.6e-7-5.3e-7) for cancer cfDNA, and from 2.8e-5 (range 2.1e-5-1.1e-4) to 1.0e-5 (range 5.2e-6-1.7e-5) for FFPE tumor biopsies, which amounts to a median 2.5-fold (C.I. 1.6-3.3), 4.0-fold (C.I. 3.4-4.5), and 4.0-fold (C.I. 3.1-4.9) reduction in error rates respectively, with cancer patient cfDNA from P48 showing the largest 8.9-fold reduction in error rate (FIG. 28B). Furthermore, the most significant reductions in duplex sequencing error rates occurred for contexts of C->T (median 3.6-fold, C.I. 2.5-4.1 for healthy cfDNA; median 5.7-fold, C.I. 5.3-5.8 for cancer cfDNA; median 4.1-fold, C.I. 3.1-5.0 for FFPE biopsies), C->A (median 3.4-fold, C.I. 2.7-3.8 for healthy cfDNA; median 3.8-fold, C.I. 3.6-4.0 for cancer cfDNA; median 19.0-fold, C.I. 18.7-19.3 for FFPE biopsies), and C->G (median 1.9-fold, C.I. 1.2-2.5 for healthy cfDNA; median 1.5-fold, C.I. 1.0-1.9 for cancer cfDNA; median 6.2-fold, C.I. 5.8-6.6 for FFPE biopsies; FIG. 30, Table 3).

TABLE 3
Error rates and fold changes by mutation context for targeted panel sequencing. Duplex sequencing
error rates broken down by mutation context for three cancer patient cfDNA samples and five
FFPE tumor biopsies. The samples were treated with Duplex-Repair and conventional ER/AT.

| | | | Error Rate | Error Rate | | |
|---|---|---|---|---|---|---|
| | | Mutation | Conventional | Duplex- | Error Rate | |
| Specimen | Patient ID | Context | ER/AT | Repair | Fold Change | CI (95%) |
| cfDNA | 05055_33 | C > A | 1.07E−07 | 2.20E−07 | −2.06 | [−2.42, −1.71] |
| cfDNA | 05055_33 | C > G | 1.07E−07 | 7.34E−08 | 1.45 | [1, 1.9] |
| cfDNA | 05055_33 | C > T | 2.45E−06 | 4.40E−07 | 5.57 | [5.32, 5.81] |
| cfDNA | 05055_33 | T > A | 3.90E−08 | 6.92E−08 | −1.77 | [−2.03, −1.52] |
| cfDNA | 05055_33 | T > C | 7.81E−08 | 2.08E−07 | −2.66 | [−2.89, −2.43] |
| cfDNA | 05055_33 | T > G | 3.90E−08 | 6.92E−08 | −1.77 | [−2.03, −1.52] |
| cfDNA | 05055_48 | C > A | 5.25E−07 | 0 | inf | [0, 0] |
| cfDNA | 05055_48 | C > G | 2.02E−08 | 1.06E−07 | −5.25 | [−5.28, −5.23] |
| cfDNA | 05055_48 | C > T | 7.89E−06 | 6.01E−07 | 13.13 | [12.85, 13.42] |
| cfDNA | 05055_48 | T > A | 1.60E−08 | 5.74E−08 | −3.59 | [−3.64, −3.54] |
| cfDNA | 05055_48 | T > C | 7.99E−08 | 1.15E−07 | −1.44 | [−2.06, −0.82] |
| cfDNA | 05055_48 | T > G | 3.20E−08 | 2.87E−08 | 1.11 | [0.34, 1.89] |
| cfDNA | 05055_73 | C > A | 4.39E−07 | 1.17E−07 | 3.77 | [3.57, 3.96] |
| cfDNA | 05055_73 | C > G | 2.31E−08 | 0 | inf | [0, 0] |
| cfDNA | 05055_73 | C > T | 2.54E−06 | 5.83E−07 | 4.36 | [3.91, 4.81] |
| cfDNA | 05055_73 | T > A | 3.85E−08 | 3.32E−08 | 1.16 | [0.46, 1.86] |
| cfDNA | 05055_73 | T > C | 3.85E−08 | 3.32E−08 | 1.16 | [0.46, 1.86] |
| cfDNA | 05055_73 | T > G | 1.92E−08 | 0 | inf | [0, 0] |
| FFPE Tumor Biopsy | 05055_106 | C > A | 3.80E−06 | 2.59E−07 | 14.68 | [14.41, 14.95] |
| FFPE Tumor Biopsy | 05055_106 | C > G | 1.43E−06 | 2.59E−07 | 5.54 | [5.14, 5.94] |
| FFPE Tumor Biopsy | 05055_106 | C > T | 5.33E−05 | 1.98E−05 | 2.70 | [1.77, 3.63] |
| FFPE Tumor Biopsy | 05055_106 | T > A | 5.48E−07 | 3.35E−07 | 1.64 | [0.87, 2.4] |
| FFPE Tumor Biopsy | 05055_106 | T > C | 1.24E−06 | 1.41E−06 | −1.14 | [−2.1, −0.17] |
| FFPE Tumor Biopsy | 05055_106 | T > G | 1.69E−07 | 0 | inf | [0, 0] |
| FFPE Tumor Biopsy | 05055_129 | C > A | 4.36E−06 | 2.07E−07 | 21.09 | [20.87, 21.3] |
| FFPE Tumor Biopsy | 05055_129 | C > G | 2.13E−06 | 2.84E−07 | 7.47 | [7.07, 7.88] |
| FFPE Tumor Biopsy | 05055_129 | C > T | 5.17E−05 | 1.25E−05 | 4.14 | [3.24, 5.05] |
| FFPE Tumor Biopsy | 05055_129 | T > A | 3.25E−07 | 9.53E−08 | 3.41 | [3.03, 3.79] |
| FFPE Tumor Biopsy | 05055_129 | T > C | 1.11E−06 | 7.87E−07 | 1.41 | [0.52, 2.31] |
| FFPE Tumor Biopsy | 05055_129 | T > G | 1.37E−07 | 2.38E−08 | 5.77 | [5.7, 5.84] |
| FFPE Tumor Biopsy | 05055_2 | C > A | 1.16E−05 | 6.10E−07 | 19.02 | [18.72, 19.31] |
| FFPE Tumor Biopsy | 05055_2 | C > G | 6.14E−06 | 9.41E−07 | 6.53 | [6.01, 7.04] |
| FFPE Tumor Biopsy | 05055_2 | C > T | 1.89E−04 | 2.53E−05 | 7.50 | [6.63, 8.38] |
| FFPE Tumor Biopsy | 05055_2 | T > A | 9.43E−07 | 7.24E−07 | 1.30 | [0.44, 2.17] |
| FFPE Tumor Biopsy | 05055_2 | T > C | 4.91E−06 | 1.96E−06 | 2.50 | [1.74, 3.26] |
| FFPE Tumor Biopsy | 05055_2 | T > G | 1.17E−06 | 1.03E−07 | 11.33 | [11.27, 11.39] |
| FFPE Tumor Biopsy | 05055_73 | C > A | 4.47E−06 | 2.13E−07 | 21.01 | [20.81, 21.2] |
| FFPE Tumor Biopsy | 05055_73 | C > G | 1.71E−06 | 2.74E−07 | 6.24 | [5.84, 6.65] |
| FFPE Tumor Biopsy | 05055_73 | C > T | 3.71E−05 | 9.63E−06 | 3.85 | [2.96, 4.74] |
| FFPE Tumor Biopsy | 05055_73 | T > A | 2.67E−07 | 1.86E−07 | 1.43 | [0.65, 2.22] |
| FFPE Tumor Biopsy | 05055_73 | T > C | 1.08E−06 | 7.32E−07 | 1.47 | [0.6, 2.35] |
| FFPE Tumor Biopsy | 05055_73 | T > G | 1.78E−07 | 0 | inf | [0, 0] |
| FFPE Tumor Biopsy | 05055_95 | C > A | 6.60E−06 | 6.79E−07 | 9.73 | [9.31, 10.15] |
| FFPE Tumor Biopsy | 05055_95 | C > G | 2.91E−06 | 9.28E−07 | 3.14 | [2.48, 3.8] |
| FFPE Tumor Biopsy | 05055_95 | C > T | 1.12E−04 | 2.77E−05 | 4.05 | [3.14, 4.97] |
| FFPE Tumor Biopsy | 05055_95 | T > A | 4.67E−07 | 3.69E−07 | 1.27 | [0.41, 2.12] |
| FFPE Tumor Biopsy | 05055_95 | T > C | 2.42E−06 | 2.28E−06 | 1.06 | [0.08, 2.05] |
| FFPE Tumor Biopsy | 05055_95 | T > G | 5.77E−07 | 1.52E−07 | 3.80 | [3.48, 4.12] |

Notably, it was observed that base errors were more significantly enriched at the ends of fragments with 34% of a total of 9,122 base errors (after normalizing for total bases evaluated) being in the first base from either duplex fragment end for Duplex-Repair as compared to only 15% of a total of 31,100 base errors for conventional ER/AT (FIG. 28C, FIG. 17). Overall, it was estimated that 74% of base errors were concentrated within 12 bp from the end of the fragment for Duplex-Repair, in contrast with 68% for conventional ER/AT. It is worth noting that these base errors can be removed in-silico by filtering regions less than 12 bp from the duplex fragment ends. Finally, the relationship between strand resynthesis fractions and observed error rates was examined across clinical samples. A strong overall correlation was observed between the fractions of interior base pairs resynthesized and the error rates of duplex sequencing (Pearson's r=0.859; FIG. 28D). These results establish that Duplex-Repair could afford consistently higher accuracy for duplex sequencing of clinical samples by limiting resynthesis during library construction.

It is shown herein that existing ‘End Repair/dA-tailing’ (ER/AT) methods could resynthesize large portions of each DNA duplex, particularly when there are interior nicks, gaps, or long 5′ overhangs. This is a major problem for techniques such as duplex sequencing which require a consensus of reads from both strands. Presented herein is a solution called Duplex-Repair which conducts ER/AT in a careful, stepwise manner. It is shown that it limits resynthesis by 8- to 464-fold, reverts the impact of induced DNA damage, and confers up to 8.9-fold higher accuracy in duplex sequencing of a cancer gene panel for specimens such as cfDNA and FFPE tumor biopsies. Considering the widespread use of duplex sequencing in biomedical research and diagnostic testing, these findings are likely to have broad impact in many areas such as oncology, infectious diseases, immunology, prenatal medicine, forensics, genetic engineering, and beyond.

This Example has characterized this major Achilles' heel in ER/AT and provided a solution to restore highly-accurate DNA sequencing despite DNA damage. While it has been recognized that false mutations accumulate at fragment ends in duplex sequencing data due to the fill-in of short 5′ overhangs, the extent to which false mutations could manifest within the interior of each DNA duplex as a result of ER/AT has not been established. The single-molecule sequencing assay described herein, has provided novel insight into ER/AT and mechanisms of DNA repair. Indeed, it was astonishing to find that 7-9%, 15-17%, and 32-57% of base pairs>12 bp from the ends of each duplex in healthy cfDNA, cancer patient cfDNA, and FFPE tumor biopsies, respectively, could be resynthesized when conventional ER/AT methods were applied. Further, the induction of varied base and backbone damage has shown how the two together create the ‘perfect storm’ for errors when conventional ER/AT methods are applied. The observation presented herein that both strand resynthesis and error rate increase with DNase I concentration suggests that the reliability of diagnostic tests such as liquid biopsies could be affected by the nuclease activity in an individual's bloodstream. Given the wide variation in quality of clinical specimens, these findings have important implications for the field.

As shown herein, ER/AT methods function like a ‘pencil and eraser,’ rewriting the nucleobases downstream of discontinuities in the phosphodiester backbone, and spurring false detection of lesions or alterations originally confined to one strand. Meanwhile, the solution of Duplex-Repair offers one of the first known approaches to preserve the sequence integrity of duplex DNA and thus, improve the reliability of methods which leverage the duplicity of genetic information in DNA.

### References in Example 2

Each of the Following References are Hereby Incorporated in their Entireties
1. Ellegren, H. & Galtier, N. Determinants of genetic diversity. Nature Reviews Genetics vol. 17 422-433 (2016).
2. Smith, M. J. et al. Loss-of-function mutations in SMARCE1 cause an inherited disorder of multiple spinal meningiomas. Nat. Genet. 45, 295-298 (2013).
3. Zahn, L. M. Mapping genotype to phenotype. Science vol. 362 555.4-556 (2018).
4. Ludwig, L. S. et al. Lineage Tracing in Humans Enabled by Mitochondrial Mutations and Single-Cell Genomics. Cell 176, 1325-1339.e22 (2019).
5. Salk, J. J. & Horwitz, M. S. Passenger mutations as a marker of clonal cell lineages in emerging neoplasia. Semin. Cancer Biol. 20, 294-303 (2010).
6. MacArthur, D. G. et al. Guidelines for investigating causality of sequence variants in human disease. Nature 508, 469-476 (2014).
7. Ashley, E. A. Towards precision medicine. Nat. Rev. Genet. 17, 507-522 (2016).
8. Gerlinger, M. et al. Intratumor heterogeneity and branched evolution revealed by multiregion sequencing. N. Engl. J. Med. 366, 883-892 (2012).
9. Vasan, N., Baselga, J. & Hyman, D. M. A view on drug resistance in cancer. Nature vol. 575 299-309 (2019).
10. Zahn, L. M. Somatic mosaicism in normal tissues. Science vol. 364 966.10-968 (2019).
11. Genovese, G. et al. Clonal hematopoiesis and blood-cancer risk inferred from blood DNA sequence. N. Engl. J. Med. 371, 2477-2487 (2014).
12. Anzalone, A. V., Koblan, L. W. & Liu, D. R. Genome editing with CRISPR-Cas nucleases, base editors, transposases and prime editors. Nature Biotechnology vol. 38 824-844 (2020).
13. Matsumura, S., Fujita, Y., Yamane, M., Morita, O. & Honda, H. A genome-wide mutation analysis method enabling high-throughput identification of chemical mutagen signatures. Sci. Rep. 8, 9583 (2018).
14. D'Gama, A. M. & Walsh, C. A. Somatic mosaicism and neurodevelopmental disease. Nat. Neurosci. 21, 1504-1514 (2018).
15. Bell, A. D. et al. Insights into variation in meiosis from 31,228 human sperm genomes. Nature 583, 259-264 (2020).
16. Blauwkamp, T. A. et al. Analytical and clinical validation of a microbial cell-free DNA sequencing test for infectious disease. Nat Microbiol 4, 663-674 (2019).
17. Lennon, A. M. et al. Feasibility of blood testing combined with PET-CT to screen for cancer and guide intervention. Science 369, (2020).
18. Lanman, R. B. et al. Analytical and Clinical Validation of a Digital Sequencing Panel for Quantitative, Highly Accurate Evaluation of Cell-Free Circulating Tumor DNA. PLoS One 10, e0140712 (2015).
19. Chen, L., Liu, P., Evans, T. C., Jr & Ettwiller, L. M. DNA damage is a pervasive cause of sequencing errors, directly confounding variant identification. Science 355, 752-756 (2017).
20. Costello, M. et al. Discovery and characterization of artifactual mutations in deep coverage targeted capture sequencing data due to oxidative DNA damage during sample preparation. Nucleic Acids Res. 41, e67 (2013).
21. Wong, S. Q. et al. Sequence artefacts in a prospective series of formalin-fixed tumours tested for mutations in hotspot regions by massively parallel sequencing. BMC Med. Genomics 7, 23 (2014).
22. Salk, J. J., Schmitt, M. W. & Loeb, L. A. Enhancing the accuracy of next-generation sequencing for detecting rare and subclonal mutations. Nat. Rev. Genet. 19, 269-285 (2018).
23. Schmitt, M. W. et al. Detection of ultra-rare mutations by next-generation sequencing. Proc. Natl. Acad. Sci. U.S.A. 109, 14508-14513 (2012).
24. Parsons, H. A. et al. Sensitive Detection of Minimal Residual Disease in Patients Treated for Early-Stage Breast Cancer. Clin. Cancer Res. 26, 2556-2564 (2020).
25. Zhang, A. et al. Solid-phase enzyme catalysis of DNA end repair and 3′ A-tailing reduces GC-bias in next-generation sequencing of human genomic DNA. Sci. Rep. 8, 15887 (2018).
26. Jiang, P. et al. Detection and characterization of jagged ends of double-stranded DNA in plasma. Genome Res. 30, 1144-1153 (2020).
27. Adalsteins son, V. A. et al. Scalable whole-exome sequencing of cell-free DNA reveals high concordance with metastatic tumors. Nat. Commun. 8, 1324 (2017).
28. Zatopek, K. M. et al. RADAR-seq: A RAre DAmage and Repair sequencing method for detecting DNA damage on a genome-wide scale. DNA Repair 80, 36-44 (2019).
29. Xiao, C.-L. et al. N6-Methyladenine DNA Modification in the Human Genome. Mol. Cell 71, 306-318.e7 (2018).
30. Lee, D.-H. Oxidative DNA damage induced by copper and hydrogen peroxide promotes CG->TT tandem mutations at methylated CpG dinucleotides in nucleotide excision repair-deficient cells. Nucleic Acids Research vol. 30 3566-3573 (2002).
31. Abascal, F. et al. Somatic mutation landscapes at single-molecule resolution. Nature (2021) doi:10.1038/s41586-021-03477-4.

### Example 3: Duplex-Repair Maximizes the ‘Original’ Nucleobase Composition of Each DNA Duplex

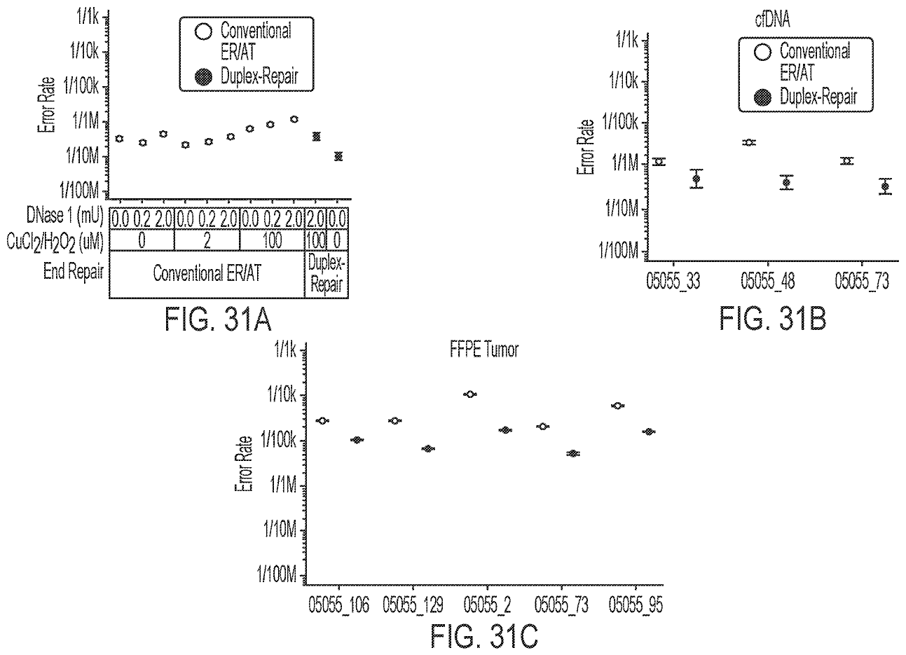

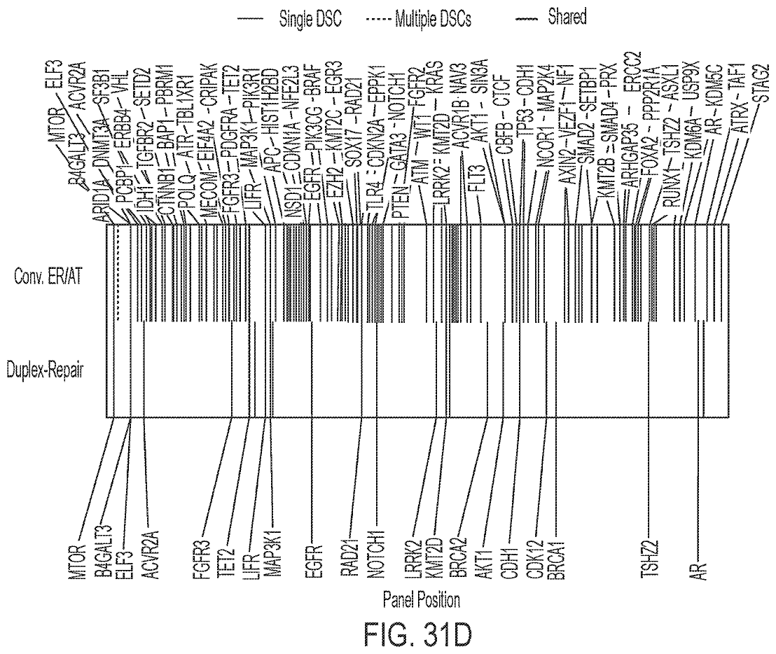

In connection with FIG. 13A-13C and FIG. 31A-31D, the following conclusions are made:

It is first confirmed that conventional ER/AT methods could substantially resynthesize each duplex, where there are nicks, gaps, and long overhangs (FIG. 13A).

It was discovered that >7-52% of base pairs in cfDNA & FFPE tumor biopsies could be resynthesized (FIG. 13B).

It is confirmed there is 5.5-7.5-fold less resynthesis (FIG. 13C) with Duplex-Repair (e.g. FIG. 1 and described herein).

It was found that Duplex-Repair rescues the impact of induced DNA damage (FIGS. 31A-31B) and confers higher accuracy in duplex sequencing (FIGS. 31C-31D).

### Example 4: Additional Methods of Duplex-Repair

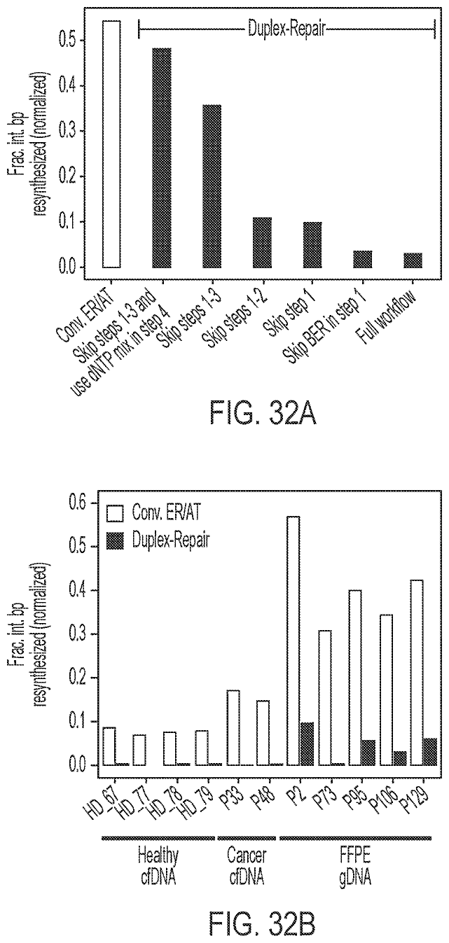

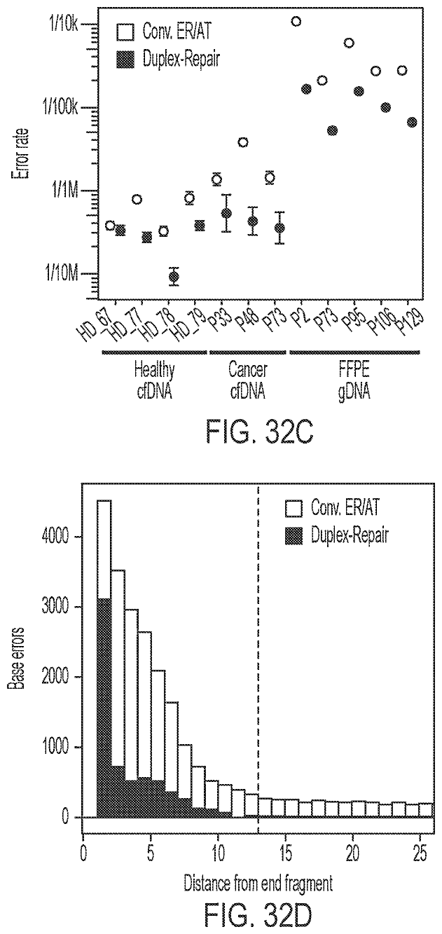

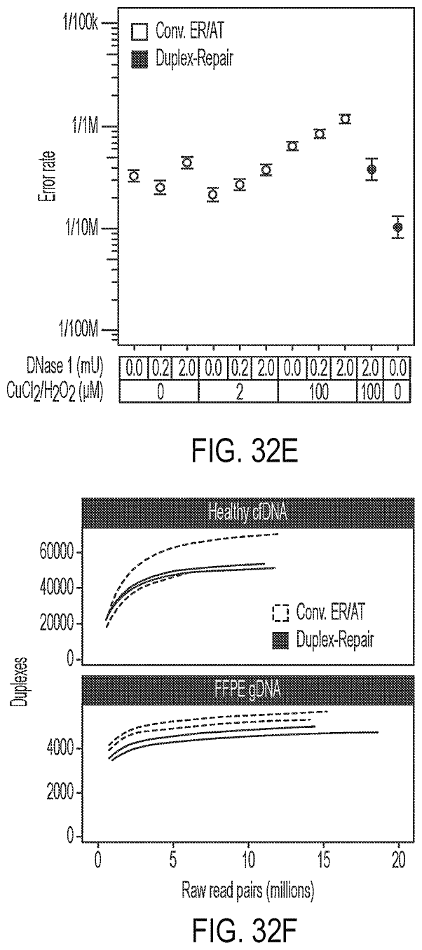

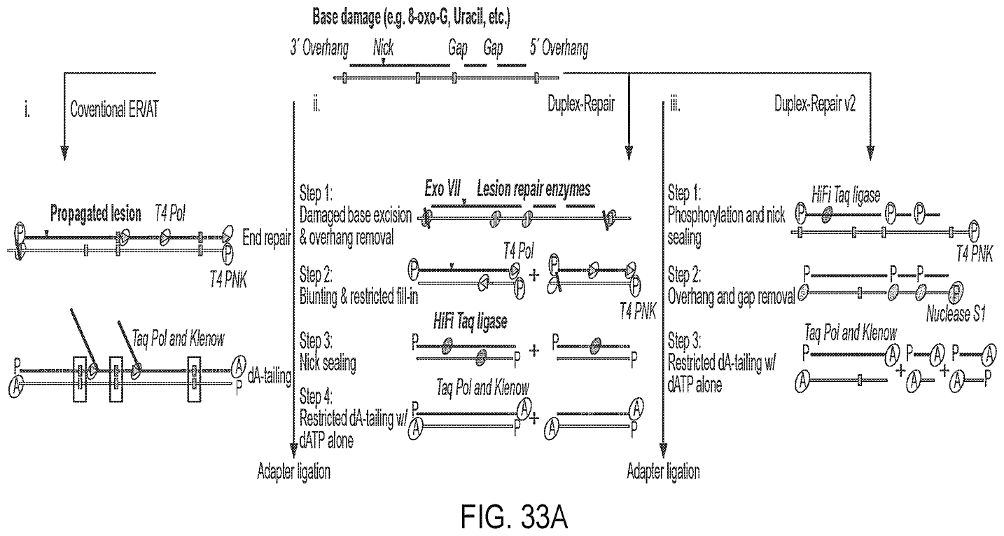

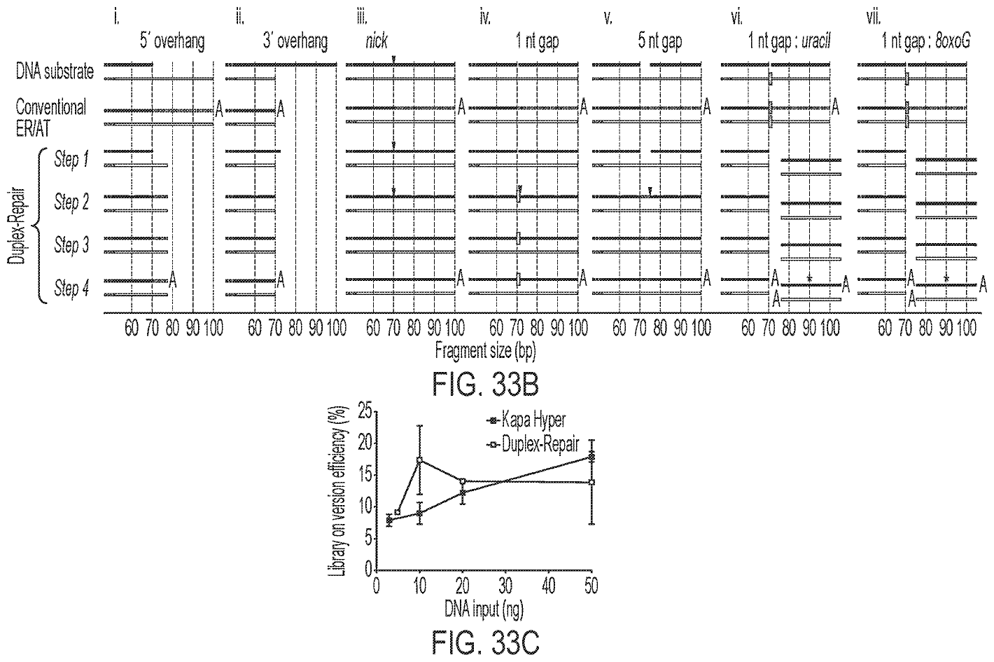

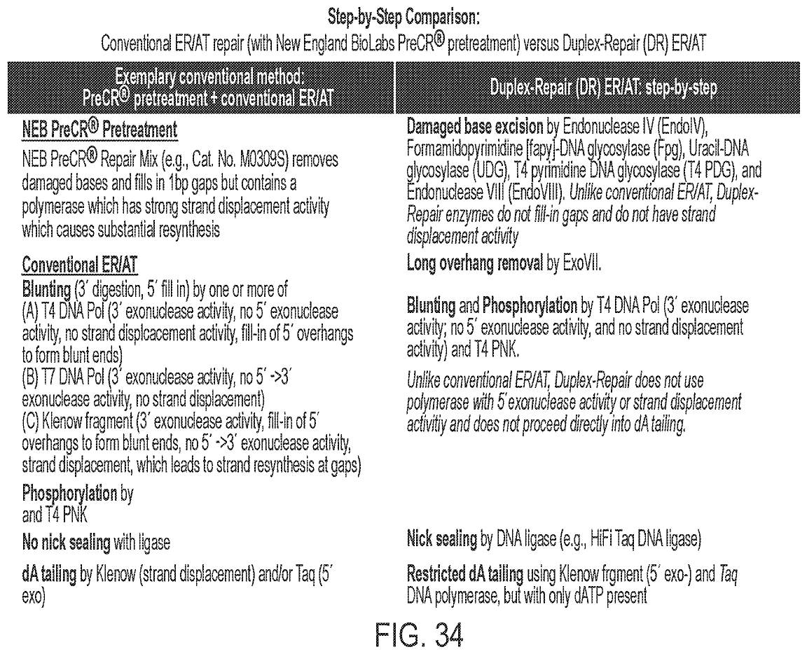

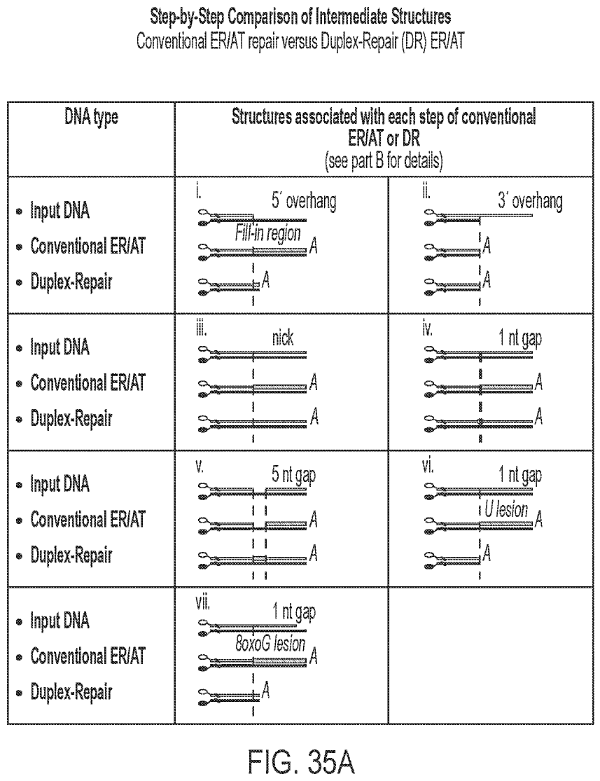

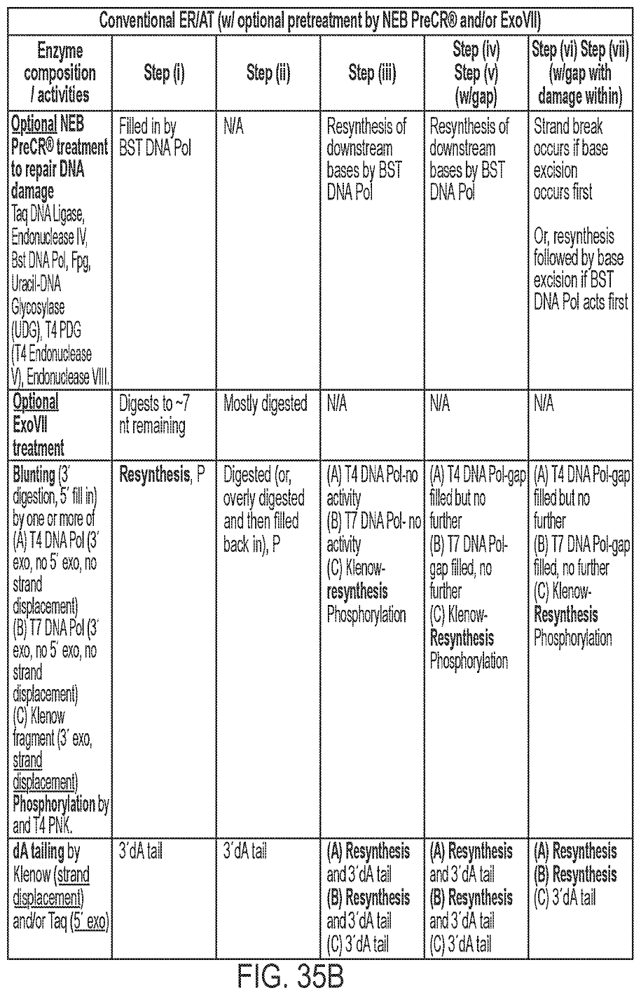

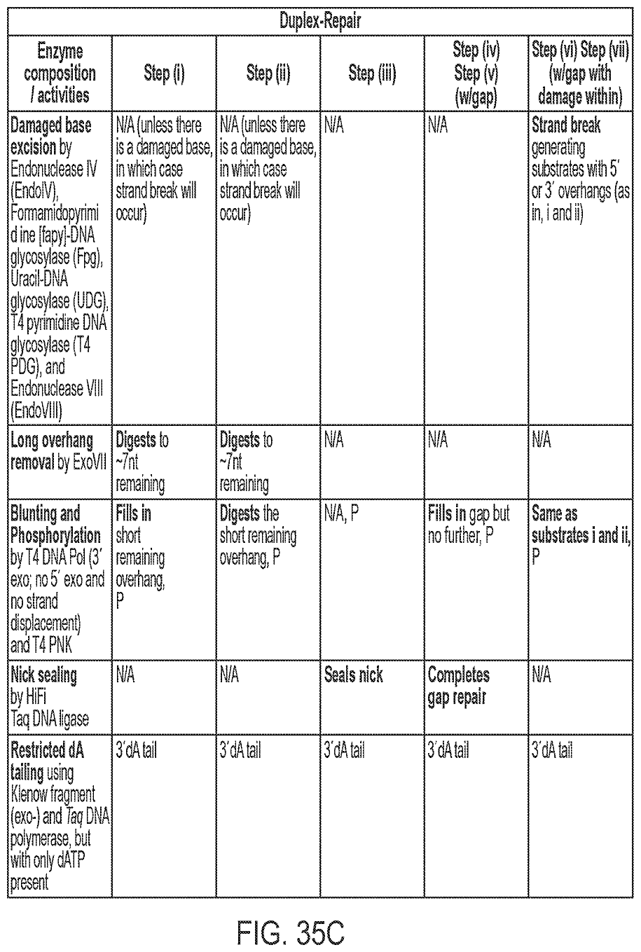

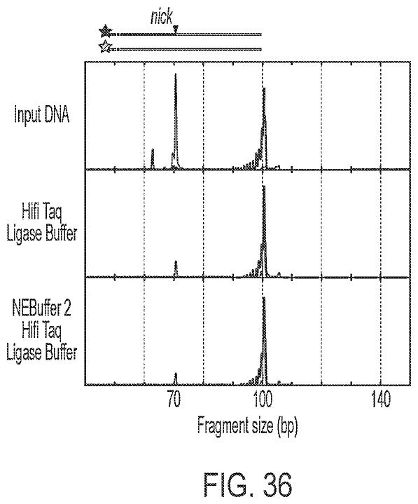

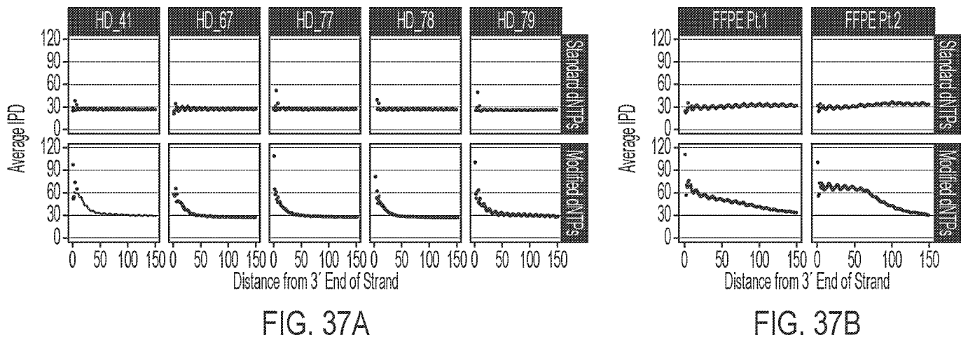

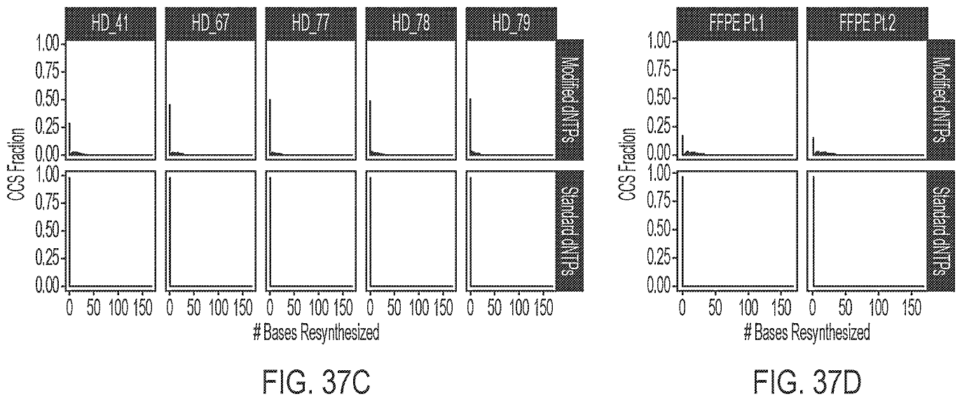

Duplex-Repair as described in the Examples above still requires restricted fill-in of gaps and short overhangs remaining after ExoVII treatment (FIGS. 32A-32B), which leaves a theoretical ‘non-zero’ potential for base damage errors to be copied to both strands (FIGS. 32C-32F). An objective, therefore, is to create the next generation of Duplex-Repair, e.g., ‘v2’ which fully eliminates the need for strand resynthesis and thus, in theory, leaves ‘zero’ potential for error propagation to both strands, while retaining high molecular recovery. The proposal to accomplish this is detailed in FIG. 33A and involves using Nuclease 51, a single-strand endonuclease demonstrated to cleave single-stranded gap regions and overhangs in duplex DNA and produce fully blunted duplexes, while leaving double-stranded regions intact. Through this functionality, Duplex-Repair v2 improves on its predecessor by eliminating the need to excise damaged bases, to treat with ExoVII, or to fill gaps and short 5′ overhangs which were left after ExoVII treatment.

Duplex Repair v2 consists of three steps: (1) phosphorylation and nick sealing; (2) overhang and gap removal; and (3) restricted dA-tailing FIG. 33A. In Step 1, T4 polynucleotide kinase and HiFi Taq Ligase are used to ensure that DNA has 5′ phosphate and 3′ hydroxyl moieties and that nicks are sealed, respectively. In Step 2, Nuclease 51 removes 5′ and 3′ overhangs while also digesting gap regions as small as one nucleotide in length into soluble dNMPs (e.g., deoxy-nucleoside monophosphates), producing blunted duplexes at the previous edges of these motifs. Of note, 5′ phosphate and 3′ hydroxyl moieties are left after Nuclease S1 digestion. In Step 3, Klenow fragment (exo-) and Taq DNA polymerase are supplemented with dATP only (i.e., dCTP, dGTP, and/or dTTP are not provided) for restricted dA-tailing as may be utilized in the earlier Duplex-Repair method. This ensures that only a 3′ deoxyadenosine tail can be added.

Capillary electrophoresis (FIG. 33B), ddPCR (FIG. 33C), single-molecule sequencing (FIG. 32A), and duplex sequencing (FIG. 32C) assays will be used to characterize Duplex-Repair v2, its molecular recovery, the number of bases resynthesized, and the duplex sequencing error rates, respectively, in comparison to commercial ER/AT kits. Each step will be tested on its own using fluorescently-labelled synthetic oligonucleotides bearing nicks, gaps, and overhangs for evaluation with capillary electrophoresis (CE), to gauge enzymatic activity and conversion efficiency qualitatively, initially from CE traces (FIG. 33B). With the intended performance confirmed, Duplex-Repair v2 will be formulated into a method of the fewest possible steps, eliminating buffer exchanges and optimizing buffer compositions and experimental conditions (e.g., time, temperature, concentration, and alternative enzymes), aiming to maximize molecular recovery. Varied inputs (e.g., <1-1000 ng) of buffy-coat-derived genomic DNA will then be tested, from a healthy donor whose germline sequence has been determined, sheared to different median insert sizes (e.g., 50-250 bp) with different methods (e.g., sonication, enzymatic digestion). The number of bases resynthesized, the duplex sequencing error rates, and the molecular recoveries of Duplex-Repair v2 will then be characterized vs. commercial ER/AT using the KAPA™ HyperPrep and NEB™ Ultra II kits. Challenging samples such as DNA subjected to varied extents of base and backbone damage (FIG. 32E) and formalin fixed tumor biopsies (FIGS. 32B-32C) will also be tested to explore the impact of sample handling conditions, such as varied freeze-thaw cycles, buffer compositions, and extended room temperature storage. Duplex-Repair v2 will further improve upon the performance of Duplex-Repair, reducing the number of bases resynthesized to zero (FIG. 32B) for most samples including FFPE tumor biopsies, and thus maximizing duplex sequencing accuracy (FIG. 32C). This could also limit the need to trim the last 12 bp of each fragment, as currently required with Duplex Sequencing methods, and improve data output. Future strategies for DNA fragmentation could limit the need for ER/AT if, for instance, overhang length could be limited, or adapters directly added via tagmentation or ligation at double-stranded breaks. However, sample types which are naturally fragmented such as cfDNA will likely always require ER/AT.

### Sequences

The present disclosure references a number of different enzymes that may be used in the presently disclosed methods. Such enzymes are well-known in the art and can be obtained from any suitable source, including commercial sources, such as New England BioLabs, AMSBIO, and Sigma-Aldrich. A person having ordinary skill in the art will understand based on the name of the enzymes disclosed herein the identity of the enzymes disclosed herein and how to obtain said enzymes without undue experimentation. While not intending to limit the present disclosure in any way, the following are examples of enzyme amino acid sequences that may be used in the presently disclosed methods. The disclosure contemplates the use of any of the below amino acid sequences, or amino acid sequences having at least 75%, or at least 80%, or at least 85%, or at least 90%, or at least 95%, or at least 99%, or up to 100% sequence identity with any of the herein disclosed amino acid sequences.

| Name of enzyme | Exemplary Amino Acid Sequences |
|---|---|
| endonuclease IV (EndoIV) | GENBANK NO: |
| Endonuclease IV (EndoIV) or | WP_000873890 |
| deoxyribonuclease IV cleaves | MKYIGAHVSAAGGLANAAIRAAEIDATAFALFTKNQRQ |
| phosphodiester bonds at | WRAAPLTTQTIDEFKAACEKYHYTSAQILPHDSYLINLG |
| apurinic or apyrimidinic sites | HPVTEALEKSRDAFIDEMQRCEQLGLSLLNFHPGSHLM |
| (AP sites) to produce new 5′- | QISEEDCLARIAESINIALDKTQGVTAVIENTAGQGSNLG |
| ends that are base-free | FKFEHLAAIIDGVEDKSRVGVCIDTCHAFAAGYDLRTPA |
| deoxyribose 5-phosphate | ECEKTFADFARIVGFKYLRGMHLNDAKSTFGSRVDRHH |
| residues | SLGEGNIGHDAFRWIMQDDRFDGIPLILETINPDIWAEEI |
| | AWLKAQQTEKAVA (SEQ ID NO: 3) |
| formamidopyrimidine [fapy]- | GENBANK NO: |
| DNA glycosylase (Fpg) | ADX48752 |
| Formamidopyrimidine-DNA | MPELPEVETSRRGIEPHLVGATILHAVVRNGRLRWPVSE |
| glycosylase is a DNA | EIYRLSDQPVLSVQRRAKYLLLELPEGWIIIHLGMSGSLR |
| glycosylase that releases | ILPEELPPEKHDHVELVMSNGKVLRYTDPRRFGAWLWT |
| damaged bases preferentially | KELEGHNVLTHLGPEPLSDDFNGEYLHQKCAKKKTAIK |
| from duplex DNA. It has an | PWLMDNKLVVGVGNIYASESLFAAGIHPDRLASSLSLAE |
| associated class I AP | CELLARVIKAVLLRSIEQGGTTLKDFLQSDGKPGYFAQE |
| (apurinic/apyrimidinic) lyase | LQVYGRKGEPCRVCGTPIVATKHAQRATFYCRQCQK |
| activity. Fpg Cleaves the DNA | (SEQ ID NO: 4) |
| backbone to generate a single- | |
| strand break at the site of the | |
| removed base, The C-O-P | |
| bond 3′ to the apurinic or | |
| apyrimidinic site in DNA is | |
| broken by a beta-elimination | |
| reaction, leaving 3′ and 5′ | |
| phosphoryl groups. | |
| uracil-DNA glycosylase | GENBANK NO: |
| (UDG) | WP_001262716.1 |
| Uracil-DNA glycosylase | MANELTWHDVLAEEKQQPYFLNTLQTVASERQSGVTIY |
| excises uracil residues from | PPQKDVFNAFRFTELGDVKVVILGQDPYHGPGQAHGLA |
| the DNA which can arise as a | FSVRPGIAIPPSLLNMYKELENTIPGFTRPNHGYLESWAR |
| result of misincorporation of | QGVLLLNTVLTVRAGQAHSHASLGWETFTDKVISLINQ |
| dUMP residues by DNA | HREGVVFLLWGSHAQKKGAIIDKQRHHVLKAPHPSPLS |
| polymerase or due to | AHRGFFGCNHFVLANQWLEQRGETPIDWMPVLPAESE |
| deamination of cytosine. | (SEQ ID NO: 5) |
| | GENBANK NO: |
| | XP_009306684 |
| | MVQRTLLDFVRKKNTSPTCVKVDVDEESSEEVMRQSLK |
| | RTAEDMEALPPPVKKKMELDACLSSLIIDPDWRAFLHPL |
| | TSAPSFQRVCQFVEMEAASGKVILPPRELIFSAFNSTPLE |
| | RVKVVLLGQDPYHNIGQAHGLCFSVRPGMRPPPSLVNM |
| | YKELSADIPGFKAPSHGYLQHWAEQGVLMLNATLTVEA |
| | HKANSHATCGWQAFTDGVIHLLSDAHKKPIVFLLWGGF |
| | ARKKITLIDRKRHVVIENAHPSPLSATKWWGSRPFSKCN |
| | DALTKIGHTPIDWSLPMTVIDWSLPMTV (SEQ ID NO: 6) |
| | GENBANK NO: |
| | ADV66263.1 |
| | MTQPDLFNPGPALPDLPPSWAAVLVDELHSPRFRALMD |
| | FVVQERAEHAVYPPEADVFNALRLTPLEDVKVLILGQD |
| | PYHGAGQAHGLAFSVQPGVRVPPSLKNIYKELQADVGF |
| | VPPKHGHLTAWARRGVLLLNAVLTVRAGEPNSHAGRG |
| | WEHVTDAVIRALNAREERVVFVLWGAYARKKAKLITA |
| | PQHVIIESAHPSPLSVAKFLGTKPFSQVNAALDEAGRGAI |
| | DWQLPADPNEQ (SEQ ID NO: 7) |
| endonuclease VIII (Endo VIII) | GENBANK NO: |
| endonuclease VIII is a DNA- | BAA20414.1 |
| (apurinic or apyrimidinic site) | MPEGPEIRRAADNLEAAIKGKPLTDVWFAFPQLKPYQS |
| lyase involved in base excision | QLIGQHVTHVETRGKALLTHESNDLTLYSHNQLYGVWR |
| repair of DNA damaged by | VVDTGEEPQTTRVLRVKLQTADKTILLYSASDIEMLTPE |
| oxidation or by mutagenic | QLTTHPFLQRVGPDVLDPNLTPEVVKERLLSPRFRNRQF |
| agents; acts as DNA | AGLLLDQAFLAGLGNYLRVEILWQVGLTGNHKAKDLN |
| glycosylase that recognizes | AAQLDALAHALLEIPRFSYATRGQVDENKHHGALFRFK |
| and removes damaged bases | VFHRDGEPCERCGSIIEKTTLSSRPFYWCPGCQH (SEQ ID |
| with a preference for oxidized | NO: 8) |
| pyrimidines | GENBANK NO: |
| | WP_002894935.1 |
| | MPEGPEIRRAADKLEAAIKGEPLTNVWFAFPQLQPYQTQ |
| | LIGQRVTHIATRGKALLTHFSGGLTLYSHNQLYGVWRV |
| | VDAGVEPQSNRVLRVRLQTASKAILLYSASDIDILTAEQ |
| | VANHPFLLRVGPDVLDMTLTAEQVKARLLSAKFRNRQF |
| | SGLLLDQAFLAGLGNYLRVEILWQVGLSGKRKAAELSD |
| | SQLDALAHALLDIPRLSYRTRGLVDDNKHHGALFRFKV |
| | FHRDGERCERCGGIIEKTTLSSRPFYWCPGCQH (SEQ ID |
| | NO: 9) |
| polynucleotide kinase | GENBANK NO: |
| | VAX48620.1 |
| | MIWQLTDDKRWSALRQRFSWVEEMHHTPQDPEHHGEG |
| | DVGVHTEMVLNALITLPEFQQLPAQQQEVLWAAALLH |
| | DVEKRSTTVQENGRIQSPGHARRGELTARQILWRDIPTP |
| | FVLREQIVALVRLHGLPLWLLERPEPERLLLTAAMRIDT |
| | RLLALLARADLLGRQSPDQQSMLERIDLFELFCHEQQC |
| | WGKMRPFVSDSARWHYLTHEQSSPDFVPWEAEPFEVIL |
| | LCGLPGMGKDRYISEQCQGMDVISLDDMRRRINASPDD |
| | KTATGRIVQQAKEEARVFLRQKKPFIWNATNITRQLRSQ |
| | LISLFTAYGARVKIVYLEVPWAQWNQQNARREYAVPEA |
| | VVMRMASRLEVPQPDEAHSVEYRMTDR (SEQ ID NO: |
| | 10) |
| DNA ligase | GENBANK NO: |
| ATP-dependent DNA ligase | WP_072770587.1 |
| catalyzes the formation of a | MKKLIALKHKLDEMKAMGTNAKKEALANMDDFEQSM |
| phosphodiester at the site of a | VSLMLNPFIRFGVKKYKVAEPLSESVPSDEKAIDILNKLA |
| single-strand break in duplex | SRELTGNAAIAAVESIVASMCADGQDVFRRFLLKDPKA |
| DNA | GVGISLCNKVFENPIPKFEVQLASPYKEKGDKYPFKPNP |
| | KAKWPMIGSLKLDGLRVICEVIVDEEEVNFLSRTGNPITS |
| | LDHLKPAMLELGKLSGHKHIFFDGEGTAGSFNQSVSAL |
| | RKKNVQAIGAIYHVFDFFLPEWRAQAKSKEYAKTGMK |
| | LKERLAMLVALFKNDRSEGYAQDIHLHPFYIIHSHEDFIE |
| | RFMKRLDDNQEGEMGKDPNSVYEFKRTRSWWKLKDE |
| | DSEDGEIIDFEPGDPDSGFANTLGKIVIRLENGVIVRASGI |
| | KHKYLDEIWNNKEKYRGRIVEVHCHEKTPDGSLRHPRL |
| | KWPRCLRDTEDRIGDKE (SEQ ID NO: 11) |
| | GENBANK NO: |
| | WP_002851175.1 |
| | MRFVFLICCACLVFANEILLLSKFDKQDFNSKDFNAYLM |
| | SEKLDGVRGIWDGKYLKTRQNYKIKTPDFFTKNFPPFAI |
| | DGELWIARNKFDEISALIRSGDSNLTLWKEVTYNIFDVP |
| | NACEEFQISTCTLKNRLAVLEEYLQKYPSAYIKIISQIPVE |
| | NQNNLNQFYESIIKNQGEGIVIRKNLSPYEKGRSKNATK |
| | LKPYDDAECELVGFRKGKGKFENQVGALLCKMPNGQII |
| | KIGSGLKDEDRKNPPKIGSIVTYKFNGLTKNSLPRFPVFL |
| | RIRDENP (SEQ ID NO: 12) |
| | GENBANK NO: |
| | YP_009842857.1 |
| | MKPMFASMADPGLLEGQLPIFMSPKIDGFRAFIHDGHAL |
| | TRSFKPQANAAINQYLSNPIFNGLDGELVCGDITDPKVF |
| | QKSSGDLRRRDGEPDWSFHVFDDFTDPRMPTDERLDAA |
| | EHRVKELRERFEMTRIHLVEQELVTSVEQMTETELRHV |
| | GLGFEGSIGKKKNGIYKFGRSTAKEGHCVKFKRYDSEE |
| | AEIIGVEELMINENEAFIDELGYTARSSHAENLVPSGMV |
| | GSFVCRNEKVWPGQTFRVSASSITHDEKQRLWKDRESL |
| | NGQVIRFTHFPHGAKDKPRHAVFDCWLDGWGASH |
| | (SEQ ID NO: 13) |

### REFERENCES

Each of the Following References are Hereby Incorporated in their Entireties
1. Crick, F. & Watson, J. D. The Molecular Structure of Nucleic Acids: The Classic Papers from Nature, 25 Apr. 1953. (1953).
2. Ellegren, H. & Galtier, N. Determinants of genetic diversity. Nature Reviews Genetics vol. 17 422-433 (2016).
3. Smith, M. J. et al. Loss-of-function mutations in SMARCE1 cause an inherited disorder of multiple spinal meningiomas. Nat. Genet. 45, 295-298 (2013).
4. Zahn, L. M. Mapping genotype to phenotype. Science vol. 362 555.4-556 (2018).
5. Ludwig, L. S. et al. Lineage Tracing in Humans Enabled by Mitochondrial Mutations and Single-Cell Genomics. Cell 176, 1325-1339.e22 (2019).
6. Salk, J. J. & Horwitz, M. S. Passenger mutations as a marker of clonal cell lineages in emerging neoplasia. Semin. Cancer Biol. 20, 294-303 (2010).
7. MacArthur, D. G. et al. Guidelines for investigating causality of sequence variants in human disease. Nature 508, 469-476 (2014).
8. Ashley, E. A. Towards precision medicine. Nat. Rev. Genet. 17, 507-522 (2016).
9. Gerlinger, M. et al. Intratumor heterogeneity and branched evolution revealed by multiregion sequencing. N. Engl. J. Med. 366, 883-892 (2012).
10. Vasan, N., Baselga, J. & Hyman, D. M. A view on drug resistance in cancer. Nature vol. 575 299-309 (2019).
11. Zahn, L. M. Somatic mosaicism in normal tissues. Science vol. 364 966.10-968 (2019).
12. Genovese, G. et al. Clonal hematopoiesis and blood-cancer risk inferred from blood DNA sequence. N. Engl. J. Med. 371, 2477-2487 (2014).
13. Anzalone, A. V., Koblan, L. W. & Liu, D. R. Genome editing with CRISPR-Cas nucleases, base editors, transposases and prime editors. Nature Biotechnology vol. 38 824-844 (2020).
14. Matsumura, S., Fujita, Y., Yamane, M., Morita, O. & Honda, H. A genome-wide mutation analysis method enabling high-throughput identification of chemical mutagen signatures. Sci. Rep. 8, 9583 (2018).
15. D'Gama, A. M. & Walsh, C. A. Somatic mosaicism and neurodevelopmental disease. Nat. Neurosci. 21, 1504-1514 (2018).
16. Bell, A. D. et al. Insights into variation in meiosis from 31,228 human sperm genomes. Nature 583, 259-264 (2020).
17. Blauwkamp, T. A. et al. Analytical and clinical validation of a microbial cell-free DNA sequencing test for infectious disease. Nat Microbiol 4, 663-674 (2019).
18. Lennon, A. M. et al. Feasibility of blood testing combined with PET-CT to screen for cancer and guide intervention. Science 369, (2020).
19. Lanman, R. B. et al. Analytical and Clinical Validation of a Digital Sequencing Panel for Quantitative, Highly Accurate Evaluation of Cell-Free Circulating Tumor DNA. PLoS One 10, e0140712 (2015).
20. Chen, L., Liu, P., Evans, T. C., Jr & Ettwiller, L. M. DNA damage is a pervasive cause of sequencing errors, directly confounding variant identification. Science 355, 752-756 (2017).
21. Costello, M. et al. Discovery and characterization of artifactual mutations in deep coverage targeted capture sequencing data due to oxidative DNA damage during sample preparation. Nucleic Acids Res. 41, e67 (2013).
22. Wong, S. Q. et al. Sequence artefacts in a prospective series of formalin-fixed tumours tested for mutations in hotspot regions by massively parallel sequencing. BMC Med. Genomics 7, 23 (2014).
23. Salk, J. J., Schmitt, M. W. & Loeb, L. A. Enhancing the accuracy of next-generation sequencing for detecting rare and subclonal mutations. Nat. Rev. Genet. 19, 269-285 (2018).
24. Schmitt, M. W. et al. Detection of ultra-rare mutations by next-generation sequencing. Proc. Natl. Acad. Sci. U.S.A. 109, 14508-14513 (2012).
25. Parsons, H. A. et al. Sensitive Detection of Minimal Residual Disease in Patients Treated for Early-Stage Breast Cancer. Clin. Cancer Res. 26, 2556-2564 (2020).
26. Zhang, A. et al. Solid-phase enzyme catalysis of DNA end repair and 3′ A-tailing reduces GC-bias in next-generation sequencing of human genomic DNA. Sci. Rep. 8, 15887 (2018).
27. Jiang, P. et al. Detection and characterization of jagged ends of double-stranded DNA in plasma. Genome Res. 30, 1144-1153 (2020).
28. Zatopek, K. M. et al. RADAR-seq: A RAre DAmage and Repair sequencing method for detecting DNA damage on a genome-wide scale. DNA Repair 80, 36-44 (2019).
29. Xiao, C.-L. et al. N6-Methyladenine DNA Modification in the Human Genome. Mol. Cell 71, 306-318.e7 (2018).
30. Lee, D.-H. Oxidative DNA damage induced by copper and hydrogen peroxide promotes CG->TT tandem mutations at methylated CpG dinucleotides in nucleotide excision repair-deficient cells. Nucleic Acids Research vol. 30 3566-3573 (2002).
31. Adalsteinsson, V. A. et al. Scalable whole-exome sequencing of cell-free DNA reveals high concordance with metastatic tumors. Nat. Commun. 8, 1324 (2017).

### Additional Embodiments

Embodiment 1. A method of preparing a nucleic acid sample (sample) for sequencing that minimizes propagation of false mutations due to amplification of nucleotide damage or base pair mismatches, wherein at least a portion of the sample is double-stranded, comprising adding a sample to a reaction vessel and: (a) contacting the sample to one or more enzymes capable of: (i) excising one or more damaged bases from the sample; (ii) cleaving one or more abasic sites, and processing the resulting ends to be compatible with extension by a DNA polymerase and/or ligation by a DNA ligase; (iii) digesting 5′ overhangs; (b) contacting the sample with one or more of: (i) a DNA-dependent DNA polymerase lacking both strand displacement and 5′ exonuclease activity but capable of fill-in of single-stranded segments of the sample and digesting 3′ overhangs of the sample; and (ii) an enzyme capable of phosphorylating the 5′ ends of the strands of the sample; and (c) contacting the sample with a DNA ligase capable of sealing nicks.

Embodiment 2. The method of embodiment 1, further comprising: (d) preparing the sample for adapter ligation, wherein the preparing comprises: (i) adding deoxyadenosine monophosphate (dAMP) to the 3′ ends of the strands of the sample (dA-tailing); or (ii) optionally blunting the ends of the sample.

Embodiment 3. The method of embodiment 2, wherein the dA-tailing comprises, contacting the sample with an enzyme capable of incorporating deoxyadenosine monophosphate (dAMP) to the 3′ ends of a strand of the sample and contacting the sample with dNTPs.

Embodiment 4. The method of embodiment 2 or embodiment 3, wherein enzymes and/or dNTPs used in steps (a)-(c) are substantially removed from the reaction vessel prior to dA-tailing.

Embodiment 5. The method of embodiment 2 or any one of embodiments 3-4, wherein the dNTPs contacted with the sample substantially comprise dATPs.

Embodiment 6. The method of embodiment 1 or any one of embodiments 2-5, where the sample is contacted by the one or more enzymes of step (a) and incubated for at least 5 minutes (min) prior to proceeding with any subsequent steps of the method.

Embodiment 7. The method of embodiment 1 or any one of embodiments 2-6, where the sample is contacted by the one or more enzymes of step (a) and incubated for at least 25 minutes (min) prior to proceeding with any subsequent steps of the method.

Embodiment 8. The method of embodiment 1 or any one of embodiments 2-7, where the sample is contacted by the one or more enzymes of step (a) and incubated for at least 30 minutes (min) prior to proceeding with any subsequent steps of the method.

Embodiment 9. The method of embodiment 1 or any one of embodiments 2-8, where the sample is contacted by the one or more enzymes of step (b) and incubated for at least 5 minutes (min) prior to proceeding with any subsequent steps of the method.

Embodiment 10. The method of embodiment 1 or any one of embodiments 2-9, where the sample is contacted by the one or more enzymes of step (b) and incubated for at least 25 minutes (min) prior to proceeding with any subsequent steps of the method.

Embodiment 11. The method of embodiment 1 or any one of embodiments 2-10, where the sample is contacted by the one or more enzymes of step (b) and incubated for at least 30 minutes (min) prior to proceeding with any subsequent steps of the method.

Embodiment 12. The method of embodiment 1 or any one of embodiments 2-11, where the sample is contacted by the one or more enzymes of step (c) and incubated for at least 15 minutes (min) prior to proceeding with any subsequent steps of the method.

Embodiment 13. The method of embodiment 1 or any one of embodiments 2-12, where the sample is contacted by the one or more enzymes of step (c) and incubated for at least 30 minutes (min) prior to proceeding with any subsequent steps of the method.

Embodiment 14. The method of embodiment 1 or any one of embodiments 2-13, where the sample is contacted by the one or more enzymes of step (c) and incubated for at least 45 minutes (min) prior to proceeding with any subsequent steps of the method.

Embodiment 15. The method of embodiment 2 or any one of embodiments 3-14, where the sample is contacted by the one or more enzymes of step (d) and incubated for at least 40 minutes (min) prior to proceeding with any subsequent steps of the method.

Embodiment 16. The method of embodiment 2 or any one of embodiments 3-15, where the sample is contacted by the one or more enzymes of step (d) and incubated for at least 60 minutes (min) prior to proceeding with any subsequent steps of the method.

Embodiment 17. The method of embodiment 2 or any one of embodiments 3-16, where the sample is contacted by the one or more enzymes of step (d) and incubated for at least 70 minutes (min) prior to proceeding with any subsequent steps of the method.

Embodiment 18. The method of embodiment 1 or any one of embodiments 2-17, wherein step (a) is carried out at a temperature between about 32° C. to about 42° C.

Embodiment 19. The method of embodiment 1 or any one of embodiments 2-18, wherein step (a) is carried out at a temperature between about 35° C. to about 39° C.

Embodiment 20. The method of embodiment 1 or any one of embodiments 2-19, wherein step (b) is carried out at a temperature between about 32° C. to about 42° C.

Embodiment 21. The method of embodiment 1 or any one of embodiments 2-20, wherein step (b) is carried out at a temperature between about 35° C. to about 39° C.

Embodiment 22. The method of embodiment 1 or any one of embodiments 2-21, wherein step (c) is carried out at a temperature between about 30° C. to about 70° C.

Embodiment 23. The method of embodiment 1 or any one of embodiments 2-22, wherein step (c) is carried out at a temperature between about 33° C. to about 67° C.

Embodiment 24. The method of embodiment 2 or any one of embodiments 3-23, wherein step (d) is carried out at a temperature between about 18° C. to about 69° C.

Embodiment 25. The method of embodiment 2 or any one of embodiments 3-24, wherein step (d) is carried out at a temperature between about 20° C. to about 67° C.

Embodiment 26. The method of embodiment 1 or any one of embodiments 2-25, wherein prior to step (a) the sample has been: (i) fragmented; or (ii) cleaved and tagged (tagmented).

Embodiment 27. The method of embodiment 27, wherein the fragmentation was by: (a) physical fragmentation; (b) enzymatic fragmentation; and/or (c) chemical fragmentation.

Embodiment 28. The method of embodiment 26 or embodiment 27, wherein the fragmentation was by physical fragmentation.

Embodiment 29. The method of embodiment 26 or embodiment 27, wherein the fragmentation was by enzymatic fragmentation.

Embodiment 30. The method of embodiment 26 or embodiment 27, wherein the fragmentation was by chemical fragmentation.

Embodiment 31. The method of embodiment 1 or any one of embodiments 2-30, wherein step (a) comprises contacting the sample with one or more enzymes selected from the group consisting of: (1) endonuclease IV (EndoIV); (2) formamidopyrimidine [fapy]-DNA glycosylase (Fpg); (3) uracil-DNA glycosylase (UDG); (4) T4 pyrimidine DNA glycosylase (T4 PDG); and (5) endonuclease VIII (EndoVIII). (6) exonuclease VII (ExoVII)

Embodiment 32. The method of embodiment 1 or any one of embodiments 2-31, wherein the simultaneous activity of the one or more enzymes catalyze the following DNA modifications on the sample: (1) excision of damaged bases; and (2) cleaving of abasic sites and processing the resulting ends to be compatible with extension by a DNA polymerase and/or ligation by a DNA ligase.

Embodiment 33. The method of embodiment 1 or any one of embodiments 2-32, wherein the damaged bases are selected from the group consisting of: uracil; 8′oxoG; an oxidized pyrimidine; and a cyclobutane pyrimidine dimer.

Embodiment 34. The method of embodiment 1 or any one of embodiments 2-33, wherein the 5′ overhang of at least one strand of the sample is at least 10 nucleobases in length.

Embodiment 35. The method of embodiment 1 or any one of embodiments 2-34, wherein the 5′ overhang of at least one strand of the sample is at least 75 nucleobases in length.

Embodiment 36. The method of embodiment 1 or any one of embodiments 2-35, wherein the 3′ overhang of at least one strand of the sample is at least 10 nucleobases in length.

Embodiment 37. The method of embodiment 1 or any one of embodiments 2-36, wherein the 3′ overhang of at least one strand of the sample is at least 75 nucleobases in length.

Embodiment 38. The method of embodiment 1 or any one of embodiments 2-37, wherein the one or more enzymes digests the 5′ overhang of at least one strand of the sample to less than 16 nucleobases in length.

Embodiment 39. The method of embodiment 1 or any one of embodiments 2-38, wherein the one or more enzymes digests the 5′ overhang of at least one strand of the sample to less than 8 nucleobases in length.

Embodiment 40. The method of embodiment 1 or any one of embodiments 2-39, wherein the one or more enzymes digests the 3′ overhang of at least one strand of the sample to less than 16 nucleobases in length.

Embodiment 41. The method of embodiment 1 or any one of embodiments 2-40, wherein the one or more enzymes digests the 3′ overhang of at least one strand of the sample to less than 8 nucleobases in length.

Embodiment 42. The method of embodiment 1 or any one of embodiments 2-41, wherein endonuclease IV (EndoIV) cleaves abasic sites.

Embodiment 43. The method of embodiment 1 or any one of embodiments 2-41, wherein formamidopyrimidine [fapy]-DNA glycosylase excises damaged purines.

Embodiment 44. The method of embodiment 1 or any one of embodiments 2-41, wherein uracil-DNA glycosylase (UDG) excises uracil.

Embodiment 45. The method of embodiment 1 or any one of embodiments 2-41, wherein T4 pyrimidine DNA glycosylase (T4 PDG) excises cyclobutane pyrimidine dimers.

Embodiment 46. The method of embodiment 1 or any one of embodiments 2-41, wherein endonuclease VIII (EndoVIII) excises damaged pyrimidines.

Embodiment 47. The method of embodiment 1 or any one of embodiments 2-46, wherein the DNA ligase is HiFi Taq DNA ligase.

Embodiment 48. The method of embodiment 1 or any one of embodiments 2-47, wherein the DNA ligase has nick sealing activity but lacks end-joining activity.

Embodiment 49. The method of embodiment 2 or any one of embodiments 3-48, wherein the step (b) comprises contacting the DNA fragment with a polynucleotide kinase (Pnk).

Embodiment 50. The method of embodiment 49, wherein the Pnk is a T4 polynucleotide kinase.

Embodiment 51. The method of embodiment 31 or any one of embodiments 32-50, wherein: (a) the endonuclease IV (EndoIV) comprises an amino acid sequence with at least 70% identity to an amino acid sequence of SEQ ID NO: 3; (b) the formamidopyrimidine [fapy]-DNA glycosylase (Fpg) comprises an amino acid sequence with at least 70% identity to an amino acid sequence of SEQ ID NO: 4; (c) the uracil-DNA glycosylase (UDG) comprises an amino acid sequence with at least 70% identity to an amino acid sequence selected from the group consisting of: SEQ ID NO: 5-7; (d) the T4 pyrimidine DNA glycosylase (T4 PDG) comprises an amino acid sequence with at least 70% identity to any known sequence; (e) the endonuclease VIII (EndoVIII) comprises an amino acid sequence with at least 70% identity to an amino acid sequence selected from the group consisting of: SEQ ID NO: 8-9; and/or (f) the exonuclease VII (ExoVII) comprises an amino acid sequence with at least 70% identity to any known sequence.

Embodiment 52. The method of embodiment 49 or any one of embodiments 50-51, wherein the polynucleotide kinase comprises an amino acid sequence with at least 70% identity to an amino acid sequence of SEQ ID NO: 8.

Embodiment 53. The method of embodiment 1 or any one of embodiments 2-52, wherein: (1) the DNA-dependent DNA polymerase comprises an amino acid sequence with at least 70% identity to any known sequence; and/or (2) the DNA ligase comprises an amino acid sequence with at least 70% identity to an amino acid sequence selected from the group consisting of: SEQ ID NO: 11-13.

Embodiment 54. A method of duplex sequencing that mitigates false mutation detection, comprising: (A1) obtaining a nucleic acid to be sequenced; (A2) performing the method of embodiment 1 or any one of embodiments 2-52; (A3) duplex sequencing the sample; and (A4) identifying mutations by computational analysis.

Embodiment 55. A method of reducing artifact in duplex sequencing, comprising: (A1) obtaining a nucleic acid to be sequenced; (A2) performing the method of embodiment 1 or any one of embodiments 2-52; and (A3) duplex sequencing the sample.

Embodiment 56. A method of reducing synthetic strand synthesis during nucleic acid sample preparation for sequencing, comprising: (A1) obtaining a nucleic acid to be sequenced; and (A2) performing the method of embodiment 1 or any one of embodiments 2-52.

Embodiment 57. A method of increasing the accuracy of mutation identification, comprising: (A1) obtaining a nucleic acid to be sequenced; (A2) performing the method of embodiment 1 or any one of embodiments 2-52; (A3) duplex sequencing the sample; and (A4) identifying mutations by computational analysis.

Embodiment 58. A kit comprising: (a) reagents to perform the methods any of embodiments 1-57; and (b) a container.

Embodiment 59. The kit of embodiment 58, further comprising a reaction vessel.

Embodiment 60. The kit of any one of embodiments 58 or embodiment 59, wherein the reagents comprise: (a) one or more of: endonuclease IV (EndoIV); formamidopyrimidine [fapy]-DNA glycosylase (Fpg); uracil-DNA glycosylase (UDG); T4 pyrimidine DNA glycosylase (T4 PDG); and/or endonuclease VIII (EndoVIII); exonuclease VII (ExoVII), T4 polynuclease kinase (T4 Pnk), T4 DNA polymerase, HiFi Taq ligase, Klenow fragment, and Taq polymerase and/or (b) dNTPs.

Embodiment 61. The kit of embodiment 58 or any one of embodiments 59-60, wherein the kit further comprises reagents and materials to fragment the sample.

Embodiment 62. A method of preparing a nucleic acid sample (sample) wherein at least a portion of the sample is double-stranded, comprising adding a sample to a reaction vessel and: (a) contacting the sample with one or more enzymes capable of: (i) phosphorylating the 5′ ends of the strands of the sample; adding a 3′ hydroxyl moiety to the 3′ ends of the strands of the sample; and (ii) sealing nicks; (b) contacting the sample with one or more of an enzyme capable of removing the 5′ and 3′ overhangs while also digesting gap regions to produce blunted duplexes; and (c) adding deoxyadenosine monophosphate (dAMP) to the 3′ ends of the strands of the sample (dA-tailing).

Embodiment 63. The method of embodiment 62, wherein the enzyme used in step (a)(1) comprises: T4 polynucleotide kinase, HiFi Taq Ligase, or a combination thereof.

Embodiment 64. The method of embodiment 62 or embodiment 63, wherein the enzyme used in step (b) is Nuclease S1.

In addition to the embodiments expressly described herein, it is to be understood that all of the features disclosed in this disclosure may be combined in any combination (e.g., permutation, combination). Each element disclosed in the disclosure may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, and can make various changes and modifications of the invention to adapt it to various usages and conditions. Thus, other embodiments are also within the claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following embodiments.

What is claimed is:

1. A method of preparing a nucleic acid sample (sample) wherein at least a portion of the sample is double-stranded, comprising adding a sample to a reaction vessel and:
(a) contacting the sample to one or more enzymes capable of:
(i) excising one or more damaged bases from the sample;
(ii) cleaving one or more abasic sites, and processing the resulting ends to be compatible with extension by a DNA polymerase and/or ligation by a DNA ligase;
(iii) digesting 5′ overhangs;
(b) contacting the sample with one or more of:
(i) a DNA-dependent DNA polymerase lacking both strand displacement and 5′ exonuclease activity but capable of fill-in of single-stranded segments of the sample and digesting 3′ overhangs of the sample; and
(ii) an enzyme capable of phosphorylating the 5′ ends of the strands of the sample; and
(c) contacting the sample with a DNA ligase capable of sealing nicks.

2. The method of claim 1, further comprising:
(d) preparing the sample for adapter ligation, wherein the preparing comprises:
(i) adding deoxyadenosine monophosphate (dAMP) to the 3′ ends of the strands of the sample (dA-tailing); or
(ii) optionally further blunting the ends of the sample.

3. The method of claim 2, wherein the dA-tailing comprises, contacting the sample with an enzyme capable of incorporating deoxyadenosine monophosphate (dAMP) to the 3′ ends of a strand of the sample and contacting the sample with dNTPs.

4. The method of claim 2 or claim 3, wherein the enzymes and/or dNTPs used in steps (a)-(c) are substantially removed from the reaction vessel prior to dA-tailing.

5. The method of claim 2 or any one of claims 3-4, wherein the dNTPs contacted with the sample substantially comprise dATPs.

6. The method of claim 1 or any one of claims 2-5, wherein the sample is contacted by the one or more enzymes of step (a) and incubated for at least 5 minutes (min) prior to proceeding with any subsequent steps of the method.

7. The method of claim 1 or any one of claims 2-6, wherein the sample is contacted by the one or more enzymes of step (a) and incubated for at least 25 minutes (min) prior to proceeding with any subsequent steps of the method.

8. The method of claim 1 or any one of claims 2-7, wherein the sample is contacted by the one or more enzymes of step (a) and incubated for at least 30 minutes (min) prior to proceeding with any subsequent steps of the method.

9. The method of claim 1 or any one of claims 2-8, wherein the sample is contacted by the one or more enzymes of step (b) and incubated for at least 5 minutes (min) prior to proceeding with any subsequent steps of the method.

10. The method of claim 1 or any one of claims 2-9, wherein the sample is contacted by the one or more enzymes of step (b) and incubated for at least 25 minutes (min) prior to proceeding with any subsequent steps of the method.

11. The method of claim 1 or any one of claims 2-10, wherein the sample is contacted by the one or more enzymes of step (b) and incubated for at least 30 minutes (min) prior to proceeding with any subsequent steps of the method.

12. The method of claim 1 or any one of claims 2-11, wherein the sample is contacted by the one or more enzymes of step (c) and incubated for at least 15 minutes (min) prior to proceeding with any subsequent steps of the method.

13. The method of claim 1 or any one of claims 2-12, wherein the sample is contacted by the one or more enzymes of step (c) and incubated for at least 30 minutes (min) prior to proceeding with any subsequent steps of the method.

14. The method of claim 1 or any one of claims 2-13, wherein the sample is contacted by the one or more enzymes of step (c) and incubated for at least 45 minutes (min) prior to proceeding with any subsequent steps of the method.

15. The method of claim 2 or any one of claims 3-14, wherein the sample is contacted by the one or more enzymes of step (d) and incubated for at least 40 minutes (min) prior to proceeding with any subsequent steps of the method.

16. The method of claim 2 or any one of claims 3-15, wherein the sample is contacted by the one or more enzymes of step (d) and incubated for at least 60 minutes (min) prior to proceeding with any subsequent steps of the method.

17. The method of claim 2 or any one of claims 3-16, wherein the sample is contacted by the one or more enzymes of step (d) and incubated for at least 70 minutes (min) prior to proceeding with any subsequent steps of the method.

18. The method of claim 1 or any one of claims 2-17, wherein step (a) is carried out at a temperature between about 32° C. to about 42° C.

19. The method of claim 1 or any one of claims 2-18, wherein step (a) is carried out at a temperature between about 35° C. to about 39° C.

20. The method of claim 1 or any one of claims 2-19, wherein step (b) is carried out at a temperature between about 32° C. to about 42° C.

21. The method of claim 1 or any one of claims 2-20, wherein step (b) is carried out at a temperature between about 35° C. to about 39° C.

22. The method of claim 1 or any one of claims 2-21, wherein step (c) is carried out at a temperature between about 30° C. to about 70° C.

23. The method of claim 1 or any one of claims 2-22, wherein step (c) is carried out at a temperature between about 33° C. to about 67° C.

24. The method of claim 2 or any one of claims 3-23, wherein step (d) is carried out at a temperature between about 18° C. to about 69° C.

25. The method of claim 2 or any one of claims 3-24, wherein step (d) is carried out at a temperature between about 20° C. to about 67° C.

26. The method of claim 1 or any one of claims 2-25, wherein prior to step (a) the sample has been:
(i) fragmented; or
(ii) cleaved and tagged (tagmented).

27. The method of claim 27, wherein the fragmentation was by:
(a) physical fragmentation;
(b) enzymatic fragmentation; and/or
(c) chemical fragmentation.

28. The method of claim 26 or claim 27, wherein the fragmentation was by physical fragmentation.

29. The method of claim 26 or claim 27, wherein the fragmentation was by enzymatic fragmentation.

30. The method of claim 26 or claim 27, wherein the fragmentation was by chemical fragmentation.

31. The method of claim 1 or any one of claims 2-30, wherein step (a) comprises contacting the sample with one or more enzymes selected from the group consisting of:
(1) endonuclease IV (EndoIV);
(2) formamidopyrimidine [fapy]-DNA glycosylase (Fpg);
(3) uracil-DNA glycosylase (UDG);
(4) T4 pyrimidine DNA glycosylase (T4 PDG); and
(5) endonuclease VIII (EndoVIII).
(6) exonuclease VII (ExoVII)

32. The method of claim 1 or any one of claims 2-31, wherein the simultaneous activity of the one or more enzymes catalyze the following DNA modifications on the sample:
(1) excision of damaged bases; and
(2) cleaving of abasic sites and processing the resulting ends to be compatible with extension by a DNA polymerase and/or ligation by a DNA ligase.

33. The method of claim 1 or any one of claims 2-32, wherein the damaged bases are selected from the group consisting of: uracil; 8′oxoG; an oxidized pyrimidine; and a cyclobutane pyrimidine dimer.

34. The method of claim 1 or any one of claims 2-33, wherein the 5′ overhang of at least one strand of the sample is at least 10 nucleobases in length.

35. The method of claim 1 or any one of claims 2-34, wherein the 5′ overhang of at least one strand of the sample is at least 75 nucleobases in length.

36. The method of claim 1 or any one of claims 2-35, wherein the 3′ overhang of at least one strand of the sample is at least 10 nucleobases in length.

37. The method of claim 1 or any one of claims 2-36, wherein the 3′ overhang of at least one strand of the sample is at least 75 nucleobases in length.

38. The method of claim 1 or any one of claims 2-37, wherein the one or more enzymes digests the 5′ overhang of at least one strand of the sample to less than 16 nucleobases in length.

39. The method of claim 1 or any one of claims 2-38, wherein the one or more enzymes digests the 5′ overhang of at least one strand of the sample to less than 8 nucleobases in length.

40. The method of claim 1 or any one of claims 2-39, wherein the one or more enzymes digests the 3′ overhang of at least one strand of the sample to less than 16 nucleobases in length.

41. The method of claim 1 or any one of claims 2-40, wherein the one or more enzymes digests the 3′ overhang of at least one strand of the sample to less than 8 nucleobases in length.

42. The method of claim 1 or any one of claims 2-41, wherein endonuclease IV (EndoIV) cleaves abasic sites.

43. The method of claim 1 or any one of claims 2-41, wherein formamidopyrimidine [fapy]-DNA glycosylase excises damaged purines.

44. The method of claim 1 or any one of claims 2-41, wherein uracil-DNA glycosylase (UDG) excises uracil.

45. The method of claim 1 or any one of claims 2-41, wherein T4 pyrimidine DNA glycosylase (T4 PDG) excises cyclobutane pyrimidine dimers.

46. The method of claim 1 or any one of claims 2-41, wherein endonuclease VIII (EndoVIII) excises damaged pyrimidines.

47. The method of claim 1 or any one of claims 2-46, wherein the DNA ligase is HiFi Taq DNA ligase.

48. The method of claim 1 or any one of claims 2-47, wherein the DNA ligase has nick sealing activity but lacks end-joining activity.

49. The method of claim 2 or any one of claims 3-48, wherein the step (b) comprises contacting the DNA fragment with a polynucleotide kinase (Pnk).

50. The method of claim 49, wherein the Pnk is a T4 polynucleotide kinase.

51. The method of claim 31 or any one of claims 32-50, wherein:
(a) the endonuclease IV (EndoIV) comprises an amino acid sequence with at least 70% identity to an amino acid sequence of SEQ ID NO: 3;
(b) the formamidopyrimidine [fapy]-DNA glycosylase (Fpg) comprises an amino acid sequence with at least 70% identity to an amino acid sequence of SEQ ID NO: 4;
(c) the uracil-DNA glycosylase (UDG) comprises an amino acid sequence with at least 70% identity to an amino acid sequence selected from the group consisting of: SEQ ID NO: 5-7;
(d) the T4 pyrimidine DNA glycosylase (T4 PDG) comprises an amino acid sequence with at least 70% identity to any known sequence;
(e) the endonuclease VIII (EndoVIII) comprises an amino acid sequence with at least 70% identity to an amino acid sequence selected from the group consisting of: SEQ ID NO: 6-7; and/or
(f) the exonuclease VII (ExoVII) comprises an amino acid sequence with at least 70% identity to any known amino acid sequence.

52. The method of claim 49 or any one of claims 50-51, wherein the polynucleotide kinase comprises an amino acid sequence with at least 70% identity to an amino acid sequence of SEQ ID NO: 10.

53. The method of claim 1 or any one of claims 2-52, wherein:
(1) the DNA-dependent DNA polymerase comprises an amino acid sequence with at least 70% identity to any known or available amino acid sequence; and/or
(2) the DNA ligase comprises an amino acid sequence with at least 70% identity to an amino acid sequence selected from the group consisting of: SEQ ID NO: 11-13.

54. A method of sequencing that mitigates false mutation detection, comprising:
(A1) obtaining a nucleic acid to be sequenced;
(A2) performing the method of claim 1 or any one of claims 2-52;
(A3) sequencing the sample; and
(A4) identifying mutations by computational analysis.

55. A method of reducing artifact in duplex sequencing, comprising:
(A1) obtaining a nucleic acid to be sequenced;
(A2) performing the method of claim 1 or any one of claims 2-52; and
(A3) duplex sequencing the sample.

56. A method of reducing synthetic strand synthesis during nucleic acid sample preparation for sequencing, comprising:
(A1) obtaining a nucleic acid to be sequenced; and
(A2) performing the method of claim 1 or any one of claims 2-52.

57. A method of increasing the accuracy of mutation identification, comprising:
(A1) obtaining a nucleic acid to be sequenced;
(A2) performing the method of claim 1 or any one of claims 2-52;
(A3) duplex sequencing the sample; and
(A4) identifying mutations by computational analysis.

58. A kit comprising:
(a) reagents to perform the methods any of claims 1-57; and
(b) a container.

59. The kit of claim 58, further comprising a reaction vessel.

60. The kit of any one of claim 58 or claim 59, wherein the reagents comprise:
(a) one or more of: endonuclease IV (EndoIV); formamidopyrimidine [fapy]-DNA glycosylase (Fpg); uracil-DNA glycosylase (UDG); T4 pyrimidine DNA glycosylase (T4 PDG); and/or endonuclease VIII (EndoVIII); exonuclease VII (ExoVII), T4 polynuclease kinase (T4 Pnk), T4 DNA polymerase, HiFi Taq ligase, Klenow fragment, and Taq polymerase and/or
(b) dNTPs.

61. The kit of claim 58 or any one of claims 59-60, wherein the kit further comprises reagents and materials to fragment the sample.

62. A method of preparing a nucleic acid sample (sample) wherein at least a portion of the sample is double-stranded, comprising adding a sample to a reaction vessel and:
(a) contacting the sample with one or more enzymes capable of:
(i) phosphorylating the 5′ ends of the strands of the sample; adding a 3′ hydroxyl moiety to the 3′ ends of the strands of the sample; and
(ii) sealing nicks;
(b) contacting the sample with one or more of an enzyme capable of removing the 5′ and 3′ overhangs while also digesting gap regions to produce blunted duplexes; and
(c) adding deoxyadenosine monophosphate (dAMP) to the 3′ ends of the strands of the sample (dA-tailing).

63. The method of claim 62, wherein the enzyme used in step (a)(1) comprises: T4 polynucleotide kinase, HiFi Taq Ligase, or a combination thereof.

64. The method of claim 62 or claim 63, wherein the enzyme used in step (b) is Nuclease S1.
